SEQUENCE LISTING (31 sequences)

SEQ ID NO: 1 - protein, 434 aa, Homo sapiens
  cargo selection protein (mannose 6 phosphate receptor binding protein)
MSADGAEADGSTQVTVEEPVQQPSVVDRVASMPLISSTCDMVSAAYASTKESYPHIKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQVLSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 2 - protein, 434 aa, Homo sapiens
  cargo selection protein (mannose 6 phosphate receptor binding protein)
MSADGAEADGSTQVTVEEPVQQPSVVDRVASMPLISSTCDMVSAAYASTKESYPHIKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 3 - protein, 434 aa, Homo sapiens [sequence omitted]

SEQ ID NO: 4 - protein, 251 aa, Homo sapiens
  placental protein 17a2; PP17a2
MVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 5 - protein, 434 aa, Homo sapiens; identical to SEQ ID NO: 2
  cargo selection protein (mannose 6 phosphat receptor binding protein)

SEQ ID NO: 6 - protein, 434 aa, Homo sapiens; identical to SEQ ID NO: 2
  cargo selection protein (mannose 6 phosphate receptor binding protein)

SEQ ID NO: 7 - protein, 434 aa, Homo sapiens
  cargo selection protein TIP47
MSADGAEADGSTQVTVEEPVQQPSVVDRVASMPLISSTCDMVSAAYASTKESYPHVKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 8 - protein, 434 aa, Homo sapiens; identical to SEQ ID NO: 7
  Cargo selection protein (mannose 6 phosphate receptor binding protein)

SEQ ID NO: 9 - protein, 434 aa, Homo sapiens; identical to SEQ ID NO: 7
  cargo selection protein (mannose 6 phosphate receptor binding protein)

SEQ ID NO: 10 - protein, 251 aa, Homo sapiens; identical to SEQ ID NO: 4
  placental protein 17a1; PP17a1

SEQ ID NO: 11 - protein, 434 aa, Homo sapiens; identical to SEQ ID NO: 7
  Cargo selection protein TIP47 (47 kDa mannose 6-phosphate receptor-binding protein) (47 kDa MPR-binding protein) (Placental protein 17)

SEQ ID NO: 12 - protein, 434 aa, Homo sapiens; identical to SEQ ID NO: 7
  Sequence 1 from patent US 5989820

SEQ ID NO: 13 - DNA, 1305 bp, Homo sapiens
  Cargo selection protein (mannose 6 phosphate receptor binding protein) (TIP47), mRNA
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacatcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcggtcatgggctcccgcttgggccagatggtgttgagtggggtcgacacggtgctggggaagtcggaggagtgggcggacaaccacctgccccttacggatgccgaactggcccgcatcgccacatccctggatggctttgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggtcctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtag

SEQ ID NO: 14 - DNA, 1305 bp, Homo sapiens
  Cargo selection protein (mannose 6 phosphate receptor binding protein) (TIP47), mRNA
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacatcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcagtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtag

SEQ ID NO: 15 - DNA, 1305 bp, Homo sapiens
  Placental protein 17b1 (PP17) mRNA, complete cds
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacgtcaagactgtctgcgacgcagcagagaagggagtgaggaccctcacggcggctgctgtcagctgggctcagccgatcctctccaagctggagccccagattgcatcagccagcgaatacgcccacagggggctggacaagttggaggagaacctccccatgctgcggcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcggtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtag

SEQ ID NO: 16 - DNA, 756 bp, Homo sapiens
  Placental protein 17a2 (PP17) mRNA, complete cds
atggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtag

SEQ ID NO: 17 - DNA, 1305 bp, Homo sapiens; identical to SEQ ID NO: 14
  Cargo selection protein (mannose 6 phosphate receptor binding protein), clone MGC11117 IMAGE3833411, mRNA, complete cds

SEQ ID NO: 18 - DNA, 1305 bp, Homo sapiens; identical to SEQ ID NO: 14
  Cargo selection protein (mannose 6 phosphate receptor binding protein), clone MGC3816 IMAGE2905275, mRNA, complete cds

SEQ ID NO: 19 - DNA, 1305 bp, Homo sapiens
  Cargo selection protein TIP47 (TIP47) mRNA, complete cds
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacgtcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcagtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaaatag

SEQ ID NO: 20 - DNA, 1305 bp, Homo sapiens
  Cargo selection protein (mannose 6 phosphate receptor binding protein), clone MGC15516 IMAGE3028104, mRNA, complete cds
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacgtcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcagtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtag

SEQ ID NO: 21 - DNA, 1305 bp, Homo sapiens; identical to SEQ ID NO: 20
  Cargo selection protein (mannose 6 phosphate receptor binding protein), clone MGC2012 IMAGE2987965, mRNA, complete cds

SEQ ID NO: 22 - DNA, 756 bp, Homo sapiens; identical to SEQ ID NO: 16
  Placental protein 17a1 (PP17) mRNA, complete cds

SEQ ID NO: 23 - protein, 4 aa, Artificial
  Synthetic Peptide
DEVD

SEQ ID NO: 24 - protein, 7 aa, Artificial
  Synthetic Peptide
DEVDAPK

SEQ ID NO: 25 - protein, 8 aa, Artificial
  Synthetic Peptide
VDQMDGWK

SEQ ID NO: 26 - protein, 7 aa, Artificial
  Synthetic Peptide
DEVDARK

SEQ ID NO: 27 - protein, 5 aa, Artificial
  Synthetic Peptide
VDVAD

SEQ ID NO: 28 - protein, 8 aa, Artificial
  Synthetic Peptide
VDVADGWK

SEQ ID NO: 29 - protein, 8 aa, Artificial
  Synthetic Peptide
VDQVDGWK

SEQ ID NO: 30 - protein, 4 aa, Artificial
  Synthetic Peptide
VEID

SEQ ID NO: 31 - protein, 7 aa, Artificial
  Synthetic Peptide
VQVDGWK

(19) United States
(12) Patent Application Publication
(10) Pub. No.: US 2005/0004005 A1
(43) Pub. Date: Jan. 6, 2005

(54) Methods of treating diseases responsive to Induction of Apoptosis

(76) Inventors: Shailaja Kasibhatla, San Diego, CA (US); Sui Xiong Cai, San Diego, CA (US); Ben Tseng, San Diego, CA (US); Katayoun Alavi Jessen, San Diego, CA (US); Serguei Maliartchouk, San Diego, CA (US); Nicole Marion English, San Diego, CA (US); Jared Kuemmerle, Del Mar, CA (US); William E. Kemnitzer, Irvine, CA (US); Han-Zhong Zhang, San Diego, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/826,923

(22) Filed: Apr. 19, 2004

Related U.S. Application Data
(60) Provisional application No. 60/463,687, filed on Apr. 18, 2003.

(51) Int. Cl. A61K 38/00; A61K 31/47

(52) U.S. Cl. 514/2; 514/310; 514/364

(57) ABSTRACT

The present invention pertains to a method of treating, preventing or ameliorating a disease responsive to induction of the caspase cascade in an animal, comprising administering to the animal a compound which binds specifically to a Tail Interacting Protein Related Apoptosis Inducing Protein (TIPRAIP). The present invention also relates to screening methods useful for drug discovery of apoptosis inducing compounds. In particular, the screening methodology relates to using TIPRAIP as a target for the discovery of apoptosis activators useful as anticancer agents. The screening methods of the present invention can employ homogenous or heterogenous binding assays using purified or partially purified TIPRAIP; or whole cell assays using cells with altered levels of TIPRAIP. The invention also contemplates use of 3-(4-azidophenyl)-5-(3-chloro-thiophen-2-yl)-[1,2,4]-oxadiazole or a substituted 3-aryl-5-aryl-[1,2,4]-oxadiazole which bind TIPRAIP and can accordingly be used to raise antibodies useful for drug discovery. Alternatively, labeled 3-(4-azidophenyl)-5-(3-chloro-thiophen-2-yl)-[1,2,4]-oxadiazole (or a labeled substituted 3-aryl-5-aryl-[1,2,4]-oxadiazole) is used for competitive binding assays for drug discovery. Such assays afford high throughput screening of chemical libraries for apoptosis activators.

### CROSS REFERENCE TO RELATED APPLICATIONS

This application claims the benefit under 35 U.S.C. §119(e) of U.S. Provisional Application No. 60/463,687, filed Apr. 18, 2003, which is hereby wholly incorporated by reference.

### BACKGROUND OF THE INVENTION

1. Field of the Invention

The present invention relates to a method of treating, preventing or ameliorating a disease responsive to induction of the caspase cascade in an animal, comprising administering to the animal a compound which binds specifically to a Tail Interacting Protein Related Apoptosis Inducing Protein (TIPRAIP). The present invention also relates to methods for identifying such TIPRAIP binding compounds. The invention also relates to the use of biochemical and cell based screening assays to identify TIPRAIP binding compounds that may be administered to animals for treating, preventing or ameliorating a disease responsive to induction of the caspase cascade.

2. Related Art

Organisms eliminate unwanted cells by a process variously known as regulated cell death, programmed cell death or apoptosis. Such cell death occurs as a normal aspect of animal development, as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall (1981), pp. 9-34). A cell activates its internally encoded suicide program as a result of either internal or external signals. The suicide program is executed through the activation of a carefully regulated genetic program (Wyllie, et al., Int. Rev. Cyt. 68:251 (1980); Ellis, et al., Ann. Rev. Cell Bio. 7:663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

It has been found that a group of proteases are a key element in apoptosis (see, e.g., Thornberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, 2 of which are the pro-apoptotic (death-promoting) ced (for cell death abnormal) genes, ced-3 and ced-4. CED-3 is homologous to interleukin 1 beta-converting enzyme, a cysteine protease, which is now called caspase-1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activates the caspase cascade. This makes the cancer cells lose their capacity to undergo cellular suicide and the cells become cancerous. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (see, Schmitt, et al., Biochem. Cell. Biol. 75:301-314 (1997)). BCL-like proteins include BCL-xL and BAX-alpha, which appear to function upstream of caspase activation. BCL-xL appears to prevent activation of the apoptotic protease cascade, whereas BAX-alpha accelerates activation of the apoptotic protease cascade.

It has been shown that chemotherapeutic (anti-cancer) drugs can trigger cancer cells to undergo suicide by activating the dormant caspase cascade. This may be a crucial aspect of the mode of action of most, if not all, known anticancer drugs (Los, et al., Blood 90:3118-3129 (1997); Friesen, et al., Nat. Med. 2:574 (1996)). The mechanism of action of current cycle. In brief, the cell cycle refers to the stages through which cells normally progress during their lifetime. Normally, cells exist in a resting phase termed Gₒ. During multiplication, cells progress to a stage in which DNA synthesis occurs, termed S. Later, cell division, or mitosis occurs, in a phase called M. Antineoplastic drugs, such as cytosine arabinoside, hydroxyurea, 6-mercaptopurine, and methotrexate are S phase specific, whereas antineoplastic drugs, such as vincristine, vinblastine, and paclitaxel are M phase specific. Many slow growing tumors, e.g. colon cancers, exist primarily in the Gₒ phase, whereas rapidly proliferating normal tissues, for example bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, et al., eds., Goodman and Gilman's The Pharmacological Basis of Therapeutics, Ninth Edition, McGraw-Hill, New York (1996), pp. 1225-1287). Thus, it is clear that the possibility exists for the activation of the caspase cascade, although the exact mechanisms have heretofore not been clear. It is equally clear that insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could also involve the enhancement of the apoptotic process through the administration of appropriate caspase cascade activators and inducers of apoptosis.

### SUMMARY OF THE INVENTION

As described in nonprovisional U.S. patent application Ser. No. 10/164,705, filed Jun. 10, 2002 (Cai et al.); and in provisional U.S. Patent Application No. 60/433,953, filed Dec. 18, 2002 (Cai et al.), 3-(4-azidophenyl)-5-(3-chloro-thiophen-2-yl)-[1,2,4]-oxadiazole and substituted 3-aryl-5-aryl-[1,2,4]-oxadiazoles are potent and highly efficacious activators of the caspase cascade and activators of apoptosis. The present invention relates to the discovery that apoptosis is induced upon the binding of 3-(4-azidophenyl)-5-(3-chloro-thiophen-2-yl)-[1,2,4]-oxadiazole to a Tail Interacting Protein Related Apoptosis Inducing Protein (TIPRAIP). Such binding is a starting point for initiating the caspase cascade and apoptosis. The binding of 3-(4-azidophenyl)-5-(3-chloro-thiophen-2-yl)-[1,2,4]-oxadiazole or substituted 3-aryl-5-aryl-[1,2,4]-oxadiazoles to TIPRAIP results in induction of apoptosis in cells, typically within 24 to 48 hours.

Generally, the present invention relates to compounds which bind specifically to TIPRAIP and induce activation of the caspase cascade and apoptosis; pharmaceutical formulations of these compounds; methods of treating, preventing or ameliorating a disease responsive to induction of the caspase cascade in an animal, comprising administering to the animal such compounds; methods for identifying such TIPRAIP binding compounds; and use of homogenous, heterogenous, protein and/or cell based screening assays to identify TIPRAIP binding compounds that may be administered to animals for treating, preventing or ameliorating a disease responsive to induction of the caspase cascade.

A first embodiment of the invention relates to a method of treating, preventing or ameliorating a disease responsive to induction of the caspase cascade in an animal, comprising administering to the animal a compound which binds specifically to a TIPRAIP, wherein the compound induces activation of the caspase cascade in the animal and the disease is treated, prevented or ameliorated; with the proviso that the compound is not 3-(4-azidophenyl)-5-(3-chloro-thiophen-2-yl)-[1,2,4]-oxadiazole or a substituted 3-aryl-5-aryl-[1,2,4]-oxadiazole.

In this embodiment, the disease may be a hyperproliferative disease. The hyperproliferative disease may be a cancer. The cancer may be Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, or prostatic carcinomas. Alternatively, the disease may be an inflammatory disease. The compound may be identified by determining whether the compound binds specifically to TIPRAIP. The TIPRAIP may be a tail interacting protein.

The invention also relates to the discovery that TIPRAIPs are useful for screening for other apoptotic inducing agents. Such screening can employ TIPRAIPs, nucleotides which encode TIPRAIPs, nucleotides which hybridize to the nucleotides which encode TIPRAIPs, and combinations thereof.

In another embodiment, the invention pertains to a method of identifying potentially therapeutic anticancer compounds comprising: (a) contacting a TIPRAIP with one or more test compounds; and (b) monitoring whether the one or more test compounds binds to the TIPRAIP; wherein compounds which bind the TIPRAIP are potentially therapeutic anticancer compounds. The TIPRAIP may be a tail interacting protein.

The invention also pertains to the use of partially or fully purified TIPRAIPs which may be used in homogenous or heterogenous binding assays to screen a large number or library of compounds and compositions for their potential ability to induce apoptosis. Those compositions capable of binding to TIPRAIPs are potentially useful for inducing apoptosis in vivo. TIPRAIPs can be synthesized or isolated from cells which over express these polypeptides. Accordingly, the invention also relates to nucleotides that encode for TIPRAIPs; vectors comprising these nucleotides; and cells comprising these vectors.

In another embodiment of the invention, determining whether the compound binds specifically to TIPRAIP may comprise a competitive or noncompetitive homogeneous assay. The homogeneous assay may be a fluorescence polarization assay or a radioassay. Alternatively, determining whether the compound binds specifically to TIPRAIP may comprise a competitive heterogeneous assay. The heterogeneous assay may be a fluorescence assay, a radioassay or an assay comprising avidin and biotin. The TIPRAIP may comprise a detectable label. The label on the TIPRAIP may be selected from the group consisting of a fluorescent label and a radiolabel. Alternatively, 3-(4-azidophenyl)-5-(3-chloro-thiophen-2-yl)-[1,2,4]-oxadiazole or a substituted 3-aryl-5-aryl-[1,2,4]-oxadiazole may comprise a detectable label. The label on 3-(4-azidophenyl)-5-(3-chloro-thiophen-2-yl)-[1,2,4]-oxadiazole or the substituted 3-aryl-5-aryl-[1,2,4]-oxadiazole may be selected from the group consisting of a fluorescent label and a radiolabel.

The invention also pertains to cells with altered levels of expression of TIPRAIPs which may be used in cell-based screening assays to screen a large number or library of compounds and compositions for their ability to induce apoptosis. Such screening assays may be performed with intact cells and afford the identification of potentially therapeutic antineoplastic compositions. In one embodiment, cells have altered levels of expression of TIPRAIPs by use of antisense nucleotides or RNA interference. In another embodiment, cells have reduced levels of expression of TIPRAIPs by modifying or knocking out the genes in cellular genomic or mitochondrial DNA encoding TIPRAIPs. In another embodiment, vectors are introduced into the cells thereby elevating levels of expression of TIPRAIPs. In another embodiment, cellular genomic or mitochondrial DNA is modified thereby elevating levels of expression of TIPRAIPs. In a further embodiment, an TIPRAIP binding compound is determined in cell-based screening by i) introducing a compound to a cell having an altered level of expression of TIPRAIPs; and ii) monitoring the extent to which the compound induces apoptosis by measuring observable changes in reporter compounds' response to the caspase cascade. Hence, in another embodiment of the invention, the TIPRAIP may be present in cells in vitro.

The invention also relates to the use of 3-(4-azidophenyl)-5-(3-chloro-thiophen-2-yl)-[1,2,4]-oxadiazole or a substituted 3-aryl-5-aryl-[1,2,4]-oxadiazole for raising antibodies which can be used to screen chemical libraries for other compositions that bind TIPRAIPs, or that activate apoptosis. Accordingly, in another embodiment, the invention pertains to a method of identifying potentially therapeutic anticancer compounds comprising: (a) contacting an antibody to 3-(4-azidophenyl)-5-(3-chloro-thiophen-2-yl)-[1,2,4]-oxadiazole or a substituted 3-aryl-5-aryl-[1,2,4]-oxadiazole; and (b) determining whether the compound binds to the antibody; wherein compounds which bind the antibody are potentially therapeutic anticancer compounds.

In another embodiment, the invention pertains to a method of prognosing the efficacy of an anti-cancer TIPRAIP binding composition in a cancer patient comprising: (a) taking a fluid or tissue sample from an individual manifesting a cancer; (b) quantifying the total mRNA encoding TIPRAIP; (c) calculating a ratio comprising the quantity of the mRNA to the average quantity of the mRNA in a population not manifesting the cancer; wherein a ratio greater than 1 indicates that the anti-cancer TIPRAIP binding composition is efficacious.

In another embodiment, the invention pertains to a method of prognosing the efficacy of an anti-cancer TIPRAIP binding composition in a cancer patient comprising: (a)taking a fluid or tissue sample from an individual manifesting a cancer; (b) quantifying the TIPRAIP present in the sample; (c) calculating a ratio comprising the quantity of the TIPRAIP to the average quantity of the TIPRAIP in a population not manifesting the cancer; wherein a ratio greater than 1 indicates that the anti-cancer TIPRAIP binding composition is efficacious.

The invention also relates to the use of the structures of TIPRAIPs to design compositions that bind these polypeptides, or to design compositions that activate apoptosis.

Apoptosis may be induced by the compounds of the present invention within 24 to 48, 24-72 or 24-96 hours of introduction to the cell, or administration to an animal. Apoptosis may also be induced by such compounds from 12 to 36 hours. These compounds preferably have a molecular weight ranging from 200 Daltons (g/mole) to 20,000 Daltons (g/mole). The compounds may also have a molecular weight ranging from 250 Daltons to 10,000 Daltons.

The invention also relates to a complex, comprising: i) a TIPRAIP; and ii) a TIPRAIP binding compound; with the proviso that the TIPRAIP binding compound is not 3-(4-azidophenyl)-5-(3-chloro-thiophen-2-yl)-[1,2,4]-oxadiazole or a substituted 3-aryl-5-aryl-[1,2,4]-oxadiazole.

The invention also relates to a detectably labeled 3-(4-azidophenyl)-5-(3-chloro-thiophen-2-yl)-[1,2,4]-oxadiazole or substituted 3-aryl-5-aryl-[1,2,4]-oxadiazole comprising i) 3-(4-azidophenyl)-5-(3-chloro-thiophen-2-yl)-[1,2,4]-oxadiazole or a substituted 3-aryl-5-aryl-[1,2,4]-oxadiazole; ii) optionally a linker; and iii) a label; wherein the 3-(4-azidophenyl)-5-(3-chloro-thiophen-2-yl)-[1,2,4]-oxadiazole or substituted 3-aryl-5-aryl-[1,2,4]-oxadiazole is covalently linked to the label optionally via the linker. The detectable label may be biotin, a fluorescent label, or a radiolabel.

The invention also relates to a composition comprising i) 3-(4-azidophenyl)-5-(3-chloro-thiophen-2-yl)-[1,2,4]-oxadiazole or a substituted 3-aryl-5-aryl-[1,2,4]-oxadiazole; ii) optionally a linker; and iii) a solid phase; wherein the 3-(4-azidophenyl)-5-(3-chloro-thiophen-2-yl)-[1,2,4]-oxadiazole or substituted 3-aryl-5-aryl-[1,2,4]-oxadiazole is covalently linked to the solid phase optionally via the linker. The solid phase may be agarose or N-hydroxysuccinimidylcarboxyl-agarose.

The invention also relates to a method of treating, preventing or ameliorating a disease responsive to induction of the caspase cascade in an animal, comprising administering to the animal a compound which
i) increases the level of cellular mRNA encoding transforming growth factor beta, cyclin-dependent kinase inhibitor 1A, insulin-like growth factor 2 receptor, or insulin-like growth factor binding protein 3; or
ii) decreases the level of cellular mRNA encoding cyclin D1; with the proviso that the compound is not 3-(4-azidophenyl)-5-(3-chloro-thiophen-2-yl)-[1,2,4]-oxadiazole or a substituted 3-aryl-5-aryl-[1,2,4]-oxadiazole.

The invention also relates to a method of identifying potentially therapeutic anticancer compounds comprising:
(a) contacting cells with one or more test compounds; and
(b) monitoring
   i) cellular increases in mRNA encoding transforming growth factor beta, cyclin-dependent kinase inhibitor 1A, insulin-like growth factor 2 receptor, or insulin-like growth factor binding protein 3; or
   ii) cellular decreases in mRNA encoding cyclin D1;
      wherein test compounds that cause the increases or decreases are potentially therapeutic anticancer compounds; with the proviso that the compounds do not include 3-(4-azidophenyl)-5-(3-chloro-thiophen-2-yl)-[1,2,4]-oxadiazole or a substituted 3-aryl-5-aryl-[1,2,4]-oxadiazole.

The invention also relates to a method of treating, preventing or ameliorating a disease responsive to induction of the caspase cascade in an animal, comprising administering to the animal a compound which interferes with or prevents the binding of TIP-47 to insulin-like growth factor 2 receptor; with the proviso that the compound is not 3-(4-azidophenyl)-5-(3-chloro-thiophen-2-yl)-[1,2,4]-oxadiazole or a substituted 3-aryl-5-aryl-[1,2,4]-oxadiazole.

The invention also relates to a method of identifying potentially therapeutic anticancer compounds comprising monitoring whether one or more test compounds interfere with or prevent the binding of TIP-47 to insulin-like growth factor 2 receptor; wherein test compounds that interfere or prevent the binding are potentially therapeutic anticancer compounds; with the proviso that the compounds do not include 3-(4-azidophenyl)-5-(3-chloro-thiophen-2-yl)-[1,2,4]-oxadiazole or a substituted 3-aryl-5-aryl-[1,2,4]-oxadiazole.

### BRIEF DESCRIPTION OF THE DRAWINGS

FIG. 1A: 3-(3,5-Ditritium-4-azidophenyl)-5-(3-chloro-thiophen-2-yl)-[1,2,4]-oxadiazole (Example 3) Binding to GST-Tip47 immobilized on α-GST-Protein A Sepharose. 2 μM 3-(3,5-ditritium-4-azidophenyl)-5-(3-chloro-thiophen-2-yl)-[1,2,4]-oxadiazole (Example 3) was added to either Protein A Sepharose only, Protein A Sepharose plus anti-GST antibody, anti-GST/Protein A Sepharose plus GST only, or anti-GST/Protein A Sepharose plus GST-Tip47. After TBS washes, eluate was counted on a scintillation counter.

FIG. 1B: 3-(3,5-Ditritium-4-azidophenyl)-5-(3-chloro-thiophen-2-yl)-[1,2,4]-oxadiazole (Example 3) Binding to immunoprecipitated Tip47 from cell lysates. T47D cytosol was labeled with 20 nM 3-(3,5-ditritium-4-azidophenyl)-5-(3-chloro-thiophen-2-yl)-[1,2,4]-oxadiazole (Example 3) and immunoprecipitated with anti-fibronectin (as a control) or anti-Tip47. The immunoprecipitated complex was subject to SDS-PAGE and autoradiography.

FIG. 2: The effect of 5-(3-chlorothiophen-2-yl)-3-(5-chloro-pyridin-2-yl)-[1,2,4]-oxadiazole on mRNA levels of genes of interest. T47D cells were treated for 18 h with 5 μM of 5-(3-chlorothiophen-2-yl)-3-(5-chloro-pyridin-2-yl)-[1,2,4]-oxadiazole or DMSO and total RNA was then isolated. mRNA levels of TGFbeta, p21, cyclin D1, IGF2R, and IGFBP3 were quantitated using realtime PCR as fold change of treatment/control.

FIG. 3A: Realtime PCR showing the down-regulation of the Tip47 at the mRNA level. T47D cells were transfected for 48 h as untransfected, lipid alone, cyclophilin (cph) (100 nM), and Tip47 siRNA (100 nM). Tip47 mRNA levels were normalized to cyclophilin, a housekeeping gene. Cyclophilin downregulation was normalized to GAPD (glyceraldehye phosphate dehydrogenase).

FIG. 3B: Realtime PCR showing the effects of Tip47 downregulation on other genes of interest. T47D cells were transfected for 48 h as untransfected, lipid alone, cyclophilin (cph) (100 nM), and Tip47 siRNA (100 nM). Tip47, cyclin D1, and p21 mRNA levels were normalized to cyclophilin, a housekeeping gene. Cyclophilin downregulation was normalized to GAPD.

FIG. 3C: Western blot representing the down-regulation of Tip47 in siRNA transfected cells and its effect on genes of interest in the presence of compound. T47D cells were transfected with Tip47 siRNA (100 nM) or lipid alone for 48 h. Transfected cells were treated with DMSO or 5-(3-chlorothiophen-2-yl)-3-(5-chloro-pyridin-2-yl)-[1,2,4]-oxadiazole (0.5 μM, compound A) for 6 h. Whole cell lysates of T47D cells post transfection were subjected to SDS-PAGE and immunoblotted onto PVDF. Antibodies against Tip47, p21, and cyclin D1 were used to detect changes in the respective protein ± compound (upper panel). Equal loading was confirmed by western blotting of actin (lower panel).

### DETAILED DESCRIPTION OF THE INVENTION

I. Definitions

Unless defined otherwise, all technical and scientific terms used herein have the same meaning as is commonly understood by one of ordinary skill in the art to which this invention belongs.

As used herein, apoptosis is a highly conserved, genetically programmed form of cellular suicide characterized by distinct morphological changes such as cytoskeletal disruption, cell shrinkage, membrane blebbing, nuclear condensation, fragmentation of DNA, and loss of mitochondrial function.

As used herein, a caspase is a cysteine protease of the interleukin-1β/CED-3 family. As used herein, the caspase cascade is a sequential activation of at least two caspases, or the activation of caspase activity that behaves as if it involves the sequential activation of at least two caspases.

As used herein, “Tail Interacting Protein Related Apoptosis Inducing Protein” and “TIPRAIP” are used interchangeably and refer to SEQ ID NO.: 7, its mutants, homologs, derivatives and fragments which affect apoptosis upon binding 3-(4-azidophenyl)-5-(3-chloro-thiophen-2-yl)-[1,2,4]-oxadiazole or a substituted 3-aryl-5-aryl-[1,2,4]-oxadiazole such as those described herein or in nonprovisional U.S. patent application Ser. No. 10/164,705, filed Jun. 10, 2002 (Cai et al.); or in provisional U.S. Patent Application No. 60/433,953, filed Dec. 18, 2002 (Cai et al.). Methods for determining whether a given TIPRAIP binds to 3-(4-azidophenyl)-5-(3-chloro-thiophen-2-yl)-[1,2,4]-oxadiazole or a substituted 3-aryl-5-aryl-[1,2,4]-oxadiazole can be determined by the assays described herein. As used herein, the term “TIPRAIP binding compound” refers to a compound which binds specifically to an TIPRAIP, induces activation of the caspase cascade, and can be administered in the method of treating, preventing or ameliorating a disease responsive to induction of the caspase cascade in an animal, such as a hyperproliferative disease. As used herein, the term “test compound” refers to a compound that can be tested for its ability to bind TIPRAIP. Test compounds identified as capable of binding TIPRAIP are TIPRAIP binding compounds.

The test compounds may be pure substances or mixtures of substances such as in combinatorial libraries. The test compounds may be any natural product, synthesized organic or inorganic molecule, or biological macromolecules. Preferably, the test compounds are preselected to have <500 MW, ≦5 H-bond donors, ≦10 H-bond acceptors, and logP<5. Computer programs may be used to diversify the compound library. The test compounds may be at least 85% pure.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, may be a mixture of stereoisomers. In such instances, further purification might increase the specific activity of the compound.

As used herein, a disease which is “responsive to induction of the caspase cascade” is a disease which may be treated with an TIPRAIP binding compound. Non-limiting examples of such diseases include hyperproliferative and inflammatory diseases. As used herein, hyperproliferative diseases include any disease characterized by inappropriate cell proliferation. Such hyperproliferative diseases include skin diseases such as psoriasis, as well as cancer. Non limiting examples of inflammatory diseases include autoimmune diseases such as rheumatoid arthritis, multiple sclerosis, insulin-dependent diabetes mellitus, lupus and muscular dystrophy.

As used herein, a cell which expresses a cancer phenotype includes cells which are characteristic of cancer. Such cells may have come from animals manifesting a cancer, from animal bone, tissue or fluid manifesting a cancer, or from cancer cell lines well known in the art.

As used herein, cancer is a group of diseases characterized by the uncontrolled growth and spread of abnormal cells or one in which compounds that activate the caspase cascade have therapeutic use. Such diseases include, but are not limited to, Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical carcinomas, cervical hyperplasia, renal cell carcinomas, endometrial carcinomas, polycythemia vera, essential thrombocytosis, adrenal cortex carcinomas, skin cancer, and prostatic carcinomas.

As used herein an effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

As used herein, treatment means any manner in which the symptoms of a condition, disorder or disease are ameliorated or otherwise beneficially altered.

As used herein, amelioration of the symptoms of a particular disorder by administration of a particular pharmaceutical composition refers to any lessening, whether permanent or temporary, lasting or transient, that can be attributed to or associated with administration of the composition.

As used herein, EC₅₀ refers to a dosage, concentration or amount of a particular compound that elicits a dose-dependent response at 50% of maximal expression of a particular response that is induced, provoked or potentiated by the particular compound.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady, Medicinal Chemistry: A Biochemical Approach, Oxford University Press, New York, pages 388-392 (1985)). For example, succinylsulfathiazole is a prodrug of 4-amino-N-(2-thiazoyl)benzenesulfonamide (sulfathiazole) that exhibits altered transport characteristics.

Examples of prodrugs of the compounds of the invention include the simple esters of carboxylic acid containing compounds (e.g. those obtained by condensation with a C₁₋₄ alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions, and mixtures.

3-(4-Azidophenyl)-5-(3-chloro-thiophen-2-yl)-[1,2,4]-oxadiazole and substituted 3-aryl-5-aryl-[1,2,4]-oxadiazole include those compounds described herein or in nonprovisional U.S. patent application Ser. No. 10/164,705, filed Jun. 10, 2002 (Cai et al.); or in provisional U.S. Patent Application No. 60/433,953, filed Dec. 18, 2002 (Cai et al.).

3-(4-Azidophenyl)-5-(3-chloro-thiophen-2-yl)-[1,2,4]-oxadiazole and substituted 3-aryl-5-aryl-[1,2,4]-oxadiazoles include those represented by Formula I: 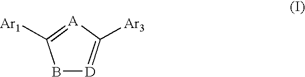
or pharmaceutically acceptable salts or prodrugs or tautomers thereof, wherein:
Ar₁ is optionally substituted aryl or optionally substituted heteroaryl;
Ar₃ is optionally substituted and selected from the group consisting of arylalkyl, aryloxy, phenoxymethyl, anilino, benzylamino, benzylideneamino, benzoylamino and Ar₂, wherein Ar₂ is optionally substituted aryl or optionally substituted heteroaryl; and
A, B and D independently are C, CR₁₀, C(R₁₀)R₁₁, N, NR₁₂, O or S, wherein R₁₀ and R₁₁ are at each occurrence independently hydrogen, optionally substituted alkyl, optionally substituted cycloalkyl or optionally substituted aryl and R₁₂ is at each occurrence independently hydrogen, optionally substituted alkyl, optionally substituted cycloalkyl or optionally substituted aryl, provided that valency rules are not violated. Preferably, R₁₀, R₁₁, and R₁₂ are hydrogen, alkyl, cycloalkyl or aryl; more preferably, R₁₀, R₁₁ and R₁₂ are hydrogen, alkyl or cycloalkyl.

3-(4-Azidophenyl)-5-(3-chloro-thiophen-2-yl)-[1,2,4]-oxadiazole or substituted 3-aryl-5-aryl-[1,2,4]-oxadiazoles also include, without limitation:
3-(4-Azidophenyl)-5-(3-chloro-thiophen-2-yl)-[1,2,4]-oxadiazole;
5-(3-Chloro-thiophen-2-yl)-3-(4-trifluoromethyl-phenyl)-[1,2,4]-oxadiazole;
5-(1-Phenyl-5-trifluoromethyl-1H-pyrazol-4-yl)-3-[3,5-bis(trifluoromethyl)phenyl]-[1,2,4]-oxadiazole;
5-[1-(4-Chloro-phenyl)-5-trifluoromethyl-1H-pyrazol-4-yl]-3-[3,5-bis(trifluoromethyl)phenyl]-[1,2,4]-oxadiazole;
5-(4-Bromo-1-ethyl-3-methyl-1H-pyrazol-5-yl)-3-(5-trifluoromethyl-pyridin-2-yl)-[1,2,4]-oxadiazole;
5-(2-Methy-pyrrol-3-yl)-3-(pyridin-3-yl)-[1,2,4]-oxadiazole;
5-(3-Chloro-thiophen-2-yl)-3-[3,5-bis(trifluoromethyl)phenyl]-[1,2,4]-oxadiazole;
5-(3-Chloro-thiophen-2-yl)-3-(4-chloro-phenyl)-[1,2,4]-oxadiazole;
5-(4-Bromo-3-methoxy-thiophen-2-yl)-3 -(4-trifluoromethyl-phenyl)-[1,2,4]-oxadiazole;
5-(3-Methyl-5-triflurormethyl-isoxazol-4-yl)-3-phenyl-[1,2,4]-oxadiazole;
3-(4-Amino-3,5-dichloro-phenyl)-5-(thiophen-2-yl)-[1,2,4]-oxadiazole;
3-(4-Methyl-phenyl)-5-(thiophen-2-yl)-[1,2,4]-oxadiazole;
5-(3-Chloro-thiophen-2-yl)-3-(2,4-dichloro-phenyl)-[1,2,4]-oxadiazole;
5-(3-Chloro-thiophen-2-yl)-3-(4-(methylsulphonylamino)phenyl)-[1,2,4]-oxadiazole;
5-(3-Chloro-thiophen-2-yl)-3-(4-methyl-phenyl)-[1,2,4]-oxadiazole;
5-(3-Chloro-thiophen-2-yl)-3-(4-fluoro-phenyl)-[1,2,4]-oxadiazole;
5-(3-Chloro-thiophen-2-yl)-3-(4-nitro-phenyl)-[1,2,4]-oxadiazole;
5-(3-Chloro-thiophen-2-yl)-3-phenyl-[1,2,4]-oxadiazole;
5-(3-Chloro-thiophen-2-yl)-3-(4-trifluoromethoxy-phenyl)-[1,2,4]-oxadiazole;
5-(3-Chloro-thiophen-2-yl)-3-(4-methoxy-phenyl)-[1,2,4]-oxadiazole;
5-(3-Chloro-thiophen-2-yl)-3-(3,4-methylenedioxy-phenyl)-[1,2,4]-oxadiazole;
5-(3-Bromo-thiophen-2-yl)-3-(4-chloro-phenyl)-[1,2,4]-oxadiazole;
5-(3-Chloro-thiophen-2-yl)-3-(pyridin-4-yl)-[1,2,4]-oxadiazole;
5-(3-Chloro-thiophen-2-yl)-3-(4-dimethylamino-phenyl)-[1,2,4]-oxadiazole;
5-(3-Chloro-thiophen-2-yl)-3-(pyridin-3-yl)-[1,2,4]-oxadiazole;
5-(3-Chloro-thiophen-2-yl)-3-(pyridin-2-yl)-[1,2,4]-oxadiazole;
5-(3-Chloro-thiophen-2-yl)-3-(4-hydroxy-phenyl)-[1,2,4]-oxadiazole;
5-(3-Chloro-thiophen-2-yl)-3-(N-oxide-pyridin-4-yl-)-[1,2,4]-oxadiazole;
5-(3-Methyl-furan-2-yl)-3-(4-chloro-phenyl)-[1,2,4]-oxadiazole;
5-(3 -Methyl-furan-2-yl)-3-(5 -trifluoromethyl-pyridin-2-yl)-[1,2,4]-oxadiazole;
3-(4-Chloro-phenyl)-5-(3-methyl-thiophen-2-yl)-[1,2,4]-oxadiazole;
5-(3-Bromo-furan-2-yl)-3-(4-chloro-phenyl)-[1,2,4]-oxadiazole;
5-(3-Bromo-furan-2-yl)-3-(4-trifluoromethyl-phenyl)-[1,2,4]-oxadiazole;
5-(3-Chloro-thiophen-2-yl)-3-(4-chloro-benzyl)-[1,2,4]-oxadiazole;
5-(4-Chloro-1H-pyrazol-3-yl)-3-(4-chloro-phenyl)-[1,2,4]-oxadiazole;
5-(4-Chloro-1H-pyrazol-3-yl)-3-(5-trifluoromethyl-pyridin-2-yl)-[1,2,4]-oxadiazole;
5-(3-Chloro-furan-2-yl)-3-(4-chloro-phenyl)-[1,2,4]-oxadiazole;
5-(3-Chloro-furan-2-yl)-3-(5-trifluoromethyl-pyridin-2-yl)-[1,2,4]-oxadiazole;
(4-Chloro-benzylidene)-[5-(3-chloro-thiophen-2-yl)-[1,2,4]-oxadiazol-3-yl]-amine:
[5-(3-Chloro-thiophen-2-yl)-[1,2,4]-oxadiazol-3-yl]-(3-trifluoromethyl-benzylidene)-amine;
3-(4-Amino-phenyl)-5-(3-chloro-thiophen-2-yl)-[1,2,4]-oxadiazole;
3-(4-Azido-phenyl)-5-(3-chloro-thiophen-2-yl)-[1,2,4]-oxadiazole;
5-(3-Chloro-thiophen-2-yl)-3-(4-trifluoromethyl-phenyl)-[1,3,4]-oxadiazole;
5-(4-Chloro-thiazol-5-yl)-3-(5-chloro-pyridin-2-yl)-[1,2,4]-oxadiazole;
5-(3-Chloro-thiophen-2-yl)-3-(5-chloro-pyridin-2-yl)-[1,2,4]-oxadiazole;
3-(4-Amino-pyrimidin-5-yl)-5-(3-chloro-thiophen-2-yl)-[1,2,4]-oxadiazole;
5-(3-Chloro-thiophen-2-yl)-3-(5-trifluoromethyl-pyridin-2-yl)-[1,2,4]-oxadiazole;
5-(3-Bromo-5-formyl-furan-2-yl)-3-(4-chloro-phenyl)-[1,2,4]-oxadiazole;
5-(3-Chloro-thiophen-2-yl)-3-(pyrimidin-2-yl)-[1,2,4]-oxadiazole;
5-(3-Chloro-thiophen-2-yl)-3-(N-oxide-pyridin-3-yl)-[1,2,4]-oxadiazole;
5-(3-Chloro-thiophen-2-yl)-3-(6-chloro-pyridin-3-yl)-[1,2,4]-oxadiazole;
5-(3-Chloro-thiophen-2-yl)-3-(4-chloro-3-trifluoromethyl-phenyl)-[1,2,4]-oxadiazole;
5-(3-Chloro-thiophen-2-yl)-3-(3,4-dichloro-phenyl)-[1,2,4]-oxadiazole;
5-(3-Chloro-thiophen-2-yl)-3-(5-trifluoromethyl-pyridin-2-yl)-[1,2,4]-oxadiazole;
3-(3-Bromo-thiophen-2-yl)-5-(4-chloro-phenyl)-[1,2,4]-oxadiazole;
3-(3-Bromo-thiophen-2-yl)-5-(4-trifluoromethyl-phenyl)-[1,2,4]-oxadiazole;
3-(4-Acetamido-phenyl)-5-(3-chloro-thiophen-2-yl)-[1,2,4]-oxadiazole;
5-(3-Chloro-thiophen-2-yl)-3-(3-trifluoromethyl-phenyl)-[1,2,4]-oxadiazole;
5-(3-Chloro-thiophen-2-yl)-3-(6-trifluoromethyl-pyridin-3-yl)-[1,2,4]-oxadiazole;
3-(2-Amino-4-chloro-phenyl)-5-(3-chloro-thiophen-2-yl)-[1,2,4]-oxadiazole;
5-(3-Chloro-thiophen-2-yl)-3-(quinoline-2-yl)-[1,2,4]-oxadiazole;
5-(3-Chloro-thiophen-2-yl)-3-(isoquinoline-3-yl)-[1,2,4]-oxadiazole;
5-(3-Chloro-thiophen-2-yl)-3-(4-methyl-pyridin-2-yl)-[1,2,4]-oxadiazole;
3-(4-Chloro-phenyl)-5-(2-methyl-4-trifluoromethyl-thiazol-5-yl)-[1,2,4]-oxadiazole;
5-(3-Chloro-thiophen-2-yl)-3-(4-cyano-pyridin-2-yl)-[1,2,4]-oxadiazole;
5-(3-Chloro-thiophen-2-yl)-3-(4-cyano-phenyl)-[1,2,4]-oxadiazole;
5-(3-Chloro-thiophen-2-yl)-3-(5-methyl-pyridin-2-yl)-[1,2,4]-oxadiazole;
5-(3-Chloro-thiophen-2-yl)-3-(6-methyl-pyridin-3-yl)-[1,2,4]-oxadiazole;
5-(3-Chloro-thiophen-2-yl)-3-(pyrazin-2-yl)-[1,2,4]-oxadiazole;
5-(3-Chloro-thiophen-2-yl)-3-[4-(methyl carboxy)-phenyl]-[1,2,4]-oxadiazole;
5-(3-Chloro-thiophen-2-yl)-3-(quinolin-3-yl)-[1,2,4]-oxadiazole;
5-(3-Chloro-thiophen-2-yl)-3-(8-hydroxy-quinolin-2-yl)-[1,2,4]-oxadiazole;
5-(3-Cyano-thiophen-2-yl)-3-(5-trifluoromethyl-pyridin-2-yl)-[1,2,4]-oxadiazole;
5-(3-Chloro-thiophen-2-yl)-3-(5,6-dichloro-pyridin-3-yl)-[1,2,4]-oxadiazole;
5-(3-Bromo-furan-2-yl)-3-(5-chloro-pyridin-2-yl)-[1,2,4]-oxadiazole;
5-(3-Bromo-furan-2-yl)-3-(6-trifluoromethyl-pyridin-3-yl)-[1,2,4]-oxadiazole;
5-(3-Bromo-furan-2-yl)-3-(5-trifluoromethyl-pyridin-2-yl)-[1,2,4]-oxadiazole;
5-(3-Chloro-thiophen-2-yl)-3-(2-methyl-thiazol-4-yl)-[1,2,4]-oxadiazole;
5-(3-Chloro-thiophen-2-yl)-3-(5-nitro-thiazol-2-yl)-[1,2,4]-oxadiazole;
5-(3-Chloro-thiophen-2-yl)-3-(7-methyl-5-trifluoromethyl-pyrazolo[1,5-a]pyrimidin-3-yl)-[1,2,4]-oxadiazole;
5-(3-Bromo-furan-2-yl)-3-(4-chloro-phenyl)-[1,2,4]-oxadiazole;
5-(3-Chloro-thiophen-2-yl)-3-[2-(4-chloro-phenyl)-ethyl]-[1,2,4]-oxadiazole;
5-(3-Chloro-thiophen-2-yl)-3-(4-chloro-phenoxymethyl)-[1,2,4]-oxadiazole;
5-(3-Chloro-thiophen-2-yl)-2-(4-trifluoromethoxy-phenyl)-1H-imidazole;
5-(3-Bromo-thiophen-2-yl)-2-(4-trifluoromethyl-phenyl)-1H-imidazole;
5-(3-Chloro-thiophen-2-yl)-2-(4-trifluoromethyl-phenyl)-1H-imidazole;
5-(6-Chloro-pyridin-3-yl)-2-(3-chloro-thiophen-2-yl)-[1,3,4]-oxadiazole;
2-(3-Chloro-thiophen-2-yl)-5-(pyridin-3-yl)-[1,3,4]-oxadiazole;
5-(4-Chloro-phenyl)-2-(3-chloro-thiophen-2-yl)-[1,3,4]-oxadiazole;
5-(3-Bromo-5-morpholinomethyl-furan-2-yl)-3-(4-chloro-phenyl)-[1,2,4]-oxadiazole;
5-(3-Bromo-5-hydroxymethyl-furan-2-yl)-3-(4-chloro-phenyl)-[1,2,4]-oxadiazole;
5-(3-Chloro-thiophen-2-yl)-3-(4-trifluoromethyl-phenyl)-1H-[1,2,4]-triazole;
5-(3-Chloro-thiophen-2-yl)-3-phenyl-1H-[1,2,4]-triazole;
5-(3-Chloro-thiophen-2-yl)-3-(4-methyl-phenyl)-1H-[1,2,4]-triazole;
5-(3-Chloro-thiophen-2-yl)-3-(3-methyl-phenyl)-1H-[1,2,4]-triazole;
5-(3-Chloro-thiophen-2-yl)-3-(pyridin-2-yl)-1H-[1,2,4]-triazole;
2-(3-Chloro-thiophen-2-yl)-5-phenyl-oxazole;
5-(4-Bromo-phenyl)-2-(3-chloro-thiophen-2-yl)-oxazole;
2-(3-Chloro-thiophen-2-yl)-5-(4-methoxy-phenyl)-oxazole;
5-(4-Chloro-phenyl)-2-(3-chloro-thiophen-2-yl)-oxazole;
5-(3-Chloro-thiophen-2-yl)-2-phenyl-oxazole;
2-(4-Chloro-phenyl)-5-(3-chloro-thiophen-2-yl)-oxazole;
2-(6-Chloro-pyridin-3-yl)-5-(3-chloro-thiophen-2-yl)-oxazole;
5-(3-Chloro-thiophen-2-yl)-2-(4-trifluoromethyl-phenyl)-oxazole;
2-(3-Chloro-thiophen-2-yl)-4-(4-trifluoromethyl-phenyl)-oxazole;
4-(4-Chloro-phenyl)-2-(3-chloro-thiophen-2-yl)-oxazole;
3-(4-Chloro-phenyl)-5-(3-chloro-thiophen-2-yl)-1H-pyrazole;
4-Chloro-N-[5-(3-chloro-thiophen-2-yl)-[1,2,4]-oxadiazol-3-yl]-benzamide;
5-(4-Chloro-phenyl)-3-(3-chloro-thiophen-2-yl)-1-phenyl-1H-pyrazole;
5-(4-Chloro-phenyl)-3-(3-chloro-thiophen-2-yl)-1-methyl-1H-pyrazole;
5-(4-Chloro-phenyl)-1-(3-chloro-phenyl)-3-(3-chloro-thiophen-2-yl)-1H-pyrazole;
1,5-Bis-(4-chloro-phenyl)-3-(3-chloro-thiophen-2-yl)-1H-pyrazole;
5-(4-Chloro-phenyl)-3-(3-chloro-thiophen-2-yl)-1-(pyridin-2-yl)-1H-pyrazole;
5-(4-Chloro-phenyl)-3-(3-chloro-thiophen-2-yl)-1-(4-carboxy-phenyl)-1H-pyrazole;
5-(4-Chloro-phenyl)-3-(3-chloro-thiophen-2-yl)-1-(4-methanesulfonyl-phenyl)-1H-pyrazole;
5-(4-Chloro-phenyl)-3-(3-chloro-thiophen-2-yl)-1-(2-hydroxyethyl)-1H-pyrazole;
5-(3-Chloro-thiophen-2-yl)-3-(4-chloro-anilino)[1,2,4]-oxadiazole;
5-(3-Bromo-furan-2-yl)-3-(4-fluoro-phenyl)-[1,2,4]-oxadiazole;
5-(3-Chloro-furan-2-yl)-3-(5-chloro-pyridin-2-yl)-[1,2,4]-oxadiazole;
5-(3-Chloro-furan-2-yl)-3-(4-trifluoromethyl-phenyl)-[1,2,4]-oxadiazole;
5-(1-Chloro-furan-2-yl)-3-(4-chloro-phenyl)-[1,2,4]-oxadiazole;
5-(3-Bromo-furan-2-yl)-3-(5-trifluoromethyl-pyridin-2-yl)-[1,2,4]-oxadiazole;
5-(3-Bromo-furan-2-yl)-3-(5-chloro-pyridin-2-yl)-[1,2,4]-oxadiazole;
4-(2-{4-[5-(3-Chloro-thiophen-2-yl)-[1,2,4]-oxadiazol-3-yl]-phenoxy}-ethyl)-morpholine;
(2-{4-[5-(3-Chloro-thiophen-2-yl)-[1,2,4]-oxadiazol-3-yl]-phenoxy}-ethyl)-dimethylamine;
{4-[5-(3-Chloro-thiophen-2-yl)-[1,2,4]-oxadiazol-3-yl]-phenoxy}-acetic acid methyl ester;
5-(3,4,5-Trichloro-thiophen-2-yl)-3-(4-trifluoromethyl-phenyl)-[1,2,4]-oxadiazole;
5-(3-Chloro-thiophen-2-yl)-3-(6-methoxy-pyridin-3-yl)-[1,2,4]-oxadiazole;
3-(4-Butoxy-phenyl)-5-(3-chloro-thiophen-2-yl)-[1,2,4]-oxadiazole; and
3-(4-Amino-3,5-diiodo-phenyl)-5-(3-chloro-thiophen-2-yl)-[1,2,4]-oxadiazole;
and pharmaceutically acceptable salts or prodrugs thereof.

3-(4-Azidophenyl)-5-(3-chloro-thiophen-2-yl)-[1,2,4]-oxadiazole and substituted 3-aryl-5-aryl-[1,2,4]-oxadiazoles also include compounds represented by Formula II: 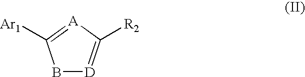
or pharmaceutically acceptable salts or prodrugs or tautomers thereof, wherein:
Ar₁ is optionally substituted aryl or optionally substituted heteroaryl;
R₂ is optionally substituted and selected from the group consisting of arylalkyl, arylalkenyl, aryloxy, arylalkyloxy, phenoxymethyl, anilino, benzylamino, benzylideneamino, benzoylamino, heterocycle, carbocycle and Ar₂, wherein Ar₂ is optionally substituted aryl or optionally substituted heteroaryl; and
A, B and D independently are C, CR₁₀, C(R₁₀)R₁₁, N, NR₁₂, O or S, wherein R₁₀ and R₁₁, are at each occurrence independently hydrogen, optionally substituted alkyl, optionally substituted cycloalkyl or optionally substituted aryl and R₁₂ is at each occurrence independently hydrogen, optionally substituted alkyl, optionally substituted cycloalkyl or optionally substituted aryl, provided that valency rules are not violated.

3-(4-Azidophenyl)-5-(3-chloro-thiophen-2-yl)-[1,2,4]-oxadiazole or substituted 3-aryl-5-aryl-[1,2,4]-oxadiazoles also include, without limitation, the following:
3-(3-Amino-4-chloro-phenyl)-5-(3-chlorothiophen-2-yl)-[1,2,4]-oxadiazole;
5-(3-Chlorothiophen-2-yl)-3-(3-dimethylamino-4-chloro-phenyl)-[1,2,4]-oxadiazole;
3-(3-Amino-4-chloro-phenyl)-5-(3 -bromofuran-2-yl)-[1,2,4]-oxadiazole;
5-(3-Bromofuran-2-yl)-3-(3-dimethylamino-4-chloro-phenyl)-[1,2,4]-oxadiazole;
N-{2-Chloro-5-[5-(3-chloro-thiophen-2-yl)-[1,2,4]-oxadiazol-3-yl]-phenyl}-2-(4-methyl-piperazin-1-yl)-acetamide;
N-{2-Chloro-5-[5-(3-chloro-thiophen-2-yl)-[1,2,4]-oxadiazol-3-yl]-phenyl}-succinamic acid ethyl ester;
5-(3-Chlorothiophen-2-yl)-3-(4-chloro-3-cyano-phenyl)-[1,2,4]-oxadiazole;
3-(4-Chloro-benzyloxy)-5-(3-chloro-thiophen-2-yl)-[1,2,4]-oxadiazole;
5-(3-Chlorothiophen-2-yl)-3-(4-chloro-3-fluoro-phenyl)-[1,2,4]-oxadiazole;
5-(3-Chlorothiophen-2-yl)-3-(4-chloro-3-nitro-phenyl)-[1,2,4]-oxadiazole;
3-(5-Chloro-pyridin-2-yl)-5-(3-methoxy-thiophen-2-yl)-[1,2,4]-oxadiazole;
3-(5-Chloro-pyridin-2-yl)-5-(3-methyl-3H-imidazol-4-yl)-[1,2,4]-oxadiazole;
3-[2-(4-Chloro-phenyl)-vinyl]-5-(3-chloro-thiophen-2-yl)-[1,2,4]-oxadiazole;
5-(3-Chloro-1H-pyrrol-2-yl)-3-(5-chloro-pyridin-2-yl)-[1,2,4]-oxadiazole;
3-(4-Chloro-phenyl)-5-(3-chloro-1H-pyrrol-2-yl)-[1,2,4]-oxadiazole;
5-(3-Chloro-1-methyl-1H-pyrrol-2-yl)-3-(4-chloro-phenyl)-[1,2,4]-oxadiazole;
5-[3-Chloro-1-(2-dimethylaminoethyl)-1H-pyrrol-2-yl]-3-(4-chloro-phenyl)-[1,2,4]-oxadiazole;
5-(3-Chlorothiophen-2-yl)-3-(1-piperidinyl)-[1,2,4]-oxadiazole; and
5-(3-Chlorothiophen-2-yl)-3-(4-morpholinyl)-[1,2,4]-oxadiazole;
and pharmaceutically acceptable salts or prodrugs thereof.

As used herein in the context of polypeptides, “mutants” include TIPRAIPs given by SEQ ID NO.: 7 having one or more amino acid substitutions. Mutants include naturally occurring or artificially generated TIPRAIPs. Naturally occurring mutants include TIPRAIPs which are encoded by allelic variation in the TIPRAIP gene.

As used herein in the context of polypeptides, “homologs” include TIPRAIP sequences that are 70% or more homologous to SEQ ID NO.: 7, as measured by the percent identity of the homolog's primary amino acid sequence to that of SEQ ID NO.: 7. For example, a homolog that is only 400 amino acids long is 34 amino acids shorter than SEQ ID NO.: 7. However, if 380 amino acids of this homolog have an identical sequential arrangement with respect to SEQ ID NO.: 7, then the homolog is 95% identical ((380/400) ×100%) to SEQ ID NO.: 7. Preferably, homologs are 90%, 91%, 92%, 93%, 94%, 95%, 96%, 97%, 98%, 99% or more homologous to SEQ ID NO.: 7.

As used herein in the context of polypeptides, “derivatives” refer to TIPRAIPs that are derivatized or modified forms of SEQ ID NO.: 7. Derivatives of TIPRAIP may include, for example, post-expression modifications, amidated carboxyl groups, glycosylated amino acid residues, and formylated and acetylated amino groups. Derivatives of TIPRAIP also include TIPRAIP having a leader or secretory sequence, such as a pre-, pro- or prepro-protein sequence; or TIPRAIP fused to amino acids or other proteins, such as those which provide additional functionalities.

As used herein in the context of polypeptides, “fragments” refer to any oligopeptide or polypeptide which is less than the full length of SEQ ID NO.: 7. Fragments may be 70% or more identical to SEQ IID NO.: 7. Preferably, fragments are 90%, 91%, 92%, 93%, 94%, 95%, 96%, 97%, 98%, 99% or more identical to SEQ ID NO.: 7. Fragments may be 20, 25, 30, 40, 50, 60, 70, 80, 90, 100, 120, 140, 160, 180, 200, 300, 400 or more contiguous amino acids of SEQID NO.: 7.

Fragments which are 20 amino acids long (referred to as “20-mers”) include amino acids 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, and 415-434, corresponding to SEQ ID NO.: 7. Fragments also include any combination of two or more overlapping or adjacent 20-mers of the above list of 20-mers. For example, a combination of amino acids 243-262 of SEQ ID NO.: 7 and amino acids 255-274 of SEQ ID NO.: 7 provides a fragment that is 32 amino acids long (a 32-mer) composed of amino acids 243-274 of SEQ ID NO.: 7.

As used herein, “nucleotides” and “polynucleotides” are used interchangeably and refer to single or double stranded polynucleic acid molecules composed of DNA or RNA. The term “nucleotides” includes any polynucleic acid molecule that encodes for SEQ ID NO.: 7, its mutants, homologs, derivatives and fragments which affect apoptosis upon binding 3-(4-azidophenyl)-5-(3-chloro-thiophen-2-yl)-[1,2,4]-oxadiazole or a substituted 3-aryl-5-aryl-[1,2,4]-oxadiazole such as those described herein or in nonprovisional U.S. patent application Ser. No. 10/164,705, filed Jun. 10, 2002 (Cai et al.); or in provisional U.S. Patent Application No. 60/433,953, filed Dec. 18, 2002 (Cai et al.). The term “nucleotides” also includes any polynucleic acid molecule which hybridize to a nucleotide which encodes for SEQ ID NO.: 7, its mutants, homologs, derivatives and fragments which affect apoptosis upon binding 3-(4-azidophenyl)-5-(3-chloro-thiophen-2-yl)-[1,2,4]-oxadiazole or a substituted 3-aryl-5-aryl-[1,2,4]-oxadiazole such as those described herein or in nonprovisional U.S. patent application Ser. No. 10/164,705, filed Jun. 10, 2002 (Cai et al.); or in provisional U.S. Patent Application No. 60/433,953, filed Dec. 18, 2002 (Cai et al.). Nucleotides encoding for TIPRAIPs include the coding sequence for the TIPRAIP polypeptide and optionally additional sequences.

The term “nucleotides” also includes variants. “Variants” refer to one of several alternate forms of a gene occupying a given locus on a chromosome of an organism. Genes II, Lewin, B., ed., John Wiley & Sons, New York (1985). “Variants” also includes non-naturally occurring variants produced using art-known mutagenesis techniques. Variants include those produced by nucleotide substitutions, deletions or additions which may involve one or more nucleotides. The variants may be altered in regions coding for TIPRAIP, other regions or both. Alterations in the coding regions may produce conservative or non-conservative amino acid substitutions, deletions or additions. Silent substitutions, additions and deletions which do not alter the properties and activities of the TIPRAIP or portions thereof, and conservative substitutions may also be used.

The term “nucleotides” also includes splice variants. “Splice variants” refer to a transcribed RNA in which one or more DNA introns are removed. Hence, the skilled artisan will recognize that any of the nucleotides described herein may have a splice variant. TIPRAIPs also include polypeptides encoded by these splice variants.

Nucleotides encoding for TIPRAIPs may include, but are not limited to, those encoding the amino acid sequence of the TIPRAIPs described herein by themselves. Nucleotides encoding for TIPRAIPs also include those encoding TIPRAIP and additional nucleotide sequences. “Additional nucleotide sequences” may include, but are not limited to i) nucleic acid sequences which encode an amino acid leader or secretory sequence, such as a pre-, pro- or prepro-protein sequence; ii) non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; and iii) an additional coding sequence which codes for additional amino acids, such as those which provide additional functionalities. Thus, the nucleotide sequence encoding the TIPRAIP may be fused to a marker sequence, such as a sequence encoding a peptide which facilitates purification of the fused polypeptide. In other embodiments of this aspect of the invention, the marker amino acid sequence is a hexa-histidine peptide, such as the tag provided in a pQE vector (Qiagen, Inc.), among others, many of which are commercially available. As described in Gentz et al, Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al, Cell 37:767-778 (1984).

Nucleotides which encode for TIPRAIP may also comprise polynucleotides which hybridize under stringent hybridization conditions to a portion of the polynucleotides described herein, as described in U.S. Pat. No. 6,027,916. By a polynucleotide which hybridizes to a “portion” of a polynucleotide is intended a polynucleotide (either DNA or RNA) hybridizing to at least about 15, 20, 30, 40, 50, 60 or 70 nucleotides (nt) of the reference polynucleotide. These are useful as diagnostic probes and primers.

Nucleotides are at least 90%, 91%, 92%, 93%, 94%, 95%, 96%, 97%, 98%, 99% or more identical to the sequences described herein. By a polynucleotide having a nucleotide sequence at least, for example, 95% “identical” to a reference nucleotide sequence encoding TIPRAIP, is intended that the nucleotide sequence of the polynucleotide is identical to the reference sequence except that the polynucleotide sequence may include up to five point mutations per each 100 nucleotides of the reference nucleotide sequence encoding the TIPRAIP. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 91%, 92%, 93%, 94%, 95%, 96%, 97%, 98%, 99% or more identical to a nucleotide sequences described herein can be determined conventionally using known computer programs such as the Bestfit program. Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

Of course, due to the degeneracy of the genetic code, one of ordinary skill in the art will immediately recognize that a large number of the nucleic acid molecules having a sequence at least 90%, 91%, 92%, 93%, 94%, 95%, 96%, 97%, 98%, 99% or more identical to the nucleic acid sequences described herein will encode TIPRAIP. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode TIPRAIP. This is because the skilled artisan is fully aware of amino acid substitutions that are either less likely or not likely to significantly effect protein function For example, replacing one aliphatic amino acid with a second aliphatic amino acid is not likely to alter TIPRAIP function. Guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

As used herein, a cell which “up regulates” TIPRAIP is a cell with an elevated level of TIPRAIP as compared to normal cells or cells which down regulate TIPRAIP. The manner by which a cell up regulates TIPRAIP is described below and includes, for example, an altered TIPRAIP gene or TIPRAIP promoter, or a transfection vector that encodes TIPRAIP. As used herein, a cell which “down regulates” TIPRAIP is a cell with a reduced level of TIPRAIP as compared to normal cells or as compared to cells which up regulate TIPRAIP. The manner by which a cell down regulates TIPRAIP is described below and includes, for example, an altered TIPRAIP gene or TIPRAIP promoter, antisense mRNA, or RNAi. As used herein, a “normal” cell neither up regulates or down regulates TIPRAIP. Hence, a normal cell does not have an altered TIPRAIP gene or TIPRAIP promoter, a transfection vector encoding TIPRAIP, antisense mRNA or RNAi. Elevated levels of TIPRAIP include increased levels of functional TIPRAIP. Reduced levels of TIPRAIP includes reduced levels of expressed or reduced levels of functional TIPRAIP. Normal cells have less functional TIPRAIP than cells which up regulate TIPRAIP; and more functional TIPRAIP than cells which down regulate TIPRAIP.

As used herein, a subinducing amount of a substance is an amount that is sufficient to produce a measurable change in caspase cascade activity when used in the method of the present invention and which produces a greater measurable change in caspase cascade activity when used in synergistic combination with an TIPRAIP binding compound in the method of the present invention.

“Label” is used herein to refer to any atom or molecule that is detectable and can be attached to a protein or test compound of interest. Examples of labels include, but are not limited to, radiolabels, fluorescent labels, phosphorescent labels, chemiluminescent labels and magnetic labels. Any label known in the art can be used in the present invention. As used herein, “homogenous assays” refer to assays in which all components are mixed together in the same phase. One example of a homogenous assay is where the components mixed together are all in solution. In contrast, “heterogenous assays” refer to assays in which a first component is attached to a solid phase such as a bead or other solid substrate and one or more additional components are in solution.

As used herein, the term “fluorophore” or “fluorescent group” means any conventional chemical compound, which when excited by light of suitable wavelength, will emit fluorescence with high quantum yield. See, for example, J. R. Lakowicz in “Principles of Fluorescence Spectroscopy,” Plenum Press, 1983. Numerous known fluorophores of a wide variety of structures and characteristics are suitable for use in the practice of this invention. In choosing a fluorophore for fluorescence polarization assays, it is preferred that the lifetime of the fluorophore's exited state be long enough, relative to the rate of motion of the labeled test compound, to permit measurable loss of polarization following emission. Typical fluorescing compounds, which are suitable for use in the present invention, include, for example, rhodamine, substituted rhodamine, fluorescein, fluorescein isothiocyanate, naphthofluorescein, dichlorotriazinylamine fluorescein, dansyl chloride, phycoerythrin, and umbelliferone. Other suitable fluorescent groups for use in the present invention include, but are not limited to, those described in U.S. Pat. Nos. 4,255,329, 4,668,640 and 5,315,015.

As used herein, the term “reporter molecule” is synonymous with the term “reporter compound” and the two terms are used interchangeably. A reporter molecule is a fluorogenic, chromogenic or chemiluminescent substrate that produces a signal such as fluorescence, light absorption within the ultraviolet, visible or infrared spectrum, or light emission, under the influence of the caspase cascade.

The reporter molecule may be composed of at least two covalently linked parts. One part is an amino acid sequence which may be recognized by any of the intracellular proteases or peptidases that are produced as a result of caspase cascade activation. This sequence is bonded to an aromatic or conjugated moiety that undergoes a detectable physical change upon its release from all or part of the amino acid sequence. Such moieties include a fluorogenic moiety that fluoresces more strongly after the reporter molecule is hydrolyzed by one of the proteases, a chromogenic moiety that changes its light absorption characteristics after the reporter molecule is hydrolyzed by one of the proteases, or a chemiluminescent moiety that produces light emission after the reporter molecule is hydrolyzed by one of the proteases. Alternatively, the aromatic or conjugated moiety may be linked to a plurality of aminoacid sequences.

One type of such a reporter molecule is given by Formula III:
x-y-z (III)
or biologically acceptable salts or pro-reporter molecules (such as methyl ester form of carboxyl-containing amino acid residues) thereof, wherein x and z is the same or different and is a peptide or amino acid or acyl group or other structure such that compounds of Formula III are substrates for a caspase or other enzyme involved in the intracellular apoptosis cascade; and wherein the scissile bond is only one or both of the x-y and y-z bonds in Formula III when x is the same as z, or wherein the scissile bond is only one of the x-y or y-z bond in Formula III when x is not the same as z. y is a fluorogenic or fluorescent moiety. See U.S. Pat. No. 6,342,611.

Particular reporter compounds are represented by Formula IV:
R₁-(AA)ₙ-Asp-y-Asp-(AA)ₙ-R₁ (IV)
or biologically acceptable salts or pro-reporter molecules (such as methyl ester form of carboxyl-containing amino acid residues) thereof, wherein R₁ is an N-terminal protecting group such as t-butyloxycarbonyl, acetyl, and benzyloxycarbonyl; each AA independently is a residue of any natural or non-natural α-amino acid or β-amino acid, or derivatives of an α-amino acid or β-amino acid; each n is independently 0-5; and y is a fluorogenic or fluorescent moiety. y may be a Rhodamine including Rhodamine 110, Rhodamine 116 and Rhodamine 19.

Other particular reporter compounds are represented by Formula V: 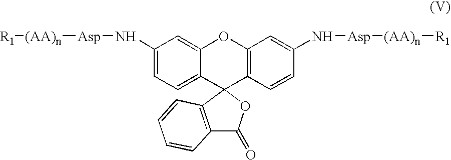
or biologically acceptable salts or pro-reporter molecules (such as methyl ester form of carboxyl-containing amino acid residues) thereof, wherein R₁, AA, n are as defined previously in Formula IV. R₁ may be t-butyloxycarbonyl, acetyl and benzyloxycarbonyl. Values of n are 1-3.

Another group of compounds falling within the scope of Formula III include compounds wherein x is not the same as z. Particular compounds of this group include those wherein x is a peptide or other structure which makes the compound a substrate for a caspase or other enzyme related to apoptosis, and the x-y bond in Formula III is the only bond which is scissile under biological conditions. z is a blocking group and the y-z bond in Formula III is not a scissile bond under biological conditions.

Specifically, the fluorogenic or fluorescent reporter compounds that may be used in this invention are of Formula VI:
R₁-(AA)ₙ-Asp-y—R₆ (VI)
or biologically acceptable salts or pro-reporter molecules (such as methyl ester form of carboxyl-containing amino acid residues) thereof, wherein: R₁, AA, n and y are as defined previously in Formula IV; and R₆ is a blocking group which is not an amino acid or a derivative of an amino acid.

Particular R₆ blocking groups include, but are not limited to, an alkyloxycarbonyl group such as methoxycarbonyl, an arylalkyloxycarbonyl group such as benzyloxycarbonyl, a C₂₋₆ acyl (alkanoyl) group such as acetyl, a carbamyl group such as dimethylcarbamyl, and an alkyl, haloalkyl or aralkyl sulfonyl group such as methanesulfonyl. Particular y is a Rhodamine including Rhodamine 110, Rhodamine 116 and Rhodamine 19.

In other embodiments, the reporter compounds are represented by Formula VII: 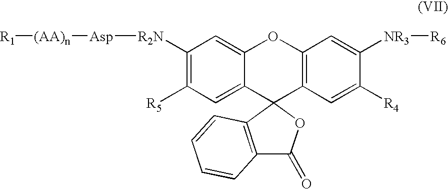
or biologically acceptable salts or pro-reporter molecules (such as methyl ester form of carboxyl-containing amino acid residues) thereof, wherein R₁, R₆, AA and n are as defined previously in Formulae IV and VI; R₂ and R₃ are the same or different and are independently hydrogen, alkyl or aryl; and R₄ and R₅ are the same or different and are independently hydrogen or alkyl.

R₁ may be t-butyloxycarbonyl, acetyl and benzyloxycarbonyl. Values of n may be 1-3. R₂ and R₃ may be hydrogen, methyl or ethyl. R₄ and R₅ may be hydrogen or methyl. R₆ blocking groups include, but are not limited to, an alkyloxycarbonyl group such as methoxycarbonyl, an arylalkyloxycarbonyl group such as benzyloxycarbonyl, an acyl group such as acetyl, a carbamyl group such as dimethylcarbamyl, and an alkyl, haloalkyl or aralkyl sulfonyl group such as methanesulfonyl.

Example of reporter molecules which are useful for the screening methods of the present invention include N-(Ac-DEVD)-N′-acetyl-Rhodamine 110 (SEQ ID NO.: 23), N-(Ac-DEVD)-N′-ethoxycarbonyl-Rhodamine 110 (SEQ ID NO.: 23), N-(Ac-DEVD)-N′-hexyloxycarbonyl-Rhodamine 110 (SEQ ID NO.: 23), N-(Ac-DEVD)-N′-octyloxycarbonyl-Rhodamine 110 (SEQ ID NO.: 23), N-(Ac-DEVD)-N′-decyloxycarbonyl-Rhodamine 110 (SEQ ID NO.: 23), N-(Ac-DEVD)-N′-dodecyloxycarbonyl-Rhodamine 110 (SEQ ID NO.: 23), N-(Ac-DEVD)-N′-2-butoxyethoxycarbonyl-Rhodamine 110 (SEQ ID NO.: 23), N-(Ac-DEVD)-N′-(ethylthio)carbonyl-Rhodamine 110 (SEQ ID NO.: 23), N-(Ac-DEVD)-N′-(hexylthio)carbonyl-Rhodamine 110 (SEQ ID NO.: 23), N-(Ac-DEVD)-N′-(octylthio)carbonyl-Rhodamine 110 (SEQ ID NO.: 23), N-(Ac-DEVD)-N′-(N-hexyl-N-methylcarbamyl)-Rhodamine 110 (SEQ ID NO.: 23), N-(Ac-DEVD)-N′-(2,3,4,5,6-pentafluorobenzoyl)-Rhodamine (SEQ ID NO.: 23), N-(Ac-DEVD)-N′-(2,3,4,5-tetrafluorobenzoyl)-Rhodamine (SEQ ID NO.: 23) and others disclosed in U.S. Pat. Nos. 6,342,611, 6,335,429 and 6,248,904. Since they are relatively small in size and lipophilic at the same time, many of these substrates can be used in the assays of the invention in the absence of a permeabilization enhancer.

Other useful reporter molecules include Ac-DEVD-pNA (SEQ ID NO.: 23), Ac-DEVD-AMC (SEQ ID NO.: 23), MCA-DEVDAPK(DNP)-OH (SEQ ID NO.: 24), Z-DEVD-AFC (SEQ ID NO.: 23), MCA-VDQMDGW[K-DNP]-NH₂ (SEQ ID NO.: 25), MCA-DEVDAR[K-DNP]-NH₂ (SEQ ID NO.: 26), Z-VDVAD-AFC (SEQ ID NO.: 27), MCA-VDVADGW[K-DNP]-NH₂ (SEQ ID NO.: 28), MCA-VDQVDGW[K-DNP]-NH₂ (SEQ ID NO.: 29), Ac-VEID-pNA (SEQ ID NO.: 30), Ac-VEID-AMC (SEQ ID NO.: 30), Z-VEID-AFC (SEQ ID NO.: 30) and MCA-VQVDGW[K-DNP]-NH₂ (SEQ ID NO.: 31), (CALBIOCHEM, California).

Other fluorogenic reporter molecules useful in the practice of the present invention are disclosed in the following U.S. Pat. Nos.: 4,336,186; 4,557,862; 4,640,893; 5,208,148; 5,227,487; 5,362,628; 5,443,986; 5,556,992; 5,587,490; 5,605,809; 5,698,411; 5,714,342; 5,733,719; 5,776,720, 5,849,513; 5,871,946; 5,897,992; 5,908,750; 5,976,822. Useful reporter molecules are also described in EP 0285179 B1; EP 623599 A1; WO 93/04192; WO 93/10461; WO 96/20721; WO 96/36729; WO 98/57664; Ganesh, S. et al., Cytometry 20:334-340 (1995); Haugland, R. and Johnson, I., J. Fluorescence 3:119-127 (1993); Haugland, R., Biotechnic and Histochemistry 70:243-251 (1995); Haugland, R., Molecular Probes Handbook of Fluorescent Probes and Research Chemicals, pp. 28 and 54, 6th Ed. (1996); Holskin, B., et al., Anal. Biochem. 226:148-155 (1995); Johnson, A., et al., Anal. Chem. 65:2352-2359 (1993); Klingel, S., et al., Methods in Cell Biology 41:449-459 (1994); Leytus, S., et al., Biochem. J. 215:253-260 (1983); Leytus, S., et al., Biochem. J. 209:299-307 (1983); Matayoshi, E., et al., Science 247:954-958 (1990); Morliere, P., et al., Biochem. Biophys. Res. Commun. 146:107-113 (1987); O'Boyle, D., et al., Virology 236:338-347 (1997); Richards, A., et al., J. Biol. Chem. 265:7733-7736 (1990); Rothe, G., et al., Biol. Chem. Hoppe-Seyler 373:547-554 (1992); Stevens, J., et al., Eur. J. Biochem. 226:361-367 (1994); Tamburini, P., et al., Anal. Biochem. 186:363-368 (1990); Thornberry, N., et al., J. Biol. Chem. 272:17907-17911 (1997); Toth, M. and Marshall, G., Int. J. Peptide Protein Res. 36:544-550 (1990); Tyagi, S. and Carter, C., Anal. Biochem. 200:143-148 (1992); Weber, J. “Adenovirus Endopeptidase and Its Role in Virus Infection” in The Molecular Repertoir of Adenoviruses I, Doerfler, W. and Bohm, P. eds., pp. 227-235, Springer Press, New York (1995); Zhang, R., et al., J. Virology 71:6208-6213 (1997); Mangel, W., et al., Biol. Chem. Hoppe-Seyler 373:433-440 (1992); Bonneau, P., et al., Anal. Biochem. 255:59-65 (1998); and

DiIanni, C., et al., J. Biol. Chem. 268:25449-25454 (1993).

As used herein, the abbreviations for any protective groups, amino acids, and other compounds, are, unless indicated otherwise, in accord with their common usage, or recognized abbreviations.

II. Therapeutic Methods

One embodiment of the invention relates to compounds which bind TIPRAIP and induce activation of apoptosis. Another embodiment of the invention relates to pharmaceutical formulations of these compounds, and methods of administration of compositions comprising these compounds for preventing, treating or ameliorating a disease responsive to induction of the caspase cascade in an animal. Another embodiment of the invention pertains to a method of treating, preventing or ameliorating a disease in an animal comprising administering to the animal a composition comprising a compound which binds specifically to an TIPRAIP.

The present invention includes a therapeutic method useful to modulate in vivo apoptosis or in vivo neoplastic disease, comprising administering to a subject in need of such treatment an effective amount of a TIPRAIP binding compound, or a pharmaceutically acceptable salt or prodrug of a TIPRAIP binding compound described herein, which functions as a caspase cascade activator and inducer of apoptosis.

The present invention also includes a therapeutic method comprising administering to an animal an effective amount of a TIPRAIP binding compound, or a pharmaceutically acceptable salt or prodrug of the TIPRAIP binding compound, wherein the therapeutic method is useful to treat cancer, which is a group of diseases characterized by the uncontrolled growth and spread of abnormal cells.

In practicing the therapeutic methods, effective amounts of compositions containing therapeutically effective concentrations of the TIPRAIP binding compounds formulated for oral, intravenous, local and topical application (for the treatment of neoplastic diseases and other diseases in which caspase cascade mediated physiological responses are implicated), are administered to an individual exhibiting the symptoms of one or more of these disorders. The amounts are effective to ameliorate or eliminate one or more symptoms of the disorder. An effective amount of a TIPRAIP binding compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In another embodiment, a pharmaceutical composition comprising a TIPRAIP binding compound, or a pharmaceutically acceptable salt of a TIPRAIP binding compound described herein, which functions as a caspase cascade activator and inducer of apoptosis in combination with a pharmaceutically acceptable vehicle, is provided.

Another embodiment of the present invention is directed to a composition effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known cancer chemotherapeutic agent, or a pharmaceutically acceptable salt of the agent. Examples of known anti-cancer agents which can be used for combination therapy include, but are not limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; and antibodies, such as Herceptin® and Rituxan®. Other known anti-cancer agents, which can be used for combination therapy, include arsenic trioxide, gamcitabine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In practicing the methods of the present invention, the TIPRAIP binding compound of the invention may be administered together with the at least one known chemotherapeutic agent as part of a unitary pharmaceutical composition. Alternatively, the TIPRAIP binding compound of the invention may be administered apart from the at least one known cancer chemotherapeutic agent. In this embodiment, the TIPRAIP binding compound of the invention and the at least one known cancer chemotherapeutic agent are administered substantially simultaneously, i.e. the TIPRAIP binding compounds are administered at the same time or one after the other, so long as the TIPRAIP binding compounds reach therapeutic levels for a period of time in the blood.

It has been reported that alpha-1-adrenoceptor antagonists, such as doxazosin, terazosin, and tamsulosin can inhibit the growth of prostate cancer cell via induction of apoptosis (Kyprianou, N., et al., Cancer Res 60:4550-4555, (2000)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known alpha-1-adrenoceptor antagonists, or a pharmaceutically acceptable salt of the agent. Examples of known alpha-1-adrenoceptor antagonists, which can be used for combination therapy include, but are not limited to, doxazosin, terazosin, and tamsulosin.

It has been reported that sigma-2 receptors are expressed in high densities in a variety of tumor cell types (Vilner, B. J., et al., Cancer Res. 55: 408-413 (1995)) and that sigma-2 receptor agonists, such as CB-64D, CB-184 and haloperidol activate a novel apoptotic pathway and potentiate antineoplastic drugs in breast tumor cell lines. (Kyprianou, N., et al., Cancer Res. 62:313-322 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known sigma-2 receptor agonists, or a pharmaceutically acceptable salt of the agent. Examples of known sigma-2 receptor agonists, which can be used for combination therapy include, but are not limited to, CB-64D, CB-184 and haloperidol.

It has been reported that combination therapy with lovastatin, a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known HMG-CoA reductase inhibitor, or a pharmaceutically acceptable salt of the agent. Examples of known HMG-CoA reductase inhibitors, which can be used for combination therapy include, but are not limited to, lovastatin, simvastatin, pravastatin, fluvastatin, atorvastatin and cerivastatin.

It has been reported that HIV protease inhibitors, such as indinavir or saquinavir, have potent anti-angiogenic activities and promote regression of Kaposi sarcoma (Sgadari, C., et al., Nat. Med. 8:225-232 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known HIV protease inhibitor, or a pharmaceutically acceptable salt of the agent. Examples of known HIV protease inhibitors, which can be used for combination therapy include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

It has been reported that synthetic retinoids, such as fenretinide (N-(4-hydroxyphenyl)retinamide, 4HPR), have good activity in combination with other chemotherapeutic agents, such as cisplatin, etoposide or paclitaxel in small-cell lung cancer cell lines (Kalemkerian, G. P., et al., Cancer Chemother. Pharmacol. 43:145-150 (1999)). 4HPR also was reported to have good activity in combination with gamma-radiation on bladder cancer cell lines (Zou, C., et al., Int. J. Oncol. 13:1037-1041 (1998)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known retinoid and synthetic retinoid, or a pharmaceutically acceptable salt of the agent. Examples of known retinoids and synthetic retinoids, which can be used for combination therapy include, but are not limited to, bexarotene, tretinoin, 13-cis-retinoic acid, 9-cis-retinoic acid, α-difluoromethylomithine, ILX23-7553, fenretinide, and N-4-carboxyphenyl retinamide.

It has been reported that proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known proteasome inhibitor, or a pharmaceutically acceptable salt of the agent. Examples of known proteasome inhibitors, which can be used for combination therapy include, but are not limited to, lactacystin, MG-132, and PS-341.

It has been reported that tyrosine kinase inhibitors, such as STI571 (Imatinib mesilate, Gleevec), have potent synergetic effect in combination with other anti-leukemic agents, such as etoposide (Liu, W. M., et al. Br. J. Cancer 86:1472-1478 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known tyrosine kinase inhibitor, or a pharmaceutically acceptable salt of the agent. Examples of known tyrosine kinase inhibitors, which can be used for combination therapy include, but are not limited to, gleevec, ZD1839 (Iressa), SH268, genistein, CEP2563, SU6668, SU11248, and EMD121974.

It has been reported that prenyl-protein transferase inhibitors, such as farnesyl protein transferase inhibitor R115777, possess preclinical antitumor activity against human breast cancer (Kelland, L. R., et. al., Clin. Cancer Res. 7:3544-3550 (2001)). Synergy of the protein farnesyltransferase inhibitor SCH66336 and cisplatin in human cancer cell lines also has been reported (Adjei, A. A., et al., Clin. Cancer. Res. 7:1438-1445 (2001)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of the agent. Examples of known prenyl-protein transferase inhibitors, which can be used for combination therapy include, but are not limited to, R115777, SCH66336, L-778,123, BAL9611 and TAN-1813.

It has been reported that cyclin-dependent kinase (CDK) inhibitors, such as flavopiridol, have potent synergetic effect in combination with other anticancer agents, such as CPT-11, a DNA topoisomerase I inhibitor in human colon cancer cells (Motwani, M., et al., Clin. Cancer Res. 7:4209-4219, (2001)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known cyclin-dependent kinase inhibitor, or a pharmaceutically acceptable salt of the agent. Examples of known cyclin-dependent kinase inhibitor, which can be used for combination therapy include, but are not limited to, flavopiridol, UCN-01, roscovitine and olomoucine.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16(No. 4 Suppl. 3):17-21 (2002)). Therefore, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with at least one known COX-2 inhibitors, or a pharmaceutically acceptable salt of the agent. Examples of known COX-2 inhibitors, which can be used for combination therapy include, but are not limited to, celecoxib, valecoxib, and rofecoxib.

It has been reported in clinical studies that regular administration of non-steroidal anti-inflammatory drugs (NSAIDs) reduces the risk of breast cancer. See Study: Why aspirin, fiber prevent cancer, posted Wenesday, Apr. 9, 2003 at http://www.cnn.com/2003/Health/04/09/health.cancer. aspirin.reut/index.html. It has also been reported that in colon cancer cells, NSAIDs prevent interleukin-6 from activating STAT1; STAT1 prevents cellular suicide. Id. Hence, NSAIDs are believed to make cells more conducive to apoptosis. Therefore, another embodiment of the present invention is directed to compositions and methods effective to inhibit neoplasia comprising an TIPRAIP binding compound, or a pharmaceutically acceptable salt or prodrug of an TIPRAIP binding compound described herein, which functions as a caspase cascade activator and inducer of apoptosis, in combination with at least one known NSAID, or a pharmaceutically acceptable salt of the agent. Examples of known NSAIDs, which can be used for combination therapy include, but are not limited to, ibuprofen, aspirin and sulindac.

Another embodiment of the present invention is directed to compositions and methods effective to inhibit neoplasia comprising a bioconjugate of a TIPRAIP binding compound described herein, which functions as a caspase cascade activator and inducer of apoptosis, in bioconjugation with at least one known therapeutically useful antibody, such as Herceptin® or Rituxan®, growth factors, such as DGF, NGF; cytokines, such as IL-2, IL-4, or any molecule that binds to the cell surface. The antibodies and other molecules will deliver a TIPRAIP binding compound described herein to its targets and make it an effective anticancer agent. The bioconjugates could also enhance the anticancer effect of therapeutically useful antibodies, such as Herceptin® or Rituxan®.

Similarly, another embodiment of the present invention is directed to compositions and methods effective to inhibit neoplasia comprising a TIPRAIP binding compound, or a pharmaceutically acceptable salt or prodrug of a TIPRAIP binding compound described herein, which functions as a caspase cascade activator and inducer of apoptosis, in combination with radiation therapy. In this embodiment, the TIPRAIP binding compound of the invention may be administered at the same time as the radiation therapy is administered or at a different time.

Yet another embodiment of the present invention is directed to compositions and methods effective for post-surgical treatment of cancer, comprising a TIPRAIP binding compound, or a pharmaceutically acceptable salt or prodrug of a TIPRAIP binding compound described herein, which functions as a caspase cascade activator and inducer of apoptosis. The invention also relates to a method of treating cancer by surgically removing the cancer and then treating the animal with one of the pharmaceutical compositions described herein.

A wide range of immune mechanisms operate rapidly following exposure to an infectious agent. Depending on the type of infection, rapid clonal expansion of the T and B lymphocytes occurs to combat the infection. The elimination of the effector cells following an infection is one of the major mechanisms maintaining immune homeostasis. This deletion of reactive cells has been shown to be regulated by a phenomenon known as apoptosis. Autoimmune diseases have been lately identified as a consequence of deregulated cell death. In certain autoimmune diseases, the immune system directs its powerful cytotoxic effector mechanisms against specialized cells, such as oligodendrocytes in multiple sclerosis, the beta cells of the pancreas in diabetes mellitus, and thyrocytes in Hashimoto's thyroiditis (Ohsako, S., et al., Cell Death Differ. 6(1):13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly and generalized lymphadenopathy, hypergammaglobulinemia, and autoantibody formation. (Infante, A. J., et al., J. Pediatr. 133(5):629-633 (1998) and Vaishnaw, A. K., et al., J. Clin. Invest. 103(3):355-363 (1999)). It was reported that overexpression of Bcl-2, which is a member of the Bcl-2 gene family of programmed cell death regulators with anti-apoptotic activity, in developing B cells of transgenic mice, in the presence of T cell dependent costimulatory signals, results in the generation of a modified B cell repertoire and in the production of pathogenic autoantibodies (Lopez-Hoyos, M., et al., Int. J. Mol. Med. 1(2):475-483 (1998)). It is therefore, evident that many types of autoimmune disease are caused by defects of the apoptotic process and one treatment strategy would be to turn on apoptosis in the lymphocytes that are causing autoimmune disease (O'Reilly, L. A. & Strasser, A., Inflamm. Res. 48(1):5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al., J. Immunol. 162(1):603-608 (1999)) reported that by direct injection of DNA expression vectors encoding FasL into the inflamed thyroid, the development of lymphocytic infiltration of the thyroid was inhibited and induction of the death of infiltrating T cells was observed. These results show that FasL expression on thyrocytes may have a curative effect on ongoing EAT by inducing death of pathogenic autoreactive infiltrating T lymphocytes.

Bisindolylmaleimide VIII is known to potentiate Fas-mediated apoptosis in human astrocytoma 1321N1 cells and in Molt-4T cells, both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. (Zhou, T., et al, Nat. Med. 5(1):42-8 (1999)) reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore, the application of a Fas-dependent apoptosis enhancer, such as bisindolylmaleimide VIII, may be therapeutically useful for the more effective elimination of detrimental cells and inhibition of T cell-mediated autoimmune diseases. Therefore, an effective amount of a TIPRAIP binding compound, or a pharmaceutically acceptable salt or prodrug of a TIPRAIP binding compound described herein, which functions as a caspase cascade activator and inducer of apoptosis, should be an effective treatment for autoimmune disease.

Psoriasis is a chronic skin disease, which is characterized by scaly red patches. Psoralen plus ultraviolet A (PUVA) is a widely used and effective treatment for psoriasis vulgaris and Coven, T. R., et al., Photodermatol. Photoimmunol. Photomed. 15(1):22-7 (1999), reported that lymphocytes treated with psoralen 8-MOP or TMP plus UVA displayed DNA degradation patterns typical of apoptotic cell death. Ozawa, M., et al., J. Exp. Med. 189(4):711-718 (1999) reported that induction of T cell apoptosis could be the main mechanism by which 312-nm UVB resolves psoriasis skin lesions. Low doses of methotrexate may be used to treat psoriasis to restore a clinically normal skin. Heenen, M., et al., Arch. Dermatol. Res. 290(5):240-245 (1998), reported that low doses of methotrexate may induce apoptosis and this mode of action could explain the reduction in epidermal hyperplasia during treatment of psoriasis with methotrexate. Therefore, an effective amount of a TIPRAIP binding compound, or a pharmaceutically acceptable salt or prodrug of a TIPRAIP binding compound described herein, which functions as a caspase cascade activator and inducer of apoptosis, should be an effective treatment for psoriasis.

Synovial cell hyperplasia is a characteristic of patients with rheumatoid arthritis (RA). Excessive proliferation of RA synovial cells that, in addition, are defective in synovial cell death might be responsible for the synovial cell hyperplasia. Wakisaka, S., et al., Clin. Exp. Immunol. 114(1):119-28 (1998), found that, although RA synovial cells could die via apoptosis through Fas/FasL pathway, apoptosis of synovial cells was inhibited by proinflammatory cytokines present within the synovium, and suggested that inhibition of apoptosis by the proinflammatory cytokines may contribute to the outgrowth of synovial cells and lead to pannus formation and the destruction of joints in patients with RA. Therefore, an effective amount of a TIPRAIP binding compound, or a pharmaceutically acceptable salt or prodrug of a TIPRAIP binding compound described herein, which functions as a caspase cascade activator and inducer of apoptosis, should be an effective treatment for rheumatoid arthritis.

There has been an accumulation of convincing evidence that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol. 61(4):375-80 (1997)). Boirivant, M., et al., Gastroenterology 116(3):557-65 (1999), reported that lamina propria T cells isolated from areas of inflammation in Crohn's disease, ulcerative colitis, and other inflammatory states manifest decreased CD2 pathway-induced apoptosis, and that studies of cells from inflamed Crohn's disease tissue, indicate that this defect is accompanied by elevated Bcl-2 levels. Therefore an effective amount of a TIPRAIP binding compound, or a pharmaceutically acceptable salt or prodrug of a TIPRAIP binding compound described herein, which functions as a caspase cascade activator and inducer of apoptosis, should be an effective treatment for inflammation.

Caspase cascade activators and inducers of apoptosis may also be a desirable therapy in the elimination of pathogens, such as HIV, Hepatitis C and other viral pathogens. The long lasting quiecence, followed by disease progression, may be explained by an anti-apoptotic mechanism of these pathogens leading to persistent cellular reservoirs of the virions. It has been reported that HIV-1infected T leukemia cells or peripheral blood mononuclear cells (PBMCs) underwent enhanced viral replication in the presence of the caspase inhibitor Z-VAD-fmk. Furthermore, Z-VAD-fmk also stimulated endogenous virus production in activated PBMCs derived from HIV-1-infected asymptomatic individuals (Chinnaiyan, A., et al., Nat. Med. 3:333 (1997)). Therefore, apoptosis may serve as a beneficial host mechanism to limit the spread of HIV and new therapeutics using caspase/apoptosis activators may be useful to clear viral reservoirs from the infected individuals. Similarly, HCV infection also triggers anti-apoptotic mechanisms to evade the host's immune surveillance leading to viral persistence and hepatocarcinogenesis (Tai, D. I., et al. Hepatology 3:656-64 (2000)). Therefore, apoptosis inducers may be useful as therapeutics for HIV and other infectious disease.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, may be useful as therapeutics for in-stent restenosis.

Compositions within the scope of this invention include all compositions wherein the TIPRAIP binding compounds of the present invention are contained in an amount which is effective to achieve its intended purpose. While individual needs vary, determination of optimal ranges of effective amounts of each component is within the skill of the art. Typically, the TIPRAIP binding compounds may be administered to mammals, e.g. humans, orally at a dose of 0.0025 to 100 mg/kg, or an equivalent amount of the pharmaceutically acceptable salt thereof, per day of the body weight of the mammal being treated for apoptosis-mediated disorders. The TIPRAIP binding compounds may be administered to mammals, e.g. humans, intravenously at a dose of 0.025 to 200 mg/kg, or an equivalent amount of the pharmaceutically acceptable salt thereof, per day of the body weight of the mammal being treated for apoptosis-mediated disorders. Preferably, approximately 0.01 to approximately 50 mg/kg is orally administered to treat or prevent such disorders. For intramuscular injection, the dose is generally approximately one-half of the oral dose. For example, a suitable intramuscular dose would be approximately 0.0025 to approximately 50 mg/kg, and most preferably, from approximately 0.01 to approximately 10 mg/kg. If a known cancer chemotherapeutic agent is also administered, it is administered in an amount which is effective to achieve its intended purpose. The amounts of such known cancer chemotherapeutic agents effective for cancer are well known to those of skill in the art.

The unit oral dose may comprise from approximately 0.01 to approximately 50 mg, preferably approximately 0.1 to approximately 10 mg of the TIPRAIP binding compound of the invention. The unit dose may be administered one or more times daily as one or more tablets, each containing from approximately 0.1 to approximately 10, conveniently approximately 0.25 to 50 mg of the TIPRAIP binding compound or its solvates.

In a topical formulation, the TIPRAIP binding compound may be present at a concentration of approximately 0.01 to 100 mg per gram of carrier.

In addition to administering the TIPRAIP binding compound as a raw chemical, the TIPRAIP binding compounds of the invention may be administered as part of a pharmaceutical preparation containing suitable pharmaceutically acceptable carriers comprising excipients and auxiliaries, which facilitate processing of the TIPRAIP binding compounds into preparations that can be used pharmaceutically. Preferably, the preparations, particularly those preparations, which can be administered orally and which can be used for the preferred type of administration, such as tablets, dragees, and capsules, and also preparations, which can be administered rectally, such as suppositories, as well as suitable solutions for administration by injection or orally, contain from approximately 0.01 to 99 percent, preferably from approximately 0.25 to 75 percent of active TIPRAIP binding compound(s), together with the excipient.

Also included within the scope of the present invention are the non-toxic pharmaceutically acceptable salts of the TIPRAIP binding compounds of the present invention. Acid addition salts are formed by mixing a solution of the particular apoptosis inducer of the present invention with a solution of a pharmaceutically acceptable non-toxic acid, such as hydrochloric acid, hydrobromic acid, fumaric acid, maleic acid, succinic acid, acetic acid, citric acid, lactic acid, tartaric acid, carbonic acid, phosphoric acid, sulfuric acid, oxalic acid, and the like. Basic salts are formed by mixing a solution of the particular apoptosis inducer of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the TIPRAIP binding compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active TIPRAIP binding compounds with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active TIPRAIP binding compound doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active TIPRAIP binding compounds in the form of granules, which may be mixed with fillers, such as lactose, binders such as starches, and/or lubricants such as talc or magnesium stearate and, optionally, stabilizers. In soft capsules, the active TIPRAIP binding compounds are preferably dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active TIPRAIP binding compounds with a suppository base. Suitable suppository bases are, e.g. natural or synthetic triglycerides, or paraffin hydrocarbons. In addition, it is also possible to use gelatin rectal capsules, which consist of a combination of the active TIPRAIP binding compounds with a base. Possible base materials include, e.g. liquid triglycerides, polyethylene glycols, or paraffin hydrocarbons.

Suitable formulations for parenteral administration include aqueous solutions of the active TIPRAIP binding compounds in water-soluble form, e.g. water-soluble salts and alkaline solutions. In addition, suspensions of the active TIPRAIP binding compounds as appropriate oily injection suspensions may be administered. Suitable lipophilic solvents or vehicles include fatty oils, e.g. sesame oil; or synthetic fatty acid esters, e.g. ethyl oleate or triglycerides or polyethylene glycol-400 (the TIPRAIP binding compounds are soluble in PEG-400). Aqueous injection suspensions may contain substances, which increase the viscosity of the suspension include, e.g. sodium carboxymethyl cellulose, sorbitol, and/or dextran. Optionally, the suspension may also contain stabilizers.

In accordance with one aspect of the present invention, TIPRAIP binding compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes approximately 40 parts water, approximately 20 parts beeswax, approximately 40 parts mineral oil, and approximately 1 part almond oil.

Ointments may be formulated by mixing a solution of the active ingredient in a vegetable oil, such as almond oil with warm soft paraffin and allowing the mixture to cool. A typical example of such an ointment is one which includes approximately 30% almond oil and approximately 70% white soft paraffin by weight.

Also included within the scope of the present invention are dosage forms of the TIPRAIP binding compounds, in which the oral pharmaceutical preparations comprise an enteric coating. The term “enteric coating” is used herein to refer to any coating over an oral pharmaceutical dosage form that inhibits dissolution of the active ingredient in acidic media, but dissolves rapidly in neutral to alkaline media and has good stability to long-term storage. Alternatively, the dosage form having an enteric coating may also comprise a water soluble separating layer between the enteric coating and the core.

The core of the enterically coated dosage form comprises a TIPRAIP binding compound. Optionally, the core also comprises pharmaceutical additives and/or excipients. The separating layer may be a water soluble inert TIPRAIP binding compound or polymer for film coating applications. The separating layer is applied over the core by any conventional coating technique known to one of ordinary skill in the art. Examples of separating layers include, but are not limited to sugars, polyethylene glycol, polyvinylpyrrolidone, polyvinyl alcohol, hydroxypropyl cellulose, polyvinyl acetal diethylaminoacetate and hydroxypropyl methylcellulose. The enteric coating is applied over the separating layer by any conventional coating technique. Examples of enteric coatings include, but are not limited to cellulose acetate phthalate, hydroxypropyl methylcellulose phthalate, polyvinyl acetate phthalate, carboxymethylethylcellulose, copolymers of methacrylic acid and methacrylic acid methyl esters, such as Eudragit®L 12,5 or Eudragit®L 100 (R \overline{h}m Pharma), water based dispersions such as Aquateric® (FMC Corporation), Eudragit®L 100-55 (R \overline{h}m Pharma) and Coating CE 5142 (BASF), and those containing water soluble plasticizers such as Citroflex® (Pfizer). The final dosage form is either an enteric coated tablet, capsule or pellet.

III. Polypeptide and Polynucleotide Sequences

This section lists non-limiting examples of TIPRAIPs and the corresponding nucleotides which encode these TIPRAIPs. A sequence listing of these polypeptides and polynucleotides is provided below. These polypeptide and polynucleotide sequences are useful with the screening methods of the present invention.

A. Tail Interacting Protein Related Apoptosis Inducing Proteins (TIPRAIPs)

Non-limiting examples of TIPRAIPs include Cargo selection protein (mannose 6 phosphate receptor binding pr) [Homo sapiens] (SEQ ID NO.:1) (NCBI Accession No. XP_{—}012862); Cargo selection protein (mannose 6 phosphate receptor binding pr) [Homo sapiens] (SEQ ID NO.: 2) (NCBI Accession No. NP_{—}005808); Placental protein 17b1; PP17b1 [Homo sapiens] (SEQ ID NO.: 3) (NCBI Accession No. AAD11622); Placental protein 17a2; PP17a2 [Homo sapiens] (SEQ ID NO.: 4) (NCBI Accession No. AAD11619); Cargo selection protein (mannose 6 phosphate receptor binding protein) [Homo sapiens] (SEQ ID NO.:5) (NCBI Accession No. AAH05818); Cargo selection protein (mannose 6 phosphate receptor binding protein) [Homo sapiens] (SEQ ID NO.: 6) (NCBI Accession No. AAH19278); Cargo selection protein TIP47 [Homo sapiens] (SEQ ID NO.: 7) (NCBI Accession No. AAC39751); Cargo selection protein (mannose 6 phosphate receptor binding protein) [Homo sapiens] (SEQ ID NO.: 8) (NCBI Accession No. AAH07566); Cargo selection protein (mannose 6 phosphate receptor binding protein) [Homo sapiens] (SEQ ID NO.: 9) (NCBI Accession No. AAH01590); Placental protein 17a1; PP17a1 [Homo sapiens] (SEQ ID NO.: 10) (NCBI Accession No. AAD11620); Cargo selection protein TIP47 (47 kDa mannose 6-phosphate receptor-binding protein) (47 kDa MPR-binding protein) (Placental protein 17) [Homo sapiens] (SEQ ID NO.: 11) (NCBI Accession No. O60664); and Sequence 1 from U.S. Pat. No. 5,989,820 [Unknown] (SEQ ID NO.: 12) (NCBI Accession No. AAE37397).

B. Nucleotide Sequences Encoding for Tail Interacting Protein Related Apoptosis Inducing Proteins (TIPRAIPs)

Non-limiting examples of nucleotide sequences which encode for TIPRAIPs include Homo sapiens cargo selection protein (mannose 6 phosphate receptor binding protein) (TIP47), mRNA [Homo sapiens] (SEQ ID NO.: 13) (NCBI Accession No. XM_{—}012862): Homo sapiens cargo selection protein (mannose 6 phosphate receptor binding protein) (TIP47), mRNA [Homo sapiens] (SEQ ID NO.: 14) (NCBI Accession No. NM_{—}005817); Homo sapiens placental protein 17b1 (PP17) mRNA, complete cds [Homo sapiens] (SEQ ID NO.: 15) (NCBI Accession No. AF055574); Homo sapiens placental protein 17a2 (PP 17) mRNA, complete cds [Homo sapiens] (SEQ ID NO.: 16) (NCBI Accession No. AF051314); Homo sapiens, cargo selection protein (mannose 6 phosphate receptor binding protein), clone MGC:11117 IMAGE:3833411, mRNA, complete cds [Homo sapiens] (SEQ ID NO.: 17) (NCBI Accession No. BC005818); Homo sapiens, cargo selection protein (mannose 6 phosphate receptor binding protein), clone MGC:3816 IMAGE:2905275, mRNA, complete cds [Homo sapiens] (SEQ ID NO.: 18) (NCBI Accession No. BC019278); Homo sapiens cargo selection protein TIP47 (TIP47) mRNA, complete cds [Homo sapiens] (SEQ ID NO.: 19) (NCBI Accession No. AF057140); Homo sapiens, cargo selection protein (mannose 6 phosphate receptor binding protein), clone MGC:15516 IMAGE:3028104, mRNA, complete cds [Homo sapiens] (SEQ ID NO.: 20) (NCBI Accession No. BC007566); Homo sapiens, cargo selection protein (mannose 6 phosphate receptor binding protein), clone MGC:2012 IMAGE:2987965, mRNA, complete cds [Homo sapiens] (SEQ ID NO.: 21) (NCBI Accession No. BC001590); and Homo sapiens placental protein 17a1 (PP17) mRNA, complete cds [Homo sapiens] (SEQ ID NO.: 22) (NCBI Accession No. AF051315).

The skilled artisan recognizes the presence of human and statistical error in sequencing nucleotides. Nucleotide sequences determined by automation are typically at least about 90% identical, more typically at least about 95% to at least about 99.9% identical to the actual nucleotide sequence of the sequenced nucleotide molecule. The actual sequence can be more precisely determined by other approaches including manual nucleotide sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The skilled artisan also recognizes that nucleotides encoding TIPRAIPs may include splice variants of the nucleotides described herein.

IV. Expression Vectors and Transfected Cells

The present invention also relates to vectors which include the isolated nucleotide molecules of the present invention, host cells which are genetically engineered with the recombinant vectors, and the production of TIPRAIP by recombinant techniques. TIPRAIP may be extracted from cultures of the below described transfected cells and used for the homogenous and heterogenous assays described herein. Alternatively, TIPRAIP can be synthesized for these assays using peptide synthetic techniques known in the art. Also, the below described expression vectors and transfected cells are useful for whole cell assays described herein.

The polynucleotides may be joined to a vector containing a selectable marker for propagation in a host. Generally, a plasmid vector is introduced in a precipitate, such as a calcium phosphate precipitate, or in a complex with a charged cationic lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs may include a translation initiating at the beginning and a termination codon (UAA, UGA or UAG) appropriately positioned at the end of the polypeptide to be translated.

As indicated, the expression vectors may include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Vectors which may be used in bacteria include pQE70, pQE60 and pQE-9, available from Qiagen; pBS vectors, Phagescript vectors, Bluescript vectors, pNH8A, pNH 16a, pNH 18A, pNH46A, available from Stratagene; and ptrc99a, pKK223-3, pKK233-3, pDR540, pRIT5 available from Pharmacia. Eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of nucleotides into the host cell can be affected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986). Methods of formulating nucleotides with compositions (e.g., lipids) to facilitate introduction of the nucleotide into the cell are disclosed in, for example, U.S. Pat. Nos. 4,897,355, 4,394,448, 4,235,871, 4,231,877, 4,224,179, 4,753,788, 4,673,567, 4,247,411, 4,814,270, 5,279,833, and 5,753,613; and in published U.S. patent application Ser. No. 2002/0086849. Other methods for transfecting cells which are useful for the present invention include those described in U.S. Pat. Nos. 5,547,932; 5,981,273; 6,022,735; 6,077,663; 6,274,322; and Published International Application No. WO 00/43494.

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. An example of a fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof.

TIPRAIP can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, or hydroxylapatite chromatography. High performance liquid chromatography (“HPLC”) can also be employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

V. Homogenous and Heterogenous Screening Assays

One aspect of the present invention relates to a method of identifying TIPRAIP binding compounds using homogenous or heterogenous binding assays. This may be accomplished by using non-competitive binding assays, or assays in which test compounds compete with 3-(4-azidophenyl)-5-(3-chloro-thiophen-2-yl)-[1,2,4]-oxadiazole or a substituted 3-aryl-5-aryl-[1,2,4]-oxadiazole such as those described herein or in nonprovisional U.S. patent application No. 10/164,705, filed Jun. 10, 2002 (Cai et al.); or in provisional U.S. Patent Application Ser. No. 60/433,953, filed Dec. 18, 2002 (Cai et al.), or the compounds and compositions described in the Examples below. Any method known to one of ordinary skill in the art that detects binding between a test compound and a protein or antibody may be used in the present invention. These assays may be radioassays, fluorescence polarization assays or other fluorescence techniques, or biotin-avidin based assays. Test compounds capable of binding to TIPRAIP are candidates for activators of apoptosis. Test compounds may be capable of binding to TIPRAIP as strongly or more strongly than 3-(4-azidophenyl)-5-(3-chloro-thiophen-2-yl)-[1,2,4]-oxadiazole or a substituted 3-aryl-5-aryl-[1,2,4]-oxadiazole.

Another aspect of the present invention relates to a method of identifying TIPRAIP binding compounds using antibodies to 3-(4-azidophenyl)-5-(3-chloro-thiophen-2-yl)-[1,2,4]-oxadiazole or a substituted 3-aryl-5-aryl-[1,2,4]-oxadiazole. Such a method relates to detecting binding between i) an antibody to 3-(4-azidophenyl)-5-(3-chloro-thiophen-2-yl)-[1,2,4]-oxadiazole or a substituted 3-aryl-5-aryl-[1,2,4]-oxadiazole and ii) a test compound. Because 3-(4-azidophenyl)-5-(3-chloro-thiophen-2-yl)-[1,2,4]-oxadiazole or substituted 3-aryl-5-aryl-[1,2,4]-oxadiazoles bind TIPRAIP, an antibody which is specific for 3-(4-azidophenyl)-5-(3-chloro-thiophen-2-yl)-[1,2,4]-oxadiazole or a substituted 3-aryl-5-aryl-[1,2,4]-oxadiazole is likely to be specific for other compositions having the physical characteristics that afford TIPRAIP specific binding. Hence, antibodies can be used to screen chemical libraries for other compositions that bind TIPRAIPs and that activate apoptosis. In such assays, the antibody may give rise to a detectable signal upon binding a test compound. For example, the antibodies may be labeled with a fluorophore. Antibodies bound to a test compound may also be detected using radiolabels.

Assays for use in the present invention are preferably high throughput screening methods, capable of screening large numbers of compounds in a rapid fashion. This includes, for example, screening methods that use microbeads or plates having multiple wells.

A. Competitive and Non-Competitive Homogenous Binding Assays

Any homogeneous assay well known in the art can be used in the present invention to determine binding between test compounds of interest and TIPRAIP. For example, radioassays, fluorescence polarization assays and time-resolved fluorescence assays may all be used. Where TIPRAIP is labeled, the assay may be a non-competitive binding assay in which the ability of test compounds to bind TIPRAIP is determined. Where 3-(4-azidophenyl)-5-(3-chloro-thiophen-2-yl)-[1,2,4]-oxadiazole or substituted 3-aryl-5-aryl-[1,2,4]-oxadiazoles are labeled, such as those described in Example 1-3 of this application, the assay may be a competitive binding assay where the ability of a test compound to displace TIPRAIP-bound 3-(4-azidophenyl)-5-(3-chloro-thiophen-2-yl)-[1,2,4]-oxadiazole or substituted 3-aryl-5-aryl-[1,2,4]-oxadiazole is determined.

A homogeneous binding assay used in the present invention, and which uses fluorescence to detect the test compound/TIPRAIP binding, may employ fluorescently labeled 3-(4-azidophenyl)-5-(3-chloro-thiophen-2-yl)-[1,2,4]-oxadiazole or substituted 3-aryl-5-aryl-[1,2,4]-oxadiazoles, or fluorescently labeled TIPRAIP. Any method known to one of ordinary skill in the art can be used to link the fluorophore to 3-(4-azidophenyl)-5-(3-chloro-thiophen-2-yl)-[1,2,4]-oxadiazole or a substituted 3-aryl-5-aryl-[1,2,4]-oxadiazole or polypeptide of interest. See, e.g., Richard P. Haugland, Molecular Probes: Handbook of Fluorescent Probes and Research Chemicals 1992-1994 (5th edit, 1994, Molecular Probes, Inc.).

Fluorescence Polarization (FP), first described by Perrin, J. Phys. Rad. 1:390-401 (1926), is based upon the finding that the emission of light by a fluorophore can be depolarized by a number of factors, the most predominant being rotational diffusion, or, in other words, the rate at which a molecule tumbles in solution. “Polarization” is the measurement of the average angular displacement of the fluorophore that occurs between the absorption and subsequent emission of a photon. This angular displacement of the fluorophore is, in turn dependent upon the rate and extent of rotational diffusion during the lifetime of the excited state, which is influenced by the viscosity of the solution and the size and shape of the diffusing fluorescent species. If viscosity and temperature are held constant, the polarization is directly related to the molecular volume or size of the fluorophore. In addition, the polarization value is a dimensionless number (being a ratio of vertical and horizontal fluorescent intensities) and is not affected by the intensity of the fluorophore.

In fluorescent assays, light from a monochromatic source passes through a vertical polarizing filter to excite fluorescent molecules in a sample tube. Only those molecules that are orientated in the vertically polarized plane absorb light, become excited, and subsequently emit light. The emission light intensity is measured both parallel and perpendicular to the exciting light. The fraction of the original incident, vertical light intensity that is emitted in the horizontal plane is a measure of the amount of rotation that the fluorescently labeled TIPRAIP has undergone during the excited state, and therefore is a measure of its relative size. See, “Introduction to Fluorescence Polarization,” Pan Vera Corp., Madison, Wis., Jun. 17, 1996. Other publications describing the fluorescence polarization technique include G. Weber, Adv. Protein Chem. 8:415-459 (1953); W. B. Dandilker, et al., Immunochemistry 10:219-227 (1973); and M. E. Jolley, J. Anal. Toxicol. 5:236-240 (1981); “Chapter 4-Introduction to Fluorescence Polarization, “the FPM-1™ Operators Manual, pp. 9-10, Jolley Consulting and Research, Inc. Grayslake, Ill.; Lynch, B. A., et al., Anal. Biochem. 247:77-82 (1997); Wei, A. P. and Herron, J. N., Anal. Chem. 65:3372-3377 (1993); and Kauvar, L. M, et al., Chem. Biol. 2:107-118 (1995).

The apparatus used in fluorescence polarization techniques are well known in the art. Examples of an apparatus used in fluorescence polarization are given in U.S. Pat. No. 6,482,601 B1; U.S. Pat. No. 6,455,861; U.S. Pat. No. 5,943,129; U.S. Pat. No. 4,699,512 and U.S. Pat. No. 4,548,499. Other specific examples of instruments for use in the invention include, but are limited to, the Sentry-FP® fluorescence polarization instrument (Diachemix Corp., Milwaukee, Wis.); the BEACON® 2000 fluorescence polarization instrument (PanVera, Madison, Wis.); the POLARSCAN® portable fluorescence polarization system (Associates of Cape Cod, Inc., Falmouth, Mass.); the VICTOR® series instruments (PerkinElmer, Inc., Wellesley, Mass.); and the AFFINTY® and SYMMETRY® fluorescence systems (CRi, Inc., Woborn, Mass.).

One embodiment of the invention relates to a non-competitive fluorescent assay. Such an assay employs TIPRAIP covalently attached to a fluorophore. Free TIPRAIP has higher fluorescence intensity than TIPRAIP bound to a test compound. Confer Hwang, et al., Biochemistry 31:11536-11545 (1992). Once the test compound/TIPRAIP complex is formed, it rotates and tumbles more slowly and has less fluorescence intensity. Confer “Introduction to Fluorescence Polarization,” Pan Vera Corp., Madison, Wis., Jun. 17, 1996; Perrin, J. Phys. Rad. 1:390-401 (1926). Hence, when the test compound and TIPRAIP bind, the fluorescence intensity of the labeled TIPRAIP decreases proportional to binding.

In this embodiment, a solution of the labeled TIPRAIP is prepared and its fluorescence polarization is measured. TIPRAIP and the test compound are mixed together and the solution is allowed to reach equilibrium over some time period. The fluorescence of any test compound/TIPRAIP complex which forms is then measured. The decrease in fluorescence intensity is proportional to binding. The test compound binding may be compared to a baseline fluorescence intensity value determined for 3-(4-azidophenyl)-5-(3-chloro-thiophen-2-yl)-[1,2,4]-oxadiazole or a substituted 3-aryl-5-aryl-[1,2,4]-oxadiazole bound to TIPRAIP. Test compounds that bind to TIPRAIP are considered candidates for activators of apoptosis. The skilled artisan will recognize that a variety of parameters such as temperature, time, concentration and pH can be varied to study the binding between the test compound and TIPRAIP.

The baseline fluorescence polarization value is determined by preparing labeled TIPRAIP and measuring its fluorescence polarization. 3-(4-Azidophenyl)-5-(3-chloro-thiophen-2-yl)-[1,2,4]-oxadiazole or a substituted 3-aryl-5-aryl-[1,2,4]-oxadiazole is mixed with labeled TIPRAIP and allowed to equilibrate for a sufficient time to form a complex between the 3-(4-azidophenyl)-5-(3-chloro-thiophen-2-yl)-1,2,4]-oxadiazole or the substituted 3-aryl-5-aryl-[1,2,4]-oxadiazole and TIPRAIP. The fluorescence polarization of the solution comprising the complex is measured. The relative change in the fluorescence polarization is the baseline value against which all other test compounds will be measured. A variety of parameters such as temperature, time, concentration and pH can be varied to develop a range of values for the change in fluorescence polarization under a variety of conditions.

In determining whether a test compound binds to TIPRAIP strongly enough to be considered a candidate for inducing apoptosis, the change in fluorescence polarization between unbound and bound test compound is compared with the change in fluorescence polarization between unbound and bound 3-(4-azidophenyl)-5-(3-chloro-thiophen-2-yl)-[1,2,4]-oxadiazole or substituted 3-aryl-5-aryl-[1,2,4]-oxadiazole. Test compounds that bind as strongly as or more strongly than 3-(4-azidophenyl)-5-(3-chloro-thiophen-2-yl)-[1,2,4]-oxadiazole or substituted 3-aryl-5-aryl-[1,2,4]-oxadiazoles are candidates for activators of apoptosis.

Competitive homogenous fluorescence assays can also be used in the present invention to find new candidates for activating apoptosis. Competitive assays are well known in the art and any method can be used in the present invention. For example, U.S. Pat. No. 6,511,815 B1 describes an assay for quantitating competitive binding of test compounds to proteins utilizing fluorescence polarization.

In this embodiment of the invention, 3-(4-azidophenyl)-5-(3-chloro-thiophen-2-yl)-[1,2,4]-oxadiazole or a substituted 3-aryl-5-aryl-[1,2,4]-oxadiazole is first labeled with a fluorophore. The labeled 3-(4-azidophenyl)-5-(3-chloro-thiophen-2-yl)-[1,2,4]-oxadiazole or substituted 3-aryl-5-aryl-[1,2,4]-oxadiazole is mixed with TIPRAIP in a buffered solution. The mixture is allowed to equilibrate and the fluorescence polarization of the 3-(4-azidophenyl)-5-(3-chloro-thiophen-2-yl)-[1,2,4]-oxadiazole/TIPRAIP (or substituted 3-aryl-5-aryl-[1,2,4]-oxadiazole/TIPRAIP) complex is measured. The test compound is then introduced into the mixture and allowed to equilibrate. Where a given test compound effectively competes for an TIPRAIP binding site, the labeled 3-(4-azidophenyl)-5-(3-chloro-thiophen-2-yl)-[1,2,4]-oxadiazole or labeled substituted 3-aryl-5-aryl-[1,2,4]-oxadiazole will be displaced and become free, labeled 3-(4-azidophenyl)-5-(3-chloro-thiophen-2-yl)-[1,2,4]-oxadiazole or substituted 3-aryl-5-aryl-[1,2,4]-oxadiazole. Because the fluorophore (covalently attached to the 3-(4-azidophenyl)-5-(3-chloro-thiophen-2-yl)-[1,2,4]-oxadiazole or substituted 3-aryl-5-aryl-[1,2,4]-oxadiazole) is no longer associated with the bulky TIPRAIP, it gives rise to a more intense fluorescence polarization signal. Accordingly, in this embodiment, increases in fluorescent signals is proportional to the ability of a test compound to bind TIPRAIP.

In the above assays, several components of the mixture can affect the fluorescence intensity other than the labeled moiety. The polarity of the solvent and non-specific binding molecules can have significant affects on the intensity, which can be incorrectly interpreted. Therefore, an alternative assay for determining test compound/TIPRAIP binding for use in the present invention relies on time-resolved fluorescence techniques, which minimizes the above problems. The method of time-resolved fluorescence is described in detail in I. Hemmilä, et al., “High Throughput Screening. The Discovery of Bioactive Substances,” Chapter 20, J. P. Devlin, ed., Marcel Dekker, Inc., New York (1997). The excited state lifetime of the test compound/TIPRAIP complex is longer than that for the impurities and other components that add background fluorescence. Therefore, the solution comprising the test compound/TIPRAIP complex mixture may be illuminated and after a short period of time on the order of nano to micro seconds, the solution fluorescence is measured.

In one embodiment of a time-resolved competitive fluorescence based homogeneous assay for use in the present invention, the fluorescent signal is generated when TIPRAIP and 3-(4-azidophenyl)-5-(3-chloro-thiophen-2-yl)-[1,2,4]-oxadiazole or a substituted 3-aryl-5-aryl-[1,2,4]-oxadiazole bind. In this embodiment, either TIPRAIP or 3-(4-azidophenyl)-5-(3-chloro-thiophen-2-yl)-[1,2,4]-oxadiazole or a substituted 3-aryl-5-aryl-[1,2,4]-oxadiazole is covalently bound to an energy donating Eu-cryptate having a long-lived fluorescent excited state. The other is attached to an energy-accepting protein, allophycocyanin, having a short fluorescent excited state. Energy transfer occurs between the Eu-cryptate and the allphycocyanin when they are less than 7 nm apart. During the assay, the Eu-cryptate is excited by a pulsed laser, and its fluorescent emission continually re-excites the allphycocyanin, whose fluorescence is measured by a time resolved fluorescence reader. Confer A. J. Kolb, et al., “High Throughput Screening. The Discovery of Bioactive Substances,” Chapter 19, J. P. Devlin, ed., Marcel Dekker, Inc., New York (1997).

In this embodiment of a time-resolved competitive fluorescence based homogeneous assay, the TIPRAIP and 3-(4-azidophenyl)-5-(3-chloro-thiophen-2-yl)-[1,2,4]-oxadiazole (or substituted 3-aryl-5-aryl-[1,2,4]-oxadiazole) attached to the Eu-cryptate or allphycocyanin are mixed together and allowed to equilibrate. Once equilibrated, the fluorescence intensity is measured. The test compound is then introduced into the mixture and allowed to equilibrate. Where a given test compound effectively competes for an TIPRAIP binding site, the labeled 3-(4-azidophenyl)-5-(3-chloro-thiophen-2-yl)-[1,2,4]-oxadiazole or substituted 3-aryl-5-aryl-[1,2,4]-oxadiazole will be displaced and the Eu-cryptate and allphycocyanin will no longer be less than 7 nm apart. Accordingly, the fluorescence intensity will decrease. Hence, in this embodiment, decreases in fluorescent signals is proportional to the ability of a test compound to bind TIPRAIP.

Alternative homogeneous assays for use in the invention include those described in U.S. Pat. No. 6,492,128 B1; U.S. Pat. No. 6,406,913 B1; U.S. Pat. No. 6,326,459 B1; U.S. Pat. No. 5,928,862; U.S. Pat. No. 5,876,946; U.S. Pat. No. 5,612,221; and U.S. Pat. No. 5,556,758.

The skilled artisan will recognize that radiolabels can also be used in homogenous competitive binding assays. In such assays, 3-(4-azidophenyl)-5-(3-chloro-thiophen-2-yl)-[1,2,4]-oxadiazole or a substituted 3-aryl-5-aryl-[1,2,4]-oxadiazole is radiolabeled and allowed to equilibrate with TIPRAIP in solution. Then, a test compound is introduced into the solution and allowed to equilibrate. TIPRAIP (bound either to radiolabeled 3-(4-azidophenyl)-5-(3-chloro-thiophen-2-yl)-[1,2,4]-oxadiazole or a radiolabeled substituted 3-aryl-5-aryl-[1,2,4]-oxadiazole or to the test compound) is then separated from unbound 3-(4-azidophenyl)-5-(3-chloro-thiophen-2-yl)-[1,2,4]-oxadiazole (or substituted 3-aryl-5-aryl-[1,2,4]-oxadiazole) and unbound test compound. Where a test compound is a poor TIPRAIP binder, most of the TIPRAIP will be bound to radiolabeled 3-(4-azidophenyl)-5-(3-chloro-thiophen-2-yl)-[1,2,4]-oxadiazole (or substituted 3-aryl-5-aryl-[1,2,4]-oxadiazole) and this can be detected by a scintillation counter, photoradiography, or other techniques well known in the art. If, however, the test compound is a strong TIPRAIP binder and displaces radiolabeled 3-(4-azidophenyl)-5-(3-chloro-thiophen-2-yl)-[1,2,4]-oxadiazole (or substituted 3-aryl-5-aryl-[1,2,4]-oxadiazole), then most of the TIPRAIP will not be bound to radiolabeled 3-(4-azidophenyl)-5-(3-chloro-thiophen-2-yl)-[1,2,4]-oxadiazole (or substituted 3-aryl-5-aryl-[1,2,4]-oxadiazole). Hence, ability of a test compound to bind TIPRAIP is inversely proportional to the amount of radiolabel detected with the TIPRAIP.

B. Competitive Heterogenous Binding Assays

Detection of the test compound binding to TIPRAIP may also be accomplished using heterogeneous assays. Heterogeneous assays for use in the present invention may be based on radioassays, fluorescence-based assays and biotin-avidin based assays. In heterogenous assays, a first component is attached to a solid phase such as a bead or other solid substrate and one or more additional components are in solution. For example, TIPRAIP may be bound to a bead or other solid substrate and labeled 3-(4-azidophenyl)-5-(3-chloro-thiophen-2-yl)-[1,2,4]-oxadiazole (or substituted 3-aryl-5-aryl-[1,2,4]-oxadiazole) is introduced as a solution. The label may be a radiolabel, chemiluminescent label, fluorescent label, chromogenic label, or other label well known in the art. After the mixture equilibrates and the 3-(4-azidophenyl)-5-(3-chloro-thiophen-2-yl)-[1,2,4]-oxadiazole)/TIPRAIP (or substituted 3-aryl-5-aryl-[1,2,4]-oxadiazole/TIPRAIP) complexes form, a solution of test compound is introduced and allowed to equilibrate to form test compound/TIPRAIP complexes. The beads or solid components are separated from the solutions. This can be done, for example, using magnetic fields where the beads are magnetic. Alternatively, where TIPRAIP is bound to a solid substrate, separation can occur simply by rinsing the solid substrate with water or a buffer to remove any solution containing unbound labeled 3-(4-azidophenyl)-5-(3-chloro-thiophen-2-yl)-[1,2,4]-oxadiazole (or substituted 3-aryl-5-aryl-[1,2,4]-oxadiazole) or unbound test compound. The extent to which TIPRAIP remains associated with the detectably labeled 3-(4-azidophenyl)-5-(3-chloro-thiophen-2-yl)-[1,2,4]-oxadiazole (or substituted 3-aryl-5-aryl-[1,2,4]-oxadiazole) is measured. Such measurements can be performed while TIPRAIP remains bound to the bead or solid substrate. Alternatively, such measurements can be made after TIPRAIP has been removed from the bead or solid substrate. In such competitive binding assays, decreases in signal associated with the detectable label are proportionally related to increases in the ability of test compounds to bind TIPRAIP by displacing 3-(4-azidophenyl)-5-(3-chloro-thiophen-2-yl)-[1,2,4]-oxadiazole (or substituted 3-aryl-5-aryl-[1,2,4]-oxadiazole).

The skilled artisan recognizes that the 3-(4-azidophenyl)-5-(3-chloro-thiophen-2-yl)-[1,2,4]-oxadiazole (or substituted 3-aryl-5-aryl-[1,2,4]-oxadiazole) may also be the component bound to the beads or solid substrate. In such assays, labeled TIPRAIP is introduced as a solution and allowed to equilibrate forming the 3-(4-azidophenyl)-5-(3-chloro-thiophen-2-yl)-[1,2,4]-oxadiazole/TIPRAIP (or substituted 3-aryl-5-aryl-[1,2,4]-oxadiazole/TIPRAIP) complexes. The label may be a radiolabel, chemiluminescent label, fluorescent label, chromogenic label, or other label well known in the art. Then, a test compound is added as a solution. If a test compound displaces 3-(4-azidophenyl)-5 -(3-chloro-thiophen-2-yl)-[1,2,4]-oxadiazole (or substituted 3-aryl-5-aryl-[1,2,4]-oxadiazole), then the TIPRAIP will fall back into solution and not be bound to the bead or solid substrate through 3-(4-azidophenyl)-5-(3-chloro-thiophen-2-yl-[1,2,4]-oxadiazole (or substituted 3-aryl-5-aryl-[1,2,4]-oxadiazole). As described above, the beads or solid substrate are removed from the solution but the solution is retained to measure the extent of the detectable label. Here, increases in signal associated with the detectable label are proportional to the ability of a test compound to bind TIPRAIP.

Solid phase supports for use in the present invention include any insoluble support known in the art that is capable of binding TIPRAIP or 3-(4-azidophenyl)-5-(3-chloro-thiophen-2-yl)-[1,2,4]-oxadiazole or substituted 3-aryl-5-aryl-[1,2,4]-oxadiazoles. This includes, for example, glass and natural and synthetic polymers such as agaroses, polystyrene, polypropylene, polyethylene, dextran, nylon, amylases, natural and modified celluloses, polyacrylamides, and magnetite. The support material may have virtually any possible structural configuration so long as the support-bound molecule is capable of binding to a test compound, 3-(4-azidophenyl)-5-(3-chloro-thiophen-2-yl)-[1,2,4]-oxadiazole (or substituted 3-aryl-5-aryl-[1,2,4]-oxadiazole) or TIPRAIP. Thus, the support configuration may be spherical, as in a bead, or cylindrical, as in the inside surface of a test tube, or the external surface of a rod, or hemishperical surface such as the well of a microtitre plate. Alternatively, the surface may be flat such as a sheet, test strip, etc. Those skilled in the art will note many other suitable carriers for binding 3-(4-azidophenyl)-5-(3-chloro-thiophen-2-yl)-[1,2,4]-oxadiazole or substituted 3-aryl-5-aryl-[1,2,4]-oxadiazoles or TIPRAIP, or will be able to ascertain the same by use of routine experimentation.

An example of a heterogeneous assay for use in the present invention is the radioassay. A good description of a radioassay may be found in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T. Examples of other competitive radioassays are given in U.S. Pat. Nos. 3,937,799; 4,102,455; 4,333,918 and 6,071,705. Inherent in such assays is the need to separate the bead or substrate bound component from the solution component. Various ways of accomplishing the required separation have been developed, including those exemplified in U.S. Pat. Nos. 3,505,019; 3,555,143; 3,646,346; 3,720,760; and 3,793,445. The skilled artisan will recognize that separation can include filtering, centrifuging, washing, or draining the solid substrate to insure efficient separation of the substrate bound and solution phases.

The radioactive isotope or radiolabel can be detected by such means as the use of a gamma counter or a scintillation counter or by audioradiography. Isotopes which are particularly useful for the purpose of the present invention are: ³H. ¹²³I, ¹²⁵I. ¹³¹I. ³⁵S, ³¹P. ¹⁴C, ¹¹¹In, ⁹⁷Ru ⁶⁷Cu, ⁶⁷Ga, ⁶⁸Ga, ⁷²As, ⁸⁹Zr and ²⁰¹Tl. Those of ordinary skill in the art will know of other suitable labels, which may be employed in accordance with the present invention. The binding of these labels TIPRAIP, 3-(4-azidophenyl)-5-(3-chloro-thiophen-2-yl)-[1,2,4]-oxadiazole (or substituted 3-aryl-5-aryl-[1,2,4]-oxadiazole) can be accomplished using standard techniques commonly known to those of ordinary skill in the art. Typical techniques are described by Kennedy, J. H., et al. (Clin. Chim. Acta 70:1-31 (1976)), and Schurs, A. H. W. M., et al. (Clin. Chim. Acta 81:1-40 (1977)). In a particular embodiment, one or more hydrogen and/or carbon atoms of TIPRAIP, 3-(4-azidophenyl)-5-(3-chloro-thiophen-2-yl)-[1,2,4]-oxadiazole or substituted 3-aryl-5-aryl-[1,2,4]-oxadiazole are replaced by ³H and ¹⁴C, by methods well known in the art.

In one embodiment of the invention, TIPRAIP is attached to a solid support. Radiolabeled 3-(4-azidophenyl)-5-(3-chloro-thiophen-2-yl)-[1,2,4]-oxadiazole or substituted 3-aryl-5-aryl-[1,2,4]-oxadiazole is prepared. The bound TIPRAIP is admixed with the solution comprising radiolabeled 3-(4-azidophenyl)-5-(3-chloro-thiophen-2-yl)-[1,2,4]-oxadiazole or substituted 3-aryl-5-aryl-[1,2,4]-oxadiazole. The mixture is allowed to equilibrate for a time period. A test compound is added to the mixture and allowed to equilibrate for some time period. The test compound competes for the binding site of TIPRAIP with the radiolabeled 3-(4-azidophenyl)-5-(3-chloro-thiophen-2-yl)-[1,2,4]-oxadiazole or substituted 3-aryl-5-aryl-[1,2,4]-oxadiazole. The solid support that has bound TIPRAIP is removed from the mixture. The amount of radiolabel associated with TIPRAIP is measured. Decreases in the amount of radiolabel are proportional to the ability of a test compound to displace 3-(4-azidophenyl)-5-(3-chloro-thiophen-2-yl)-[1,2,4]-oxadiazole (or a substituted 3-aryl-5-aryl-[1,2,4]-oxadiazole) and bind TIPRAIP. Alternatively, the radiation of the solution comprising unbound and uncomplexed radiolabeled 3-(4-azidophenyl)-5-(3-chloro-thiophen-2-yl)-[1,2,4]-oxadiazole or substituted 3-aryl-5-aryl-[1,2,4]-oxadiazole can be measured. Using this assay, test compounds that bind to TIPRAIP receptor as strongly or more strongly than 3-(4-azidophenyl)-5-(3-chloro-thiophen-2-yl)-[1,2,4]-oxadiazole or substituted 3-aryl-5-aryl-[1,2,4]-oxadiazole can easily be discovered.

Alternative labels for use in the heterogeneous assays of the present invention include chemiluminescent labels, such as those described in U.S. Pat. No. 4,380,580; and enzyme substrate labels, such as those assays described in U.S. Pat. No. 4,492,751. For example, a fluorescent label may be used.

In these competitive fluorescence-based heterogeneous assays, a solution of fluorescently labeled 3-(4-azidophenyl)-5-(3-chloro-thiophen-2-yl)-[1,2,4]-oxadiazole or substituted 3-aryl-5-aryl-[1,2,4]-oxadiazole is prepared. TIPRAIP is attached to a solid support. The bound TIPRAIP is admixed with the solution comprising fluorescently labeled 3-(4-azidophenyl)-5-(3-chloro-thiophen-2-yl)-[1,2,4]-oxadiazole or substituted 3-aryl-5-aryl-[1,2,4]-oxadiazole. The mixture is allowed to equilibrate for a time period. A test compound is added to the mixture and the mixture is allowed to equilibrate for some time period. The test compound competes for the binding receptor of TIPRAIP with fluorescently labeled 3-(4-azidophenyl)-5-(3-chloro-thiophen-2-yl)-[1,2,4]-oxadiazole or substituted 3-aryl-5-aryl-[1,2,4]-oxadiazole. The solid support that has bound TIPRAIP is removed from the mixture. The amount of fluorescence associated with TIPRAIP attributed to the fluorescent label is measured. Decreases in the amount of this fluorescence are proportional to the ability of a test compound to displace 3-(4-azidophenyl)-5-(3-chloro-thiophen-2-yl)-[1,2,4]-oxadiazole (or a substituted 3-aryl-5-aryl-[1,2,4]-oxadiazole) and bind TIPRAIP. Alternatively, the fluorescence of the solution comprising unbound and uncomplexed fluorescently labeled 3-(4-azidophenyl)-5-(3-chloro-thiophen-2-yl)-[1,2,4]-oxadiazole or substituted 3-aryl-5-aryl-[1,2,4]-oxadiazole can be measured. Using this assay, test compounds that bind to TIPRAIP receptor as strongly or more strongly than 3-(4-azidophenyl)-5-(3-chloro-thiophen-2-yl)-[1,2,4]-oxadiazole or substituted 3-aryl-5-aryl-[1,2,4]-oxadiazole can easily be discovered.

An alternative heterogeneous assay for use in the present invention is a biotin/avidin based assay. For examples of the various ways in which this assay can be performed in the present invention, see, e.g., Blake, R. C., et al. Anal. Biochem. 272:123-134 (1999); Cho, H. C., et al. Anal. Sciences 15:343-347 (1999); Choi, M. H., et al. Bull. Korean Chem. Soc. 22:417-420 (2001); U.S. Pat. No. 6,096,508; U.S. Pat. No. 4,863,876; and U.S. Pat. No. 4,228,237. In the present invention, avidin may be labeled with any label, preferably, avidin is fluorescently labeled or conjugated to an enzyme. Any detectably labeled enzyme can be used in the present invention specific examples include, but are not limited to, horseradish peroxidase, alkaline phophatase, β-galactosidase and glucose oxidase.

One particular embodiment of the invention employs a competitive heterogeneous biotin-avidin assay. In this assay, the test compound competes with the 3-(4-azidophenyl)-5-(3-chloro-thiophen-2-yl)-[1,2,4]-oxadiazole or the substituted 3-aryl-5-aryl-[1,2,4]-oxadiazole for the TIPRAIP binding sites. Here, biotinylated 3-(4-azidophenyl)-5-(3-chloro-thiophen-2-yl)-[1,2,4]-oxadiazole or substituted 3-aryl-5-aryl-[1,2,4]-oxadiazole is prepared. TIPRAIP bound to solid support is admixed with the biotinylated 3-(4-azidophenyl)-5-(3-chloro-thiophen-2-yl)-[1,2,4]-oxadiazole or substituted 3-aryl-5-aryl-[1,2,4]-oxadiazole and incubated for some defined period of time. 3-(4-azidophenyl)-5-(3-chloro-thiophen-2-yl)-[1,2,4]-oxadiazole or substituted 3-aryl-5-aryl-[1,2,4]-oxadiazole binds to TIPRAIP and forms a complex on the solid support. The solid support comprising biotinylated 3-(4-azidophenyl)-5-(3-chloro-thiophen-2-yl)-[1,2,4]-oxadiazole/TIPRAIP complexes or substituted 3-aryl-5-aryl-[1,2,4]-oxadiazole/TIPRAIP complexes is then admixed with a solution comprising the test compound. The mixture is allowed to incubate for some defined period of time. The test compound competes for TIPRAIP binding sites. The solid phase is then separated from the any solutions containing unbound biotinylated 3-(4-azidophenyl)-5-(3-chloro-thiophen-2-yl)-[1,2,4]-oxadiazole (or substituted 3-aryl-5-aryl-[1,2,4]-oxadiazole) or unbound test compound, and washed. The solid phase is then admixed with a composition comprising labeled avidin. The avidin binds only to the biotinylated 3-(4-azidophenyl)-5-(3-chloro-thiophen-2-yl)-[1,2,4]-oxadiazole or substituted 3-aryl-5-aryl-[1,2,4]-oxadiazole. The mixture is allowed to incubate for some defined period of time, and the amount of biotin-avidin complex is measured. The decrease in amount of biotin-avidin complex is directly related to the increase in test compound binding. Test compounds that bind TIPRAIP are candidates as apoptosis inducers.

The skilled artisan recognizes that in all of the heterogenous competitive assays described above, the ability of a test compound to effectively compete with 3-(4-Azidophenyl)-5-(3-chloro-thiophen-2-yl)-[1,2,4]-oxadiazole (or a substituted 3-aryl-5-aryl-[1,2,4]-oxadiazole) for the TIPRAIP can be ascertained by using base line values. For example, a given assay may be done with labeled 3-(4-Azidophenyl)-5-(3-chloro-thiophen-2-yl)-[1,2,4]-oxadiazole (or substituted 3-aryl-5-aryl-[1,2,4]-oxadiazole). The amount of signal associated with that label found in the separated substrate bound TIPRAIP component can be determined to give a base line value.

Then, the test compound may be introduced and a second measurement of the signal attributable to the detectable label is taken which can be compared to the base line value. The extent to which the test compound decreases the base line value is a function of the ability of the test compound to bind TIPRAIP.

C. Assays Using 3-(4-Azidophenyl)-5-(3-chloro-thiophen-2-yl)-[1,2,4]-oxadiazole or Substituted 3-aryl-5-aryl-[1,2,4]-oxadiazole Specific Antibodies

In another aspect of the invention, new candidate drugs that induce apoptosis may be identified by assaying for binding between test compounds of interest and antibodies raised against 3-(4-azidophenyl)-5-(3-chloro-thiophen-2-yl)-[1,2,4]-oxadiazole or a substituted 3-aryl-5-aryl-[1,2,4]-oxadiazole.

Antibodies to 3-(4-azidophenyl)-5-(3-chloro-thiophen-2-yl)-[1,2,4]-oxadiazole or substituted 3-aryl-5-aryl-[1,2,4]-oxadiazoles may be generated and purified using conventional, well-known methods. Such methods are described for example, in Cohler & Milstein, Nature, 256, pp. 495-497 (1975); “Antibodies-A Laboratory Manual”, E. Harlow & D. Lane, Coldspring Harbor Laboratory, pp. 55-144 (1988); C. Williams & M. Chase, in “Methods in Immunology & Immunochemistry,” Academic Press, New York, Vol. 1, Chap. 3, (1967); and S. Burchiel, in “Methods in Enzymology,” Vol. 121, Chap. 57, pp. 596-615, Academic Press, New York (1986). In general, an immunogen comprising 3-(4-azidophenyl)-5-(3-chloro-thiophen-2-yl)-[1,2,4]-oxadiazole or a substituted 3-aryl-5-aryl-[1,2,4]-oxadiazole is administered to an animal in order to elicit an immune response against the immunogen. Polyclonal antibodies generated against the immunogen are obtained from the animal antisera and are then purified using well-known methods. Monoclonal antibodies against the immunogen can be obtained from hybridoma cells using well-known methods.

Suitable immunogens for raising polyclonal antibodies include, but are not limited to, bioconjugates of 3-(4-azidophenyl)-5-(3-chloro-thiophen-2-yl)-[1,2,4]-oxadiazole or substituted 3-aryl-5-aryl-[1,2,4]-oxadiazoles. Examples of bioconjugates include, but are not limited to, conjugates between 3-(4-azidophenyl)-5-(3-chloro-thiophen-2-yl)-[1,2,4]-oxadiazole or a substituted 3-aryl-5-aryl-[1,2,4]-oxadiazole and any biological molecule, such as proteins, growth factors and cytokines. Examples include, but are not limited to proteins such as bovine hemoglobin; bovine serum albumin; growth factors such as DGF and NGF; and cytokines such as IL-2 and IL-4.

Bioconjugates are prepared by any method known to one of ordinary skill in the art. See for example, F. J. Burrows and P. E. Thorpe, “Eradication of large solid tumors in mice with an immunotoxin directed against tumor vasculature,” Proc. Natl. Acad. Sci. USA 90:8996-9000 (1993); M. Adamczyk, et al., “Characterization of Protein-Hapten Conjugates. 2. Electrospray Mass Spectrometry of Bovine Serum Albumin-Hapten Conjugates,” Bioconjugate Chem. 7:475-481 (1996); R. B. Greenwald, et al., “PEG Thiazolidiine-2-thione, a Novel Reagent for Facile Protein Modification: Conjugation of Bovine Hemoglobin,” Bioconjugate Chem. 7:638-641 (1996); U.S. Pat. Nos. 6,482,601 and 6,462,041; Maragos, C. M., Bennett, G. A., Richard, J. L., Food & Agricultural Immunology 9:3-12 (1997) and Azcona-Olivera, J. I., Abouzied, M. M., Plattner, R. D., Norred, W. P., Pestka, J. J., Appl. & Environ. Microbiol. 58:169-173 (1992). The above immunogens or bioconjugates are illustrative examples only, and any protein or polyamino acid may also be used as the carrier in a manner apparent to a person skilled in the art.

Sheep, goats and mice can be immunized with the above bioconjugates and antisera can be obtained by methods well known in the art. The antibodies may then be detectably labeled, e.g. with a radiolabel, fluorescence label, enzyme label, biotin, avidin or other label, as described above or according to methods well known in the art. Detection of binding between the test compounds of interest and the antibodies can be done by the homogenous or heterogenous methods as described above, or by any method known in the art.

VI. Cell-Based Assays

Another aspect of the present invention relates to a method of identifying TIPRAIP binding compounds using cells. Cells with altered (i.e., elevated or reduced) levels of TIPRAIP are useful for screening libraries of chemicals and compositions for TIPRAIP binding compounds that are apoptotic activating compounds which are potentially useful therapeutically as antineoplastic drugs. Such alteration can be afforded by a variety of techniques known in the art. Such techniques include antisense and RNAi methods, transfection of cells and alteration of the cellular genome.

Down regulated or reduced expression of TIPRAIP can lead to cellular resistance of apoptosis. Such resistance is manifested, for example, in a cellular culture which is non-responsive to an apoptosis activating composition. Whereas an apoptosis activating composition normally activates the caspase cascade resulting in cell death, non-responsive cells continue to thrive in the presence of such compositions. In contrast, up regulated or elevated levels of TIPRAIP may lead to cells which are more susceptible to apoptosis mediated by TIPRAIP binding compounds.

As described in greater detail below, cellular apoptosis can be monitored by following the growth rate of a cellular culture, microscopically examining cellular structure, or spectroscopically using reporter compounds. Cells with aberrant expression of TIPRAIP can be mixed with test compounds. The affect of these test compounds is compared amongst cells with elevated, reduced or normal TIPRAIP levels to determine those compounds which bind TIPRAIP and activate apoptosis.

Another aspect of the invention relates to a complex, comprising: i) a TIPRAIP; and ii) a TIPRAIP binding compound; with the proviso that the TIPRAIP binding compound is not 3-(4-azidophenyl)-5-(3-chloro-thiophen-2-yl)-[1,2,4]-oxadiazole (or substituted 3-aryl-5-aryl-[1,2,4]-oxadiazole). In addition to the above described methods, the ability of a compound to bind TIPRAIP may be determined by creating an FITC-tagged compound according to the examples described below. The TIPRAIP and bound FITC-tagged compound are isolated according to the examples described below.

A. Antisense Mediated Down Regulation of TIPRAIP

The level of TIPRAIP expression can be down regulated through the use of antisense nucleotides. An antisense nucleotide is a nucleic acid molecule that interferes with the function of DNA and/or RNA. This may result in suppression of expression. Antisense oligonucleotides also include any natural or modified oligonucleotide or chemical entity that binds specifically to a pre-mRNA or mature mRNA which results in interference or inhibition with translation of the mature mRNA or prevents the synthesis of the polypeptide encoded by the mature mRNA.

Antisense RNA sequences have been described as naturally occurring biological inhibitors of gene expression in both prokaryotes (Mizuno, T., Chou, M-Y, and Inouye, M. (1984), Proc. Natl. Acad. Sci. USA 81, (1966-1970)) and eukaryotes (Heywood, S. M. Nucleic Acids Res. , 14, 6771-6772 (1986) and these sequences presumably function by hybridizing to complementary mRNA sequences, resulting in hybridization arrest of translation (Paterson, B. M., Roberts, B. E., and Kuff, E. L., (1977) Proc. Natl. Acad. Sci. USA, 74, 4370-4374. Antisense oligodeoxynucleotides are short synthetic nucleotide sequences formulated to be complementary to a specific gene or RNA message. Through the binding of these oligomers to a target DNA or mRNA sequence, transcription or translation of the gene can be selectively blocked and the disease process generated by that gene can be halted. The cytoplasmic location of mRNA provides a target considered to be readily accessible to antisense oligodeoxynucleotides entering the cell; hence much of the work in the field has focused on RNA as a target. Currently, the use of antisense oligodeoxynucleotides provides a useful tool for exploring regulation of gene expression in vitro and in tissue culture (Rothenberg, M., Johnson, G., Laughlin. C., Green. I., Craddock, J., Sarver, N., and Cohen, J. S.(1989) J. Natl. Cancer Inst., 81:1539-1544.

The concept behind antisense therapy relies on the ability of antisense oligonucleotides to be taken up by cells and form a stable heteroduplex with the target DNA or mRNA. The end result of antisense oligonucleotide hybridization is the down regulation of the targeted protein's synthesis. Down regulation of protein synthesis by antisense oligonucleotides has been postulated to result from two possible mechanisms: 1) “hybrid arrest,” where direct blocking in pre-mRNA and/or mRNA of sequences important for processing or translation prevents full-length proteins from being synthesized; and 2) an RNase H mediated cleavage and subsequent degradation of the RNA portion of the RNA:DNA heteroduplex (Haeuptle, M. et al. (1986) Nuc. Acids Res. 14: 1427-1448; Minshull, J. and J. Hunt (1986) Nuc. Acids Res. 14: 6433-6451). Down regulation of a protein is functionally equivalent to a decrease in its activity. U.S. Pat. Nos. 5, 580,969; 5,585,479; and 5,596,090 describe antisense techniques which can be used in the down regulation of TIPRAIP.

Antisense oligonucleotides include S-oligos (nucleoside phosphorothioates) which are isoelectronic analogs of an oligonucleotide (O-oligo) in which a nonbridging oxygen atom of the phosphate group is replaced by a sulfur atom. S-oligos may be prepared by treatment of the corresponding O-oligos with 3H-1,2-benzodithiol-3-one-1,1-dioxide which is a sulfur transfer reagent. See Iyer, R. P. et al., J. Org. Chem. 55:4693-4698 (1990); and Iyer, R. P. et al., J. Am. Chem. Soc. 112:1253-1254 (1990). Antisense oligonucleotides also include such derivatives as described in U.S. Pat. Nos. 6,031,086, 5,929,226, 5,886,165, 5,693,773, 6,054,439, 5,919,772, 5,985,558, 5,595,096, 5,916,807, 5,885,970, 5,877,309, 5,681,944, 5,602,240, 5,596,091, 5,506,212, 5,521,302, 5,541,307, 5,510,476, 5,514,787, 5,543,507, 5,512,438, 5,510,239, 5,514,577, 5,519,134, 5,554,746, 5,276,019, 5,286,717, 5,264,423, as well as WO96/35706, WO96/32474, WO96/29337 (thiono triester modified antisense oligodeoxynucleotide phosphorothioates), WO94/17093 (oligonucleotide alkylphosphonates and alkylphosphothioates), W094/08004 (oligonucleotide phosphothioates, methyl phosphates, phosphoramidates, dithioates. bridged phosphorothioates, bridge phosphoramidates, sulfones, sulfates, ketos, phosphate esters and phosphorobutylamines (van der Krol et al., Biotech. 6:958-976 (1988); Uhlmann et al., Chem. Rev. 90:542-585 (1990)), W094/02499 (oligonucleotide alkylphosphonothioates and arylphosphonothioates), and WO92/20697 (3′-end capped oligonucleotides). Further, useful antisense oligonucleotides include derivatives such as S-oligonucleotides (phosphorothioate derivatives or S-oligos, see, Jack Cohen, Oligodeoxynucleotides, Antisense Inhibitors of Gene Expression, CRC Press (1989) which can be prepared, e.g., as described by Iyer et al. (J. Org. Chem. 55:4693-4698 (1990) and J. Am. Chem. Soc. 112:1253-1254 (1990)).

Antisense oligonucleotides may be coadministered with an agent which enhances the uptake of the antisense molecule by the cells. For example, the antisense oligonucleotide may be combined with a lipophilic cationic compound which may be in the form of liposomes. Methods of formulating antisense nucleotides with compositions to facilitate introduction of the antisense nucleotides into cells is disclosed, for example, in U.S. Pat. Nos. 4,897,355, 4,394,448, 4,235,871, 4,231,877, 4,224,179, 4,753,788, 4,673,567, 4,247,411, 4,814,270, 5,279,833, and 5,753,613; Published International Application Document WO 00/27795; and in published U.S. patent application No. 2002/0086849. Alternatively, the antisense oligonucleotide may be combined with a lipophilic carrier such as any one of a number of sterols including cholesterol, cholate and deoxycholic acid.

The antisense oligonucleotide may be conjugated to a peptide that is ingested by cells. Examples of useful peptides include peptide hormones, cell surface receptor ligands, antigens or antibodies, and peptide toxins. By choosing a peptide that is selectively taken up by the cells, specific delivery of the antisense agent may be effected. The antisense oligonucleotide may be covalently bound via the 5′H group by formation of an activated aminoalkyl derivative. The peptide of choice may then be covalently attached to the activated antisense oligonucleotide via an amino and sulfhydryl reactive hetero bifunctional reagent. The latter is bound to a cysteine residue present in the peptide. Upon exposure of cells to the antisense oligonucleotide bound to the peptide, the peptidyl antisense agent is endocytosed and the antisense oligonucleotide binds to the target TIPRAIP mRNA to inhibit translation. See PCT Application Publication No. PCT/US89/02363.

The antisense oligonucleotide may be at least a 15-mer that is complementary to a nucleotide molecule coding for an TIPRAIP as described herein. The antisense oligonucleotides of the present invention may be prepared according to any of the methods that are well known to those of ordinary skill in the art. The antisense oligonucleotides may be prepared by solid phase synthesis. See, Goodchild, J., Bioconjugate Chemistry, 1:165-167 (1990), for a review of the chemical synthesis of oligonucleotides. Alternatively, the antisense oligonucleotides can be obtained from a number of companies which specialize in the custom synthesis of oligonucleotides.

Methods within the scope of this invention include those wherein the antisense oligonucleotide is used in an amount which is effective to achieve inhibition of TIPRAIP expression in cells. Determination of effective amounts of each component is within the skill of the art.

B. RNA Interference (RNAi) Mediated Down Regulation of TIPRAIP

Methods employing interfering RNA (“RNAi”) use double stranded RNA that results in catalytic degradation of specific mRNAs, and can also be used to lower gene expression. See U.S. Pat. Nos. 6,458,382, 6,506,559 and 6,511,824. In this method, complementary sense and antisense RNAs derived from a portion of a gene of interest are synthesized in vitro using techniques well known in the art. The resulting sense and antisense RNAs are annealed in a buffer, and the double stranded RNA is introduced into the cell.

As described in U.S. Pat. No. 6,515,109, RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and mammals are known in the art (Fire A, et al., Nature 391:806-811 (1998); Fire, A., Trends Genet. 15:358-363 (1999); Sharp, P. A. RNA interference 2001 Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498). U.S. Pat. No. 6,511,824, also describes RNAi mediated loss-of-function phenotypes.

RNAi-mediated inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNAi-mediated inhibition in a cell line, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

RNAi mediated down regulation is affected by double stranded RNA sequences identical to a portion of the target. Accordingly, double strand RNA sequences comprise a first strand that encodes an TIPRAIP as described herein and a second strand complementary to the first strand. Alternatively, the double strand RNA comprises a first strand identical to the nucleotides described herein and a second strand complementary to the first strand. The skilled artisan recognizes that an RNA sequence is identical to a DNA sequence even though i) the ribose portion is not deoxyribose as in DNA, and ii) the nucleotide pyrimidine base thymine (usually found in DNA) is replaced by uracil. The double-stranded structure may also be formed by a single self-complementary RNA strand.

The double stranded RNA can have insertions, deletions, and single point mutations relative to the target sequence. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). In one embodiment there is more than 90% sequence identity, or even 100% sequence identity, between the inhibitory RNA and the portion of the target gene. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 25, 30, 35, 40, 45, 50, 75, 100, 125, 150, 175, 200, 225, 250, 275, 300, 400, 500, 600, 700, 800, 900, 1000 or more bases. 100% sequence identity between the RNA and the target gene is not required. Thus the invention has the advantage of being able to tolerate sequence variations that might be expected due to genetic mutation, strain polymorphism, or evolutionary divergence.

The RNA may include modifications which are well known in the art to either the phosphate-sugar backbone or the nucleosides. For example, the phosphodiester linkages of natural RNA may be modified to include at least one of a nitrogen or sulfur heteroatom. Modifications in RNA structure may be tailored to allow specific genetic inhibition. Likewise, bases may be modified to block the activity of adenosine deaminase. RNA may be produced enzymatically or by partial/total organic synthesis, any modified ribonucleotide can be introduced by in vitro enzymatic or organic synthesis.

C. Altering TIPRAIP Expression via Transfection

The skilled artisan will readily recognize that the expression level of TIPRAIP can be increased using any of the techniques described above in section IV. Expression Vectors and Transfected Cells. Altering TIPRAIP expression via transfection can also be done according to the methods of U.S. Pat. Nos. 4,980,281; 5,266,464; 5,688,655 and 5,877,007.

Such methods involve the insertion of a polynucleotide sequence encoding the TIPRAIP into an appropriate vector and the generation of cell lines which contain either (1) the expression vector alone (“control” cell lines) or (2) the expression vector containing the inserted polynucleotide (e.g., cDNA) sequence encoding the TIPRAIP. Using the appropriate vector system, recipient cell lines, and growth conditions, test cell lines can thus be generated which stably overproduce the corresponding TIPRAIP. Under the appropriate growth conditions, these cell lines will exhibit a “graded cellular response” to activators of the TIPRAIP. A graded cellular response is an increase in the phenotypic change exhibited by the cell which becomes greater with increasing expression of the TIPRAIP. It is by this specialized response that activators of apoptosis via TIPRAIP binding can be distinguished from agents that act upon other cell metabolites to effect a phenotypic change. A screening system can thus be set up whereby the control and test cell lines are propagated in defined growth conditions in tissue culture dishes (or even in experimental animals) and large numbers of compounds (or crude substances which may contain active compounds) can be screened for their ability to bind TIPRAIP and activate apoptosis.

Substances which bind to TIPRAIP and activate apoptosis may affect characteristics such as growth rate, tumorigenic potential, anti-tumorigenic potential, anti-metastatic potential, cell morphology, antigen expression, and/or anchorage-independent growth capability. Substances which specifically bind TIPRAIP and activate apoptosis may be distinguished from substances which affect cell morphology or growth by other mechanisms in that they will have a greater effect on the test lines than on the control lines.

D. Altering TIPRAIP Expression at the Genomic Level

Another aspect of the present invention involves altering the level of TIPRAIP expression at the genomic level. The gene encoding TIPRAIP is one that can be mutated to have aberrant expression, altered expression, modified expression, or mis-expression due to gene mutations, or mutations upstream or downstrean of the gene. Thus, a misexpressed protein may be one having an amino acid sequence that differs from wild-type (e.g. by amino acid substitution or deletion). These terms also include ectopic expression (e.g. by altering the normal spatial or temporal expression), over-expression (e.g. by multiple gene copies), under expression, and non-expression (e.g. by gene knockout or blocking expression that would otherwise normally occur, for example, by using antisense or RNA interference).

Such methods may involve operably associating the endogenous TIPRAIP encoded nucleotide sequence with a promoter via homologous recombination as described, for example, in U.S. Pat. No. 5,641,670, issued Jun. 24, 1997; International Publication Number WO 96/29411, published Sep. 26, 1996; International Publication Number WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired. Polynucleotide constructs are made which contain a promoter and targeting sequences, which are homologous to the 5′ non-coding sequence of endogenous TIPRAIP encoding nucleotide, flanking the promoter. The targeting sequence will be sufficiently near the 5′ end of TIPRAIP encoding nucleotide so the promoter will be operably linked to the endogenous sequence upon homologous recombination. The promoter and the targeting sequences can be amplified using PCR. The amplified promoter may contain distinct restriction enzyme sites on the 5′ and 3′ ends. The 3′ end of the first targeting sequence may contain the same restriction enzyme site as the 5′ end of the amplified promoter and the 5′ end of the second targeting sequence may contain the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

As in the methods involving transfected cells with TIPRAIP expression vectors, a graded cellular response is used to detect TIPRAIP binding agents which activate apoptosis. Specifically, the affect of a test compound on a test cell with a elevated or normal level of TIPRAIP expression is determined by comparison to the affect of a test compound on a control cell having respectively a normal or reduced level of TIPRAIP expression. As described above, test compounds which bind to TIPRAIP and activate apoptosis may affect characteristics such as growth rate, tumorigenic potential, anti-tumorigenic potential, anti-metastatic potential, cell morphology, antigen expression, cell cycle and/or anchorage-independent growth capability. Substances which specifically bind TIPRAIP and activate apoptosis may be distinguished from substances which affect cell morphology, cell cycle or growth by other mechanisms in that they will have a greater effect on the test lines than on the control lines.

E. Identifying Compounds That Activate the Caspase Cascade

The invention relates to a method for identifying potentially therapeutically effective antineoplastic compounds wherein a test compound is determined to have potential therapeutic efficacy if said caspase cascade activity is enhanced in response to the presence of said test compound, the method comprising (a) obtaining viable cultured eukaryotic cells expressing TIPRAIP (and optionally expresses a cancer phenotype) by culturing those cells in a cell growth medium under conditions which result in growth; (b) exposing the viable cultured cells to a test compound for a predetermined period of time at a predetermined temperature; (c) adding a reporter compound having at least one measurable property which is responsive to the caspase cascade; (d) measuring the caspase cascade activity of said exposed viable cultured cells by measuring said at least one measurable property of said reporter compound; and (e) wherein an increase in the measured caspase cascade activity in the presence of the test compound is an indication that the test compound is a potentially therapeutically effective antineoplastic compound.

In one embodiment, two populations of cells are screened in parallel. A first population expresses an elevated level of TIPRAIP relative to a second population. Where the first population of cells are cells that up regulate TIPRAIP, the second population of cells can be normal cells or cells which down regulate TIPRAIP (mediated, for example, by antisense nucleotides, RNAi, or altered genes). Where the first population of cells are normal cells, the second population of cells can be cells which down regulate TIPRAIP. The first and second population are separately exposed to the test compound and the reporter molecule which gives rise to a measurable property upon activation of the caspase cascade. Any increase in the reporter compound's measurable property in the first population relative to the second population is an indication that the test compound binds TIPRAIP, activates the caspase cascade, and is a potentially therapeutic antineoplastic compound.

The skilled artisan will recognize that cells with up regulated levels of TIPRAIP are expected to be more susceptible to apoptosis activated by a composition which binds to these polypeptides than are normal cells or cells which down regulate TIPRAIP. Likewise, the skilled artisan will recognize that normal cells are expected to be more susceptible to apoptosis activated by a composition which binds to these polypeptides than are cells with down regulated TIPRAIP. Hence, the first population of cells can be normal cells which neither up regulate or down regulate TIPRAIP and the second population of cells can be those which down regulate TIPRAIP.

In contrast to screening methodology using reporter compounds, the ability of a test compound to activate apoptosis can be monitored by microscopically observing changes in cellular morphology. As described in U.S. Pat. No. 6,274,309, cells can, in conjunction with the screening techniques described above, be assayed for apoptotic morphology using standard techniques well known to those of skill in the art. Among the characteristics of apoptotic morphology are cellular condensation, nuclear condensation, including chromatin condensation, and the apoptotic characteristic plasma membrane ruffling and blebbing referred to as “zeiosis” See Sanderson, C. J., 1982, in Mechanisms of Cell-Mediated Cytotoxicity, Clark, W. R. & Golstein, R., eds., Plenum Press, pp. 3-21; Godman, G. C. et al., 1075, J. Cell Biol. 64:644-667. For example, morphologic changes characteristic of nuclear apoptosis can be assayed and quantified by staining using a DNA-specific fluorochrome such as bis-benzimide (Hoechst-33258; Sigma according to standard methods. See Bose, et al., 1995, Cell 82:405-414.

As described by U.S. Pat. No. 5,932,418, DNA fragmentation is another morphological change indicative of apoptosis. DNA fragmentation may be detected with the terminal transferase assay (TUNEL; Thiry M., 1992, Highly sensitive immunodetection of DNA on sections with exogenous terminal deoxynucleotidyl transferase and non-isotopic nucleotide analogues; J. Histochem. Cytochem. 40: 419-441; Gavrieli Y, Sherman Y and Ben-Sasson SA; 1992, Identification of programmed cell death in situ-via specific labeling of nuclear DNA fragmentation; J. Cell Biol. 119:493-501). The TUNEL assay is used to detect 3′OH termini of nicked or broken DNA strands. These nicks or breaks may be generated directly by activating apoptosis. In vivo, apoptosis can be assayed via, for example, DNA terminal transferase nick-end translation, or TUNEL assay, according to standard techniques. See Fuks, Z. et al., 1995, Cancer J. 1:62-72.

Accordingly, the present invention relates to a screening method for identifying potentially therapeutically effective antineoplastic compounds by determining the ability of test compounds to alter cellular morphology in cultured eukaryotic cells expressing TIPRAIP wherein a test compound is determined to have potential therapeutic efficacy if the cellular morphology is altered in response to the presence of said test compound, the method comprising (a) obtaining cultured eukaryotic cells expressing TIPRAIP (and optionally expresses a cancer phenotype) by culturing those cells in a cell growth medium under conditions which result in growth; (b) exposing the viable cultured cells to a test compound for a predetermined period of time at a predetermined temperature; (c) microscopically examining the cellular morphology; and (d) wherein morphological changes indicative of apoptosis in the presence of the test compound is an indication that the test compound is a potentially therapeutically effective antineoplastic compound.

In another embodiment, two populations of cells are screened in parallel. A first population expresses an elevated level of TIPRAIP relative to a second population. Where the first population of cells are cells that up regulate TIPRAIP, the second population of cells can be normal cells or cells which down regulate TIPRAIP (mediated, for example, by antisense nucleotides, RNAi, or altered genes). Where the first population of cells are normal cells, the second population of cells can be cells which down regulate TIPRAIP. The first and second population are separately exposed to the test compound and the reporter molecule which gives rise to a measurable property upon activation of the caspase cascade. Any increase in the reporter compound's measurable property in the first population relative to the second population is an indication that the test compound binds TIPRAIP, activates the caspase cascade, and is a potentially therapeutic antineoplastic compound.

In contrast to screening methodology by microscopically observing changes in cellular morphology, the ability of a test compound to activate apoptosis can be monitored by following cellular culture growth. Such a screening method relates to a method of identifying potentially therapeutically effective antineoplastic compounds by determining the ability of test compounds to inhibit cellular culture growth in eukaryotic cells expressing TIPRAIP wherein a test compound is determined to have potential therapeutic efficacy if the cellular culture growth is inhibited in response to the presence of said test compound, the method comprising (a) obtaining cultured eukaryotic cells expressing TIPRAIP (and optionally expresses a cancer phenotype) by culturing those cells in a cell growth medium under conditions which result in growth; (b) exposing the cultured cells to a test compound for a predetermined period of time at a predetermined temperature; (c) following the rate of culture growth; and (d) wherein a decrease in culture growth rate in the presence of the test compound is an indication that the test compound is a potentially therapeutically effective antineoplastic compound.

In another embodiment, two populations of cells are screened in parallel. A first population expresses an elevated level of TIPRAIP relative to a second population. Where the first population of cells are cells that up regulate TIPRAIP, the second population of cells can be normal cells or cells which down regulate TIPRAIP (mediated, for example, by antisense nucleotides, RNAi, or altered genes). Where the first population of cells are normal cells, the second population of cells can be cells which down regulate TIPRAIP. The first and second population are separately exposed to the test compound and the reporter molecule which gives rise to a measurable property upon activation of the caspase cascade. Any increase in the reporter compound's measurable property in the first population relative to the second population is an indication that the test compound binds TIPRAIP, activates the caspase cascade, and is a potentially therapeutic antineoplastic compound.

Any of the methodologies discussed in this section can be performed side-by-side with control cells. Hence, in respect to the above described method employing reporter compounds, the invention also relates to a method for assaying the potency of a potentially therapeutically effective antineoplastic compound that functions as an activator of the caspase cascade in viable cultured eukaryotic cells having an intact cell membrane and expressing TIPRAIP comprising: (a) obtaining a first and a second population of viable cultured eukaryotic cells, each of which having an intact cell membrane express TIPRAIP (and optionally expresses a cancer phenotype), by culturing said eukaryotic cells in a cell growth medium under conditions which result in growth; (b) exposing the first population to a predetermined amount of a test compound for a predetermined period of time at a predetermined temperature; (c) exposing the second population to an amount of solvent that was used to dissolve the test compound for the predetermined period of time at the predetermined temperature; (d) adding to said test compound-exposed first population and said solvent-exposed second population a reporter compound having at least one measurable property which is responsive to the caspase cascade; (e) measuring said at least one measurable property of said reporter compound in said test compound-exposed first population and thereby measuring the caspase cascade activity of the test compound-exposed first population; (f) measuring said at least one measurable property of said reporter compound in said solvent-exposed second population and thereby measuring the caspase cascade activity of the solvent-exposed second population; and (g) calculating the ratio of caspase cascade activity measured for the test compound-exposed first population of cells to the caspase cascade activity measured for the solvent-exposed second population of cells to determine the relative potency of the test compound as an activator of the caspase cascade. The skilled artisan will recognize that such side-by-side screening can be modified to accommodate the above described screening methodologies which utilize microscopic observations of changes in cellular morphology, cell cycle or observations of cellular culture growth rate. Because these modified assays do not follow caspase cascade activation, they do not require addition of a reporter compound.

The caspase cascade activity measured for test compounds by this method can also be compared to that measured for compounds which are known to affect enzymes involved in the apoptosis cascade to generate a measure of the relative effectiveness of the test substance. Compounds that can be used in comparison include known activators of enzymes involved in the apoptosis cascade. Known activators, either by direct or indirect mechanisms, of enzymes involved in the apoptosis cascade include but are not limited to vinblastine, etoposide (Yoon, H. J., et al., Biochim. Biophys. Acta. 1395:110-120 (1998)) and doxorubicin (Gamen, S., et al., FEBS Lett. 417:360-364 (1997)) which are topoisomerase II inhibitors; cisplatin (Maldonado et al., Mutat. Res. 381:67-75 (1997)); chlorambucil (Hickman, J. A., Cancer Metastasis Rev. 11: 121-139 (1992)) which is an alkylating agent; and fluorouracil, an RNA/DNA anti-metabolite (Hickman, J. A., Cancer Metastasis Rev. 11: 121-139 (1992)).

In a preferred embodiment, a plurality of viable cultured cells are exposed separately to a plurality of test compounds, e.g. in separate wells of a microtiter plate. In this embodiment, a large number of test compounds may be screened at the same time.

In another aspect, the invention relates to a method for assaying the potency of a test compound to synergise with other cancer chemotherapeutic agents as an activator of the caspase cascade, comprising (a) obtaining a first and a second population of viable cultured eukaryotic cells, having an intact cell membrane and expressing TIPRAIP (and optionally expresses a cancer phenotype), by culturing the cell populations in a cell growth medium under conditions which result in growth; (b) exposing the first population to a combination of a predetermined amount of a test compound and a subinducing amount of a known cancer chemotherapeutic agent for a first predetermined period of time at a first predetermined temperature; (c) exposing the second population to an equal amount of solvent, which was used to dissolve the test compound, and a subinducing amount of a known cancer chemotherapeutic agent for said first predetermined period of time at said first predetermined temperature; (d) adding a reporter compound to the exposed first population and to the exposed second population, the reporter compound having at least one measurable property which is responsive to the caspase cascade; (e) incubating the resulting mixture of the first population, the test compound, the known cancer chemotherapeutic agent and the reporter compound for a second predetermined time period at a second predetermined temperature; (f) incubating the resulting mixture of said second population, said solvent, said known chemotherapeutic agent, and said reporter compound for a second predetermined time period at a second predetermined temperature; (g) measuring said at least one measurable property of said reporter compound in said first resulting mixture and thereby measuring the caspase cascade activity of the first population in the first resulting mixture; (h) measuring said at least one measurable property of the reporter compound in the second resulting mixture and thereby measuring the caspase cascade activity of the second population in the second resulting mixture; and (i) calculating the ratio of the caspase cascade activity of the first resulting mixture to the caspase cascade activity of the second resulting mixture to determine whether said test compound acts synergistically with the known cancer chemotherapeutic agent. The skilled artisan will recognize that such side-by-side screening can be modified to accommodate the above described screening methodologies which utilize microscopic observations of changes in cellular morphology, cell cycle do not follow caspase cascade activation, they do not require addition of a reporter compound.

The assays described in this section can also be used to screen for compositions that are selective for cell or tissue type. Such methodologies comprise side-by-side comparisons screening the affect of a given test compound on one cell or tissue type as compared to other cell or tissue types. In such an embodiment, cultures of each of the compared cell or tissue types comprise cells having elevated levels of expression of TIPRAIP. Hence, the invention also relates to a method for assaying the cell or tissue selectivity of a potentially therapeutically effective antineoplastic compound that functions as an activator of the caspase cascade in viable cultured eukaryotic cells having an intact cell membrane and expressing elevated levels of TIPRAIP comprising: (a) obtaining a first population of viable cultured eukaryotic cells, each of which having an intact cell membrane and expressing elevated levels of TIPRAIP, by culturing said eukaryotic cells in a cell growth medium under conditions which result in growth; (b) obtaining a second population of viable cultured eukaryotic cells, each of which having an intact cell membrane and expressing elevated levels of TIPRAIP by culturing said eukaryotic cells in a cell growth medium under conditions which result in growth; (c) separately exposing the first and second populations to a predetermined amount of a test compound for a predetermined period of time at a predetermined temperature; (d) adding to said first and second populations a reporter compound having at least one measurable property which is responsive to the caspase cascade; (e) measuring said at least one measurable property of said reporter compound in said first and second populations thereby measuring the caspase cascade activity of the first population relative to the second population; (f) calculating the ratio of caspase cascade activity measured for the first population of cells to the caspase cascade activity measured for the second population of cells to determine the relative cell or tissue type selectivity of the test compound as an activator of the caspase cascade, or the relative cell or tissue type selectivity of the test compound as an TIPRAIP binder. For example, the first population of cells can express a cancer phenotype that is not expressed in the second population of cells. Accordingly, this method may be used to identify compounds that while specific for cancerous cells, do not affect non-cancerous cells. The skilled artisan will recognize that such side-by-side screening can be modified to accommodate the above described screening methodologies which utilize microscopic observations of changes in cellular morphology, cell cycle or observations of changes in cellular culture growth rate. Because these modified assays do not follow caspase cascade activation, they do not require addition of a reporter compound.

The invention further relates to a method to further determine the specificity of anticancer agents by determining the ability of the agent to arrest the cell cycle during a particular phase prior to apoptosis. In this embodiment, a time course of test compound treatment determines the phase of the cell cycle arrest that precedes apoptosis. The G2M, S/G2M and G2 phases are the major phases in the cell cycle when one cell divides to become two daughter cells. The cycle starts from a resting quiescent cell (G0 phase) which is stimulated by growth factors leading to a decision (G1 phase) to replicate its DNA. Once the decision is made, the cell starts replicating its DNA (S-phase) and then into a G2 phase before finally dividing into two daughter cells. Cells which then undergo apoptosis contain fragmented DNA in amounts that are less that in the G1 phase and hence are called sub-G1. Thus, a compound leading to a G1 or G2M or S phase arrest and no apoptosis at 24 hr treatment, and leading to apoptosis at 48 hr treatment as determined by the presence of a sub-G1 peak, indicates that the test compound arrest the cell cycle at the respective stage before inducing apoptosis. See Sherr, C. J., Cancer Res. 60:3689-3695 (2000), for a discussion of cancer cell cycles.

In another aspect, the invention relates to determining the specificity of a test compound by determining at what phase the cell cycle is arrested by the test compound prior to apoptosis. Determining the specificity of a test compound to arrest the cell cycle during a particular phase prior to apoptosis comprises (a) obtaining at least one population of viable cultured cancer cells having intact cell membranes which have an elevated level of TIPRAIP from a cell growth medium under conditions conducive to growth; (b) combining the at least one population with a predetermined amount of at least one test compound dissolved in a solvent for a predetermined period of time at a predetermined temperature thereby generating a first volume; and (c) determining at what phase the cell cycle is arrested.

In this embodiment, the cells are incubated with a range of concentrations of test compound (e.g. 0.02 μM to 5 μM) for 6 h under normal growth conditions and control cultures are treated with DMSO vehicle. The cells are then treated e.g. for 20 min with 800 nM Syto 16. Cytospin preparations are then prepared and the samples are viewed by fluorescent microscopy using a fluorescein filter set. For each concentration of test compound, the number of mitotic figures are counted and expressed as a percentage of the total number of cells. Three fields from each condition are evaluated and the mean and SEM is calculated and plotted as a function of drug concentration. Another method is to simply stain the nuclei with Propidium Iodide and analyze the DNA content using a Fluorescence Activated Cell Sorter and Cell Quest Software (Becton Dickinson).

Reporter compounds, as described above, may be used as a means for measuring caspase cascade activity in the whole-cell assays of the present invention. Typical reporter compounds include fluorogenic, chromogenic or chemiluminescent compounds applied to cells or tissues containing cells at a concentration of about 0.01 nanomolar to about 0.1 molar, or an equivalent amount of a salt or prodrug thereof. A concentration of about 10 micromolar may be used.

The test compounds may be presented to the cells or cell lines dissolved in a solvent. Examples of solvents include, DMSO, water and/or buffers. DMSO may be used in an amount below 2%. Alternatively, DMSO may be used in an amount of 1% or below. At this concentration, DMSO functions as a solubilizer for the test compounds and not as a permeabilization agent. The amount of solvent tolerated by the cells must be checked initially by measuring cell viability or caspase induction with the different amounts of solvent alone to ensure that the amount of solvent has no effect on the cellular properties being measured.

Suitable buffers include cellular growth media, for example Iscove's media (Invitrogen Corporation) with or without 10% fetal bovine serum. Other known cellular incubation buffers include phoshate, PIPES or HEPES buffers. One of ordinary skill in the art can identify other suitable buffers with no more than routine experimentation.

The cells can be derived from any organ or organ system for which it is desirable to find a potentially therapeutically effective antineoplastic compound that functions as an activator of the caspase cascade in viable cultured eukaryotic cells having an intact cell membrane. Cellular genotypes for screening of test compounds include, but are not limited to, cells that are P53 negative, Bcl-2 over expressing, Bcl-xL over expressing, ataxia telengiectasia mutated (e.g. ATCC CRL 7201), multi-drug resistance (e.g.

P-glycoprotein over expressing, ATCC CRL-1977), DNA mismatch repair deficiency (e.g., defects in hMSH2, hMSH3, hMSH6, hPMS2, or hPMS1), HL-60 cells (ATCC CCL-240), SH-SY5Y cells (ATCC CRL-2266), and Jurkat cells (ATCC TIB-152), surviving over expressing (e.g. ATCC CCL-185), bcr/abl mutated (eg ATCC CCL-243), p16 mutated, Brcal mutated (e.g. ATCC CRL-2336), or Brca2 mutated. These and other cells may be obtained from the American Type Culture Collection, Manassas, Va.

Suitable solubilizers may be used for presenting reporter compounds to cells or cell lines. Solubilizers include aqueous solutions of the test compounds in water-soluble form, for example as water-soluble salts. The test compounds may be dissolved in a buffer solution containing 20% sucrose (Sigma) 20 mM DTT (Sigma), 200 mM NaCl (Sigma), and 40 mM Na PIPES buffer pH 7.2 (Sigma).

Inasmuch as the caspase cascade takes place in the intracellular environment, measures may be undertaken to enhance transfer of the reporter compound across the cell membrane. This can be accomplished with a suitable permeabilization agent. Permeabilization agents include, but are not limited to, NP-40, n-octyl-O-D-glucopyranoside, n-octyl-O-D-thioglucopyranoside, taurocholic acid, digitonin, CHAPS, lysolecithin, dimethyldecylphosphine oxide (APO-10), dimethyldodecylphosphine oxide (APO-12), N,N-bis-(3-D-gluconamidopropyl)cholamide (Big Chap), N,N-bis-(3-D-gluconamidopropyl)deoxycholamide (Big Chap, deoxy), BRIG-35, hexaethyleneglycol (C10E6), C10E8, C12E6, C12E8, C12E9, cyclohexyl-n-ethyl-O-D-maltoside, cyclohexyl-n-hexyl-O-D-maltoside, cyclohexyl-n-methyl-O-D-maltoside, polyethylene glycol lauryl ether (Genapol C-100), polyethylene glycol dodecyl ether (Genapol X-80), polyoxyethylene isotridecyl ether (Genapol X-100), n-decanoylsucrose, n-decyl-O-D-glucopyranoside, n-decyl-O-D-maltopyranoside, n-decyl-O-D-thiomaltoside, n-dodecanoylsucrose, n-dodecyl-O-D-glucopyranoside, n-dodecyl-O-D-maltoside, n-heptyl-O-D-glucopyranoside, n-heptyl-O-D-thioglucopyranoside, n-hexyl-O-D-glucopyranoside, n-nonyl-O-D-glucopyranoside, n-octanoylsucrose, n-octyl-O-D-maltopyranoside, n-undecyl-O-D-maltoside, n-octanoyl-O-D-glucosylamine (NOGA), PLURONIC⁷ F-127, and PLURONIC⁷ F-68.

The cell lines are exposed to a predetermined amount of test compounds at concentrations in the range from about 1 picomolar to about 1 millimolar, or about 1-10 micromolar. The predetermined period of time may be about 1 hour to less than about 48 hours, or 3-48 hours, or 3, 5, 24, or 48 hours. The predetermined temperature may be about 4° C. to about 50° C., or about 37° C.

F. Measuring the Potency of Caspase Cascade Activation

Using a fluorescent plate reader, an initial reading (T=0) is made immediately after addition of the reporter reagent solution, employing excitation and emission at an appropriate wavelength (preferably excitation at 485 nm and emission at 530 nm) to determine the background absorption and/or fluorescence of the control sample. After the incubation, the absorption and/or fluorescence of the sample is measured as above (e.g., at T=3 hr).

Sample Calculation:

The Relative Fluorescence Unit values (RFU) are used to calculate the potency of the test compounds as follows:
RFU_{(T=}3 hr)−RFU_{(T=}0)=Net RFU
The potency of caspase cascade activation is determined by the ratio of the Net RFU value for a test compound to that of control samples as follows:

$$\frac{Net RFU of test compound}{Net RFU of control sample} = Ratio$$

Preferred test compounds are those indicating a ratio of 2 or greater and most preferably with a measured ratio greater than a statistically significant value calculated as (Ave Control RFU+4×SD_{Control})/(Ave Control RFU) for that run.

Examples of high throughput instrumentation which can be used according to the present invention are well known in the art. Non-limiting examples of such instruments include ImageTrak® (Packard BioScience), the FLIPR® system, Spectramax Gemini or FMax (Molecular Devices Corporation, Sunnyvale, Calif.), VIPR™ II Reader (Aurora Biosciences Corporation, San Diego, Calif.), Fluoroskan II (GMI, Inc., Albertville, Minn.), Fluoroskan Ascent (Labsystems, Franklin, Mass.), Cytofluor or Cytofluor 4000 (Perkin Elmer Instruments), Cytofluor 2300 (Millipore, FLx800TBID, FLx800TBIDE, ELx808, ELx800, FL600 (Bio-Tek Instruments), Spectrafluora, Spectrofluora Plus, Ultra or Polarion (Tecan AG), MFX (Dynex Technologies, Chantilly, Va.), Fluoro Count (Packard Instruments Co.), NOVOstar, POLARstar Galaxy or FLUOstar Galaxy (BMG Lab Technologies GmbH), Fluorolite 1000 (Dynex Technologies), 1420 Victor 2 (EG&G Wallac, Inc., also available through PerkinElmer), and Twinkle LB 970 (Berthold Technologies GmbH & Co.). VII. Diagnosis and Prognosis

It is believed that certain tissues in mammals with certain diseases (e.g. cancer or autoimmune diseases) express significantly altered (enhanced or decreased) levels of TIPRAIP and mRNA encoding TIPRAIP when compared to tissues of a corresponding “standard” mammal, i.e., a mammal of the same species not having the disease. Further, it is believed that altered levels of TIPRAIP can be detected in certain body fluids (e.g., sera, plasma, urine, and spinal fluid) from mammals with the disease when compared to sera from mammals of the same species not having the disease. Thus, the invention provides a diagnostic method useful during diagnosis, which involves assaying the expression level of the gene encoding TIPRAIP in mammalian cells or body fluid and comparing the gene expression level with a standard TIPRAIP gene expression level, whereby an increase or decrease in the gene expression level over the standard is indicative of the disease.

Where a diagnosis has already been made according to conventional methods, the present invention is useful as a prognostic indicator, whereby patients exhibiting lowered TIPRAIP gene expression will experience a worse clinical outcome in response to administration of an TIPRAIP binding compound relative to patients expressing TIPRAIP at a normal level.

By “assaying the expression level of the gene encoding TIPRAIP” is intended qualitatively or quantitatively measuring or estimating the level of TIPRAIP or the level of the mRNA encoding TIPRAIP in a first biological sample either directly (e.g., by determining or estimating absolute protein level or mRNA level) or relatively (e.g., by comparing to the TIPRAIP level or mRNA level in a second biological sample). The TIPRAIP level or mRNA level in the first biological sample may be measured or estimated and compared to a standard TIPRAIP level or mRNA level, the standard being taken from a second biological sample obtained from an individual not having the cancer. As will be appreciated in the art, once a standard TIPRAIP level or mRNA level is known, it can be used repeatedly as a standard for comparison.

By “biological sample” is intended any biological sample obtained from an individual, cell line, tissue culture, or other source which contains TIPRAIP or mRNA. Biological samples include mammalian body fluids (such as sera, plasma, urine, synovial fluid and spinal fluid) which contain secreted TIPRAIP, and ovarian, prostate, heart, placenta, pancreas liver, spleen, lung, breast and umbilical tissue.

Total cellular RNA can be isolated from a biological sample using the single-step guanidinium-thiocyanate-phenol-chloroform method described in Chomczynski and Sacchi, Anal. Biochem. 162:156-159 (1987). Levels of mRNA encoding TIPRAIP are then assayed using any appropriate method. These include Northern blot analysis, (Harada et al., Cell 63:303-312 (1990) S1 nuclease mapping, (Fijita et al., Cell 49:357-367(1987)) the polymerase chain reaction (PCR), reverse transcription in combination with the polymerase chain reaction (RT-PCR) (Makino et al., Technique 2:295-301 (1990), and reverse transcription in combination with the ligase chain reaction (RT-LCR).

Assaying TIPRAIP levels in a biological sample can be done using antibody-based techniques. For example, TIPRAIP expression in tissues can be studied with classical immunohistological methods. (Jalkanen, M., et al., J. Cell. Biol. 101:976-985 (1985); Jalkanen, M., et al., J. Cell. Biol. 105:3087-3096 (1987)).

Other antibody-based methods useful for detecting TIPRAIP gene expression include immunoassays, such as the enzyme linked immunosorbent assay (ELISA) and the radioimmunoassay (RIA).

Suitable labels are known in the art and include enzyme labels, such as, Glucose oxidase, and radioisotopes, such as iodine (¹²⁵I, ¹²¹I), carbon (¹⁴C), sulfur (³⁵S), tritium (³H), indium ( 112In), and technetium (⁹⁹Tc), and fluorescent labels, such as fluorescein and rhodamine, and biotin.

VIII. Rational Drug Design Using TIPRAIP Structure

As described in U.S. Pat. No. 6,150,088, a structure-based approach can be used, along with available computer-based design programs, to identify or design a drug which will fit into, line or bind a cavity or pocket of TIPRAIP.

For example, this method can be carried out by comparing the members of the chemical library with the crystal structure of a TIPRAIP using computer programs known to those of skill in the art (e.g., Dock, Kuntz, I. D. et al., Science, 257:1078-1082 (1992); Kuntz, I. D. et al., J. Mol. Biol., 161:269 (1982); Meng, E. C., et al., J. Comp. Chem., 13: 505-524 (1992) or CAVEAT). In this method, the library of molecules to be searched can be any library, such as a database (i.e., online, offline, internal, external) which comprises crystal structures, coordinates, chemical configurations or structures of molecules, compounds or drugs to be assessed or screened for their ability to bind a TIPRAIP. For example, databases for drug design, such as the Cambridge Structural Database (CSD), which includes about 100,000 molecules whose crystal structures have been determined or the Fine Chemical Director (FCD) distributed by Molecular Design Limited (San Leandro, Calif.) can be used. See Allen, F. H., et al., Acta Crystallogr. Section B, 35:2331 (1979). In addition, a library, such as a database, biased to include an increased number of members which comprise indole rings, hydrophobic moieties and/or negatively-charged molecules can be used.

A drug or molecule which binds or fits into a cavity or pocket on the surface of a TIPRAIP, can be used alone or in combination with other drugs (as part of a drug cocktail) to prevent, ameliorate or treat conditions responsive to induction of apoptosis. A drug designed or formed by a method described herein is also the subject of this invention.

IX. Screening for Apoptosis Inducing Compounds by Monitoring Gene Expression Profile

Test compounds can also be screened for their ability to induce apoptosis by monitoring mRNA gene expression level in cells, tissues, unicellular organisms or multicellular organisms. For example, after treating a cell with one or more test compounds, the expression levels of certain mRNAs can be assayed using various techniques well known to the skilled artisan, including quantitative PCR. A test compound can be identified as a potential anti-cancer agent depending on whether the expression levels (or the ratios there between) of certain mRNAs increase or decrease.

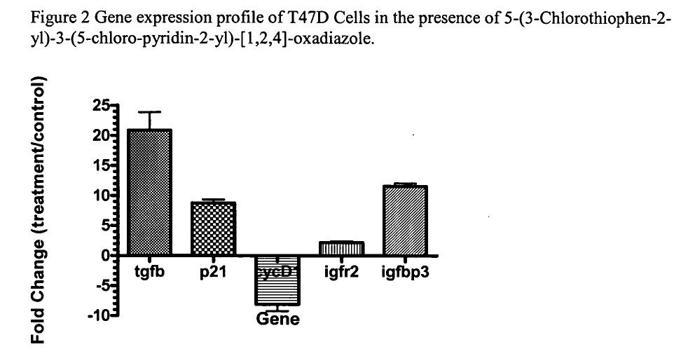

For example, an increase in mRNA encoding transforming growth factor beta (TGFβ, e.g. NCBI accession no. AB000584), cyclin-dependent kinase inhibitor 1A (p21, e.g. NCBI accession no. NM_{—}000389), insulin-like growth factor 2 receptor (IGF2R, e.g. NCBI accession no. NM_{—}000876), or insulin-like growth factor binding protein 3 (IGFBP3, e.g. NCBI accession no. NM_{—}000598) is characteristic of a test compound capable of inducing apoptosis. Such compounds induce apoptosis and are potential anti-cancer agents. A decrease in mRNA encoding cyclin D1 (CycD1, e.g. NCBI accession no. BC000076) is also characteristic of a test compound capable of compound can be screened for increasing or decreasing the expression level of one or more of the above described mRNAs. Alternatively, a test compound can be screened for altering the expression level ratio between two mRNAs. Moreover, the skilled artisan recognizes that mRNA screening is not limited to the above described mRNAs identified by the exemplary NCBI accession numbers. Rather, the skilled artisan recognizes that mutants, variations, splice variants or other modified or species-specific versions of the above described mRNAs can also be used in the screening method. A non-limiting example of such a screening method is described in Example 7, below, and in FIG. 2.

X. Screening for Apoptosis Inducing Compounds by Monitoring Interactions Between Biological Components

Test compounds can also be screened for their ability to induce apoptosis by monitoring their ability to disrupt or interfere with the ability of two or more biological components (e.g. two or more proteins) to interact with each other. For example, the ability of a test compound to disrupt or interfere with the interaction between tail interacting protein-47 (TIP47, or cargo selection protein TIP47, e.g. NCBI accession no. AAC39751) and insulin-like growth factor 2 receptor (IGF2R, e.g. NCBI accession no. NP_{—}000867) can be used as an indication as to whether the test compound induces apoptosis. The ability of these two proteins to bind each other can be assessed according to the techniques described by Krise, J. P. et al., “Quantitative Analysis of Tip47-Receptor Cytoplasmic Domain Interactions,” J. Biol. Chem. 275(33): 25188-25193 (2000); or Orsel, J. G. et al., “Recognition of the 300-kDa mannose 6-phosphate receptor cytoplasmic domain by 47-kDa tail-interacting protein,” Proc. Natl. Acad. Sci. 97(16): 9047-9051 (2000), both of which are wholly incorporated by reference herein.

Test compounds which disrupt TIP47 binding to IGF2R are capable of inducing apoptosis and are potential anti-cancer agents. The skilled artisan recognizes that TIP47 binding to IGF2R is not limited to the above described. proteins identified by the exemplary NCBI accession numbers. Rather, the skilled artisan recognizes that mutants, variations, derivatives and species-specific versions of the above described proteins can also be used in the screening method. In addition, the skilled artisan will recognize that the interaction between other proteins or biological components can also be assessed to ascertain whether a test compound is capable of inducing apoptosis.

### XI. EXAMPLES

### Example 1

### 5-(3-Chlorothiophen-2-yl)-3-(4-chloro-3-(N-methyl-N-(4-butyryl-aminoalkyl-agarose)-amino)-phenyl)-[1,2,4]-oxadiazole

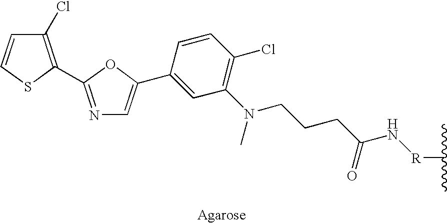

a. 4-Chloro-3-(N-methyl-N-(4-butyric acid methyl ester)-amino)-benzonitrile: A solution of 4,4-Dimethoxy-butyric acid methyl ester (5.0 g, 30.8 mmol), 1.2 M hydrochloric acid solution (12 mL), and acetone (100 mL) was stirred at room temperature for 20 minutes. The solution was concentrated by rotary evaporation and the residue was partitioned between water (50 mL) and dichloromethane (3×60 mL). The combined dichloromethane layers were dried over sodium sulfate and were concentrated by rotary evaporation. To the residue was added dichloromethane (150 mL), 3-amino-4-chloro-benzonitrile (1.19 g, 7.83 mmol), acetic acid (1.8 mL, 31 mmol), and sodium triacetoxyborohydride (6.74 g, 31.8 mmol), and the solution was stirred at room temperature for 15 hours. The solution was concentrated by rotary evaporation and was partitioned between ethyl acetate (100 mL) and water (50 mL). The ethyl acetate layer was concentrated by rotary evaporation and the residue was purified by flash column chromatography (7:2 hexanes/ethyl acetate) to yield 2.21 g of a white solid. To the white solid was added glacial acetic acid (80 mL), paraformaldehyde (2.34 g, 78.1 mmol), and sodium cyanoborohydride (1.82 g, 6.83 mmol); and the solution was stirred for 17 hours at room temperature. The solution was partitioned between ethyl acetate and saturated sodium bicarbonate solution (1200 mL), and the ethyl acetate layer was concentrated by rotary evaporation. The residue was purified by flash column chromatography (5:1 hexanes/ethyl acetate) to yield 1.82 g (87%) of a cololess oil. ¹H NMR (CDCl₃): 7.43 (d, J =8.25 Hz, 1H), 7.29 (d, J=1.64 Hz, 1H), 7.21 (dd, J_{BA}=8.24 Hz, J_{BX}=1.93, 1H), 3.68 (s, 3H), 3.08 (t, J=7.42 Hz, 2H), 2.80 (s, 3H), 2.39 (t, J=7.28 Hz, 2H), 1.93 (d, J=7.35 Hz, 2H).

b. 4-Chloro-3-(N-methyl-N-(4-butyric acid methyl ester)-amino)-benzamideoxime: A solution of 4-chloro-3-(N-methyl-N-(4-butyric acid methyl ester)-amino)-benzonitrile (1.81 g, 6.79 mmol), hydroxylamine (420 μL, 6.85 mmol), and ethanol (11.0 mL) was stirred for 1.25 hours at room temperature. Hydroxylamine (420 μL, 6.85 mmol) was added to the solution and it was stirred for 1.5 hours. Hydroxylamine (420 μL, 6.85 mmol) was added to the solution and it was stirred for 1.75 hours. The solution was partitioned between ethyl acetate (100 mL) and water (3×75 mL). The ethyl acetate layer was concentrated by rotary evaporation and was purified by flash column chromatography (2:1 hexanes/ethyl acetate) to yield 1.66 g (81%) of a colorless oil. ¹H NMR (DMSO-d₆): 7.46 (d, J=1.65 Hz, 1H), 7.38 (d, J=8.24 Hz, 1H), 7.30 (dd, J_{BA}=8.24 Hz, J_{BX}=1.93, 1H), 3.57 (s, 3H), 2.99 (t, J=7.14 Hz, 2H), 2.69 (s, 3H), 2.35 (t, J=7.28 Hz, 2H), 1.76 (d, J=7.21 Hz, 2H).

c. 5-(3-Chlorothiophen-2-yl)-3-(4-chloro-3-(N-methyl-N-(4-butyric acid methyl ester)-amino)-phenyl)-[1,2,4]-oxadiazole: A solution 4-chloro-3-(N-methyl-N-(4-butyric acid methyl ester)-amino)-benzamideoxime (1.65 g, 5.49 mmol), 3-chloro-thiophene-2-carbonyl chloride (995 mg, 5.49 mmol), and pyridine (13.0 mL) was stirred for 5 minutes under argon at room temperature. The solution was then refluxed for 1.6 hours under argon in an oil bath at 118° C. The solution was cooled to room temperature and it was partitioned between water (100 mL) and ethyl acetate (100 mL). The ethyl acetate layer was concentrated by rotary evaporation and the product was purified by flash column chromatography (6:1 hexanes/ethylacetate) to yield 2.16 g (93%) of the title compound as a colorless oil. ¹H NMR (CDCl₃): 7.84 (d, J=1.93 Hz, 1H), 7.74 (dd, J_{BA}=8.24 Hz, J_{BX}=1.92, 1H), 7.61 (d, J=5.22 Hz, 1H), 7.48 (d, J=8.24 Hz, 1H), 7.13 (d, J=5.50 Hz, 1H), 3.68 (s, 3H), 3.13 (t, J=7.28 Hz, 2H), 2.85 (s, 3H 2.42 (t, J=7.42 Hz, 2H), 1.96 (d, J=7.35 Hz, 2H).

d. 5-(3-Chlorothiophen-2-yl)-3-(4-chloro-3-(N-methyl-N-(4-butyric acid)-amino)-phenyl)-[1,2,4]-oxadiazole: A solution of lithium hydroxide (280 mg, 6.67 mmol) and water (5.0 mL) was added to a solution of 5-(3-chlorothiophen-2-yl)-3-(4-chloro-3-(N-methyl-N-(4-butyric acid methyl ester)-amino)-phenyl)-[1,2,4]-oxadiazole (2.03 g, 4.76 mmol), and tetrahydrofuran (55 mL) and the solution was stirred for 21 hours at room temperature. Ethanol (10 mL) was added and the solution was stirred for 10.5 hours. Then 3 M sodium hydroxide (1.05 mL, 3.15 mmol) and ethanol (3 mL) were added and the solution was stirred for 30 minutes. The solution was acidified to pH 3 and was extracted with ethyl acetate (100 ML). The ethyl acetate layer was concentrated by rotary evaporation and the product was purified by flash column chromatography (dichloromethane: ethyl acetate, 1:2) to yield 1.65 g (84%) of the title compound as a white solid. ¹H NMR (CDCl₃): 7.86 (d, J=1.92 Hz, 1H), 7.76 (dd, J_{BA}=8.24 Hz, J_{BX}=1.92, 1H), 7.62 (d, J=5.22 Hz, 1H), 7.49 (d, J=8.24 Hz, 1H), 7.14 (d, J=5.49 Hz, 1H), 3.16 (t, J=7.01 Hz, 2H), 2.85 (s, 3H), 2.48 (t, J=7.27 Hz, 2H), 1.94 (d, J=7.21 Hz, 2H).

e. 5-(3-Chlorothiophen-2-yl)-3-(4-chloro-3-(N-methyl-N-(4-butyric acid N-hydroxysuccinimide ester)-amino)-phenyl)-[1,2,4]-oxadiazole: A solution of 5-(3-chlorothiophen-2-yl)-3-(4-chloro-3-(N-methyl-N-(4-butyric acid)-amino)-phenyl)-[1,2,4]-oxadiazole (1.64 g, 3.97 mmol), N-hydroxysuccinimide (688 mg, 5.98 mmol), dicyclohexyl-carbodiimide (1.22 g 5.89 mmol). and dichloromethane (60 mL) was stirred for 1.5 hours at room temperature and the solution was filtered. The filtrate was concentrated to dryness by rotary evaporation. The product was purified by column chromatography (9:1 dichloromethane/ethyl acetate) to yield 1.85 g (91%) of the title compound as a white solid. ¹H NMR (CDCl₃): 7.86 (d, J=1.92 Hz, 1H), 7.76 (dd, J_{BA}=8.24 Hz, J_{BX}=1.93, 1H), 7.61 (d, J=5.22 Hz, 1H), 7.49 (d, J=8.24 Hz, 1H), 7.13 (d, J=5.22 Hz, 1H), 3.20 (t, J=7.14 Hz, 2H), 2.85 (m, 7H), 2.77 (t, J=7.41 Hz, 2H), 2.07 (d, J=7.28 Hz, 2H).

f. 5-(3-Chlorothiophen-2-yl)-3-(4-chloro-3-(N-methyl-N-(4-butyryl-aminoalkyl-agarose)-amino)-phenyl)-[1,2,4]-oxadiazole: Biorad Affi Gel 102 Gel aminoalkyl agarose (10 ml, 0.12 mmol) was placed in a solid phase reaction vessel and was rinsed with 1:1 dimethylsuloxide/water (1×20 mL) and dimethyl sulfoxide (3×30 mL). 5-(3-chlorothiophen-2-yl)-3-(4-chloro-3-(N-methyl-N-(4-butyric acid N-hydroxysuccinimide ester)-amino)-phenyl)-[1,2,4]-oxadiazole (105.9 mg, 0.208 mmol) and dimethylsulfoxide (22.0 mL) were added to the reaction vessel and the vessel was shaken mildly for 14.5 hours at room temperature. The solution flushed and the reaction vessel was rinsed with dimethylsulfoxide (3×20 mL) and 30% aqueous ethanol (5×20 mL). The agarose beads were then suspended in 30% aqueous ethanol.

### Example 2

### 5-(3-Chlorothiophen-2-yl)-3-(4-chloro-3-(N-methyl-N-(2-acetyl-aminoalkyl-agarose)-amino)-phenyl)-[1,2,4]-oxadiazole

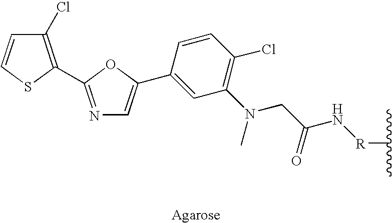

The title compound was prepared by a procedure similar to Example 1 from reaction of Biorad Affi Gel 102 Gel aminoalkyl agarose with 5-(3-chlorothiophen-2-yl)-3-(4-chloro-3-(N-methyl-N-(2-acetic acid N-hydroxysuccinimide ester)-amino)-phenyl)-[1,2,4]-oxadiazole.

### Example 3

### 3-(3,5 -Ditritium-4-azidophenyl)-5-(3-chloro-thiophen-2-yl)-[1,2,4]-oxadiazole

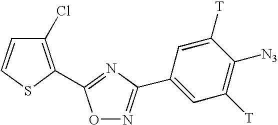

a. 3-(4-Azidophenyl)-5-(3-chloro-thiophen-2-yl)-[1,2,4]-oxadiazole: A mixture of 3-(4-aminophenyl)-5-(3-chloro-thiophen-2-yl)-[1,2,4]-oxadiazole (15.5 mg, 0.05 mmol) in acetic acid (2 mL) and conc. sulfuric acid (0.3 mL) was added sodium nitrite (3.8 mg, 0.055 mmol) in water (0.5 mL). The mixture was stirred vigorously at 0-5° C. for 20 min, then sodium azide (3.6 mg, 0.055 mmol) in water (0.5 mL) was added. It was stirred at 0-5° C. for 3 h and then poured into ice water (30 mL). The resultant mixture was extracted with ethyl acetate (3×10 mL). The organic layer was washed with water, dried over anhydrous sodium sulfate, and evaporated. The crude residue was purified by flash chromatography to yield 16 mg (100%) of the title compound. ¹H NMR (CDCl₃): 8.18 (d, J=8.7 Hz, 2H), 7.63 (d, J=5.4 Hz, 1H), 7.18 (d, J=8.7 Hz, 1H), 7.16 (d, J=5.4 Hz, 2H).

b. 3-(3,5-Ditritium-4-azidophenyl)-5-(3-chloro-thiophen-2-yl)-[1,2,4]-oxadiazole: The T-labeled azido compound was prepared by a procedure similar as the non-labeled compound by using 3-(3,5-ditritium-4-aminophenyl)-5-(3-chloro-thiophen-2-yl)-[1,2,4]-oxadiazole as the starting materials. 3-(3,5-Ditritium-4-aminophenyl)-5-(3-chloro-thiophen-2-yl)-[1,2,4]-oxadiazole was prepared by reaction of 3-(4-amino-3,5-diiodophenyl)-5-(3-chloro-thiophen-2-yl)-[1,2,4]-oxadiazole with T₂ in the presence of a metal catalyst. The T-labeled azido compound was purified by HPLC, with chemical and radiochemical purity of >98%, and specific activity of 40-50 Ci/mmol.

### Example 4

### Isolation and Identification of Tail Interacting Protein

Isolation of Tail Interacting Protein from Cell Extracts by Photo-affinity Radiolabeling: T47D breast cancer cell line was grown in RPMI 1640 medium containing 25 mM Hepes and L-glutamine (Gibco) supplemented with 10% FCS and penicillin/streptomycin. 8×10⁶ T47D cells in 25 mL medium were plated on a 100 mm dish and grown overnight in RPMI medium supplemented with 10% FCS and penicillin/streptomycin. Cells were scraped with Cell lifter (Fisher) into a conical tube and centrifuged for 5 minutes at 450×g. Cells were washed one time with 1 mL PBS (1,160×g for 3 minutes) and then resuspended in 0.25 mL Cell Lysis Buffer (CLB) (10 mM HEPES, pH 7.2, 10 mM NaCl, 1 mM KH₂PO₄, 5 mM NaHCO₃, 1 mM CaCl₂, 0.5 mM MgCl₂, 5 mM EDTA) plus 0.1% Protease Inhibitor Cocktail (Sigma). Cells were allowed to swell 5 minutes at room temperature and then homogenized using Dounce homogenizer and Type A pestle (tight) 50 times on ice. After centrifugation at 2,200×g, for 5 minutes, 4° C., the supernatant was spun at 108,000×g, for 40 minutes at 4° C. This supernatant is T47D cytosol. Protein concentration is determined by BioRad DC assay.

300 μg T47D cytosol in 100 μL CLB was added in the well of a 96-well plate. 200 nM 3-(3,5-ditritium-4-azidophenyl)-5-(3-chloro-thiophen-2-yl-[1,2,4]-oxadiazole (Example 3) (50 Ci/mmol) was added to the well and allowed to mix on a rocker at room temperature for 30 minutes. The plate was then exposed to a short wavelength UV Source (UVG-54, Ultra Violet Product.Inc) (254 nm) for 10 minutes at a distance of 3.5 cm from the plate. A duplicate sample was prepared in parallel but without radiolabeled compound and not irradiated.

For two-dimensional gel analysis, samples were concentrated in a YM-30 Microcon concentrator (Millipore) according to the manufacturer's instructions. 10 μL (˜300μg) of protein sample was added to pH 4-7/6-9 rehydration buffer (Invitrogen Corporation) with 20 mM DTT to a final volume of 155 μL. 155 μL of rehydration buffer was loaded into the sample loading well of the IPG Runner (Invitrogen Corporation) cassette. pH 3-10 non-linear Zoom strip was inserted into the sample well of the cassette. The strip was incubated at room temperature overnight. Cassette was placed in the IPG Runner and IEF (1^{st} dimension) performed at 500 V for 4 hours, with a current limit of 1 mA per strip and a power limit of 0.5 W per strip. Following IEF, strips were placed into 15 mL conical tubes with 5 mL 1×NuPAGE LDS sample buffer (Invitrogen Corporation) with Sample Reducing Agent (Invitrogen Corporation) and incubated for 15 minutes at room temperature. A second incubation was done in 5 mL 125 mM alkylating solution (116 mg iodoacetamide/5 mL 1×NuPAGE LDS sample buffer) for 15 minutes at room temperature. SDS PAGE (2_{nd} dimension) was done by cutting-off 0.7 cm at the basic end of the strips, then inserting strip into 2-D well of a 10% Tris-Glycine gel (Invitrogen Corporation) and overlaying with a 0.5% agarose solution. Strips were then run for 60 minutes at 30 mA per gel, stained with 1% Coomassie Brilliant Blue in 40% methanol, 7.5% acetic acid overnight at room temperature. Gels were destained in several changes of destainer (40% methanol, 7.5% acetic acid), incubated in Amplify (Amersham) for 30 minutes at room temperature and then dried down at 80° C. for 2 hours on a gel dryer (Savant). Dried gels were put on Hyperfilm (Amersham) and placed at −80 ° C. Film was developed 5-7 days later.

The duplicate 2-D gel of non-radiolabeled lysate, not treated with 3-(3,5-ditritium-4-azidophenyl)-5-(3-chloro-thiophen-2-yl)-[1,2,4]-oxadiazole, was left in destain solution until autoradiography film was developed. The film which showed a single radiolabeled spot (approximately 50 kDa, pI 5.3) was oriented with the duplicate non-radiolabeled lysate gel to locate the position of the protein on the non-radiolabeled gel. The protein spot was excised from the gel with a sterile Pasteur pipette and placed in a tube for tryptic digestion.

Trypsin digestion: The gel slice was further destained in 30% MeOH until the background was nearly clear. The gel slice was incubated for at least an hour in 500 μL of 100 mM ammonium bicarbonate. Then 150 μL of 100 mM ammonium bicarbonate and 10 μL of 45 mM DTT were added and incubated at 60° C. for 30 minutes. Samples were cooled to room temperature and 10 μL of 100 mM iodoacetamide was added and the sample incubated for 30 minutes in the dark at room temperature. The solution was removed and discarded and 500 μL of 50% acetonitrile and 50% 100 mM ammonium bicarbonate, pH 8.9, were added and the sample incubated with shaking for 1 hour at room temperature. The gel was removed, cut into 2-3 pieces and transferred to a 200 μL Eppendorf tube. 50 μL acetonitrile was added for 10-15 minutes and then removed. The gel slices were dried in a Savant rotatory evaporator. The gel pieces were incubated with 10 μL of 25 mM ammonium bicarbonate containing Promega modified trypsin (sequencing grade) at a concentration such that a substrate to enzyme ratio of 10:1 had been achieved (typically 0.1 μg). The protein amounts were estimated from the staining intensity of the gel. After 10-15 minutes, 10-20 μL 25 mM ammonium bicarbonate was added to cover the gel pieces and incubated overnight at 37° C. The samples were then frozen at −20° C. until analysis by peptide mass sequencing.

LC-MS/MS peptide sequencing and protein identification: This was carried out by standard procedures at mass spectrometry sequencing facility: Centre Proteomique de l'Est du Québec, Ste-Foy, Quebec, Canada or equivalent facilities. In short, the samples were run on LC-MS/MS ion trap instruments and the parent and fragments were analyzed for mass to charge ratios. From the degradation fragments, a peptide sequence was deduced which is generally within 1 amu (atomic mass unit) of the predicted mass. These sequences were then compared to peptide sequences in the gene sequence or protein sequence databases. Identity of peptide sequence with predicted tryptic fragments from gene sequences indicates the peptide as part of the gene. The size of the peptide matched and/or the number of matched peptides confirm the identity of the protein.

The following lists the experimentally deduced peptide sequences having the closest fitting calculated molecular weights. An NCBI Blast search (accessible at http://www.ncbi.nlm.nih.gov/BLAST/) using these peptides revealed that they are a part of SEQ ID NO.: 7.

| | |
|---|---|
| Amino acid Sequence | AA Positions |

| | |
|---|---|
| DTVATQLSEAVDATR | amino acids 141-155 of |
| | SEQ ID NO.: 7 |
| GLDKLEENLPILQQPTEK | amino acids 99-116 of |
| | SEQ ID NO.: 7 |
| IATSLDGFDVASVQQQR | amino acids 214-230 of |
| | SEQ ID NO.: 7 |
| LEPQIASASEYAHR | amino acids 85-98 of |
| | SEQ ID NO.: 7 |
| LGQMVLSGVDTVLGK | amino acids 181-195 of |
| | SEQ ID NO.: 7 |
| QEQSYFVR | amino acids 231-238 of |
| | SEQ ID NO.: 7 |
| QLQGPEKEPPKPEQVESR | amino acids 308-325 of |
| | SEQ ID NO.: 7 |
| SEEWADNHLPLTDAELAR | amino acids 196-213 of |
| | SEQ ID NO.: 7 |
| SVVTGGVQSVMGSR | amino acids 167-180 of |
| | SEQ ID NO.: 7 |
| TLTAAAVSGAQPILSK | amino acids 69-84 of |
| | SEQ ID NO.: 7 |
| VASMPLISSTCDMVSAAYASTK | amino acids 29-50 of |
| | SEQ ID NO.: 7 |
| VSGAQEMVSSAK | amino acids 129-140 of |
| | SEQ ID NO.: 7 |

### EXAMPLE 5

GST-Tip47/3-(3,5-Ditritium-4-azidophenyl)-5-(3-chloro-thiophen-2-yl)-[1,2,4]-oxadiazole Binding Protocol

Full-length Tip47 cDNA was cloned into the pGEX-4T-1, a glutathione S-transferase (GST) gene fusion system (Amersham, Piscataway, N.J.) using standard methods. Briefly, PCR primers to the 5′ and 3′ region of the gene were designed to contain restriction sites that allowed for the in frame cloning of Tip47 into the pGEX-4T-1 vector. Subsequent to sequence verification, the pGEX-Tip47 construct was transformed into the E.Coli BL-21 strain. Tip47 was then expressed and purified by growing the E.Coli cells containing the pGEX-Tip47 according to the manufacturers suggested protocol.

In order to perform binding studies on Tip47, GST-Tip47 was immobilized on Sepharose. To begin, 10 μg of anti-GST antibody (cat. # sc-459, rabbit polyclonal, Santa Cruz Biotechnology, Santa Cruz, Calif.) was incubated with 20 μl of protein A Sepharose (Zymed, South San Francisco, Calif.), in TBS (pH 8.0), total volume 200 μl, for 1 hour at room temperature. Beads were washed 3 times with TBS (pH 8.0). 10 μg of GST-Tip47 (stock was kept as a 2 mg/ml solution in TBS pH 8.0 plus 2 mM DTT) was diluted to 200 μl TBS (pH 8.0) and added to the Protein A anti-GST Sepharose and incubated with rotation for 1 hour at room temperature. Beads were then washed 4 times with TBS (pH 8.0). To concentrate 3-(3,5-ditritium-4-azidophenyl)-5-(3-chloro-thiophen2-yl)-[1,2,4]-oxadiazole (Example 3), the compound was dried on a speed-vac and dissolved in DMSO at 1 mM. Compound was diluted to 2 μM in TBS (pH 8.0) and added to the beads. Final DMSO concentration was adjusted to 1%. Compound was incubated with beads for 1 hour at room temperature with rotation. Beads were washed 4 times with TBS (pH 8.0) and eluted with 100 μl of 100 mM Glycine-HCl buffer (pH 2.5) for 10 minutes at room temperature. Eluates were added to 5 ml of scintillation cocktail and counted using ³H protocol. Purified recombinant GST protein was used in place of GST-Tip47 to determine non-specific/background binding.

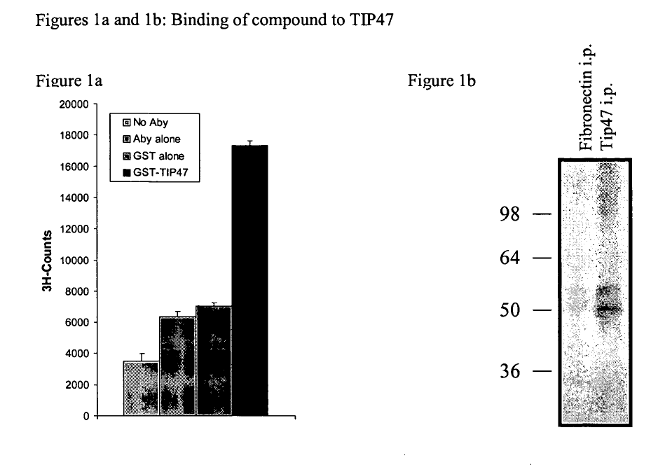

FIG. 1A shows 3-(3,5-ditritium-4-azidophenyl)-5-(3-chloro-thiophen-2-yl)-[1,2,4]-oxadiazole (Example 3) binding to GST-Tip47 immobilized on α-GST-Protein A-Sepharose.

### EXAMPLE 6

### Immunoprecipitation and Immunoblotting

For immunoprecipitations, T47D cells were first washed in PBS and then resuspended in CLB Buffer (10 mM HEPES, 10 mM NaCl, 1 mM KH₂PO₄, 5 mM NaHCO₃, 1 mM CaCl₂, 0.5 mM MgCl₂, 5 mM EDTA) plus 0.1% protease inhibitor cocktail (Sigma, St. Louis, Mo.). Cells were allowed to swell in 5 minutes at room temperature and then were homogenized in a tight fitting Dounce homogenizer with 50 strokes. Lysate was spun 2,200×g, 5 minutes, at 4° C. The supernatant was then spun at 100,000×g, 40 minutes at 4° C. This resulting supernatant was called T47D cytosol. Protein concentration determined by the D/C Protein Assay (Bio-Rad, Hercules, Calif.).

20 riM 3-(3,5-ditritium-4-azidophenyl)-5-(3-chloro-thiophen-2-yl)-[1,2,4]-oxadiazole (Example 3) (stock is 20 μM, 1 mCi/ml, 50 Ci/mmol) was added to 1 mg T47D cytosol in 1 ml CLB Buffer and incubated, rocking, for 30 minutes at room temperature. Lysates were then exposed to a Short Wave UV Source (254 nm) for 10 minutes.

Labeled lysates were pre-cleared with 50 μl solution of Protein A Sepharose (Zymed, South San Francisco, Calif.) for 2 hours at 4° C. 10 μg of either chicken anti-fibronectin IgY (Genway, San Diego, Calif.) or chicken anti-Tip47 IgY (Genway) were incubated with the lysates for 2 hours at 4° C. Then, 25 μg rabbit anti-chicken IgG was added to the lysates and incubated for 2 hours at 4° C. To bring down the complex, 50 μl Protein A Sepharose was incubated with the lysate and rocked over night at 4° C. This sepharose was then washed 6 times in CLB Buffer and resuspended in 2× sample buffer (Invitrogen Corporation) plus 40 mM DTT. Samples were subject to SDS-PAGE (Tris-Glycine gels, Invitrogen Corporation). The gel was stained with 1% Coomassie Brilliant Blue in 40% methanol, 7.5% acetic acid overnight at room temperature. Gels were destained in several changes of destainer (40% methanol, 7.5% acetic acid), incubated in Amplify (Amersham, Piscataway, N.J.) for 30 minutes at room temperature and then dried down at 80° C. for 2 hours on a gel dryer. Dried gels were put on Hyperfilm (Amersham) in a film cassette and placed at −80° C. Film was developed 4-7 days later.

FIG. 1B shows 3-(3,5-ditritium-4-azidophenyl)-5-(3-chloro-thiophen-2-yl)-[1,2,4]-oxadiazole (Example 3) binding to immunoprecipitated Tip47 from cell lysates.

For immunoblotting, cells were lysed in RIPA buffer (Upstate Biotechnologies, Lake Placid, N.Y.) and protein concentration was determined by the D/C Protein Assay (Bio-Rad, Hercules, Calif.). 35 μg protein was subject to SDS-PAGE (TrisGlycine gels, Invitrogen Corporation, Carlsbad, Calif.). Proteins were then transferred onto a PVDF membrane (Invitrogen Corporation) and blocked in 5% milk (Bio-Rad) and 1% BSA (Sigma, St. Louis, Mo.). Primary antibodies used include goat anti-actin (Santa Cruz Biotechnology, Santa Cruz, Calif.), mouse anti-p21 and mouse anti-cyclin D1 (BD Biosciences Pharmingen, San Diego, Calif.), and chicken anti-Tip47 (Genway, San Diego, Calif.), all used at 1 ug/ml in blocking buffer. Secondary antibodies used include bovine anti-goat (Santa Cruz Biotechnology), goat anti-mouse (Bio-Rad), and goat anti-chicken (Genway). Proteins were visualized with Super Signal West-Pico Luminol Enhancer Solution (Pierce, Rockford, Ill.).

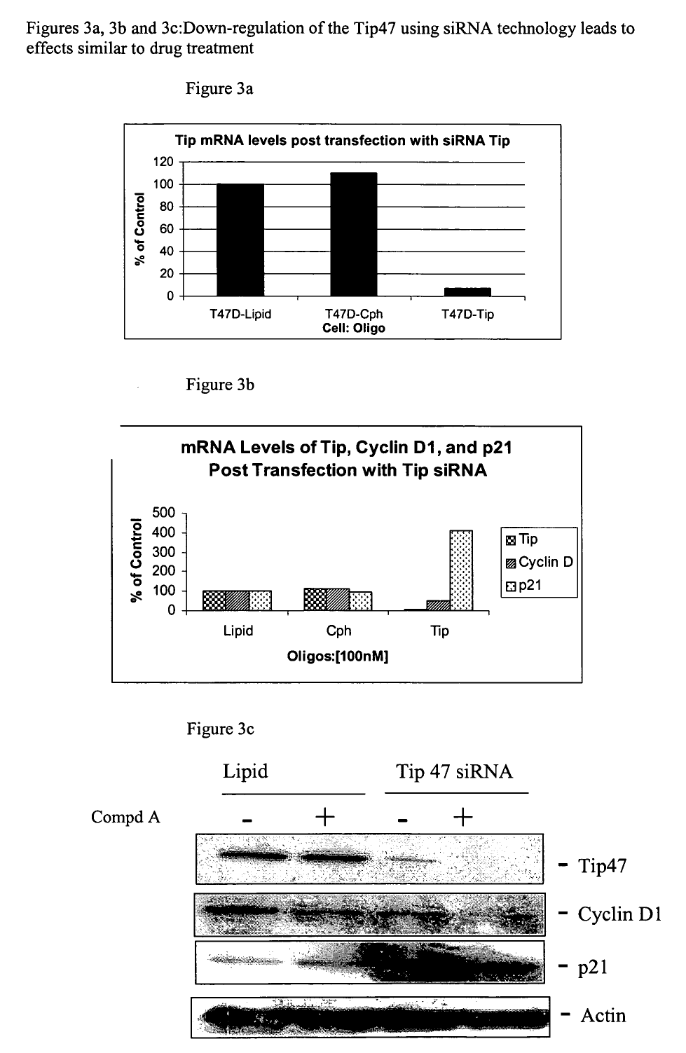

FIG. 3C shows the western blot data representing the down-regulation of Tip47 in siRNA transfected cells and its effect on genes of interest in the presence of compound and indicates the validation of the target.

### Example 7

### siRNA Transfections, cDNA Synthesis and Real-time PCR

Human TIP47 oligos were chemically synthesized by Ambion (Austin, Tex.). The target sequence for TIP47 siRNA was 5′ AACAGAGCTACTTCGTACGTC 3′ (nucleotides 695-716 of SEQ ID NO. 13). The control siRNA oligos and human cyclophilin were also from Ambion. T47D cells were grown to 50% confluence and allowed to attach overnight. siRNAs were transfected into the cells using Lipofectamine 2000 (Invitrogen Corporation, Carlsbad, Calif.) according to the manufacturer's instructions. The lipid complexes were added onto the cells and allowed to incubate for 48 h. The cells were then harvested for RNA and protein analysis.

For cDNA synthesis and quantitative PCR, total RNA was extracted using the TRIzol reagent (Invitrogen Corporation, Carlsbad, Calif.) according to the manufacturer's instructions. Total RNA was quantitated, denatured, and electrophoresed in an agarose-formaidehyde gei to determine integrity of total RNA. 2 μg of total RNA was then used to make cDNA by reverse transcription using the Retroscript cDNA synthesis kit (Ambion Austin, Tex.) according to the manufacturer's instructions. Quantitative PCR was done by Sybrgreen incorporation using the Quantitect kit (Qiagen, Valencia, Calif.) on the LightCycler (Roche Molecular Biochemicals, Mannheim, Germany) using standard conditions. Data was normalized against the housekeeping gene, cyclophilin. The cells transfected with cyclophilin as a control was normalized against glyceraldehyde phosphate dehydrogenase (GAPD).

FIG. 2 shows the gene expression profile of T47D cells in the presence of 5-(3-chlorothiophen-2-yl)-3-(5-chloro-pyridin-2-yl)-[1,2,4]-oxadiazole, showing the down regulation of cyclin D1.

FIG. 3A is the Realtime PCR data showing the down-regulation of the Tip47 at the mRNA level upon siRNA knock-down and validates TIP47 as the drug target.

FIG. 3B showing the down-regulation of the Tip47 and cyclin D1 at the mRNA level upon siRNA knock-down and validates TIP47 as the drug target.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A method of treating, preventing or ameliorating a disease responsive to induction of the caspase cascade in an animal, comprising administering to said animal a compound which binds specifically to a Tail Interacting Protein Related Apoptosis Inducing Protein (TIPRAIP), wherein said compound induces activation of the caspase cascade in said animal and said disease is treated, prevented or ameliorated; with the proviso that said compound is not 3-(4-azidophenyl)-5-(3-chloro-thiophen-2-yl)-[1,2,4]-oxadiazole or a substituted 3-aryl-5-aryl-[1,2,4]-oxadiazole.

2. The method of claim 1, wherein said disease is a hyperproliferative disease.

3. The method of claim 2, wherein said disease is cancer.

4. The method of claim 3, wherein said cancer is Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, or prostatic carcinomas.

5. The method of claim 1, wherein said disease is an inflammatory disease.

6. The method of claim 1, wherein said compound is identified by determining whether said compound binds specifically to TIPRAIP.

7. The method of claim 1, wherein said TIPRAIP is a tail interacting protein.

8. The method of claim 1, wherein said compound induces apoptosis in the cells of said animal within 24 to 48 hours, thereby treating, preventing or ameliorating said disease.

9. The method of claim 1, wherein the molecular weight of said compound is between 250 to 10,000 Daltons.

10. A method of identifying potentially therapeutic anticancer compounds comprising:
(a) contacting a Tail Interacting Protein Related Apoptosis Inducing Protein (TIPRAIP) with one or more test compounds; and
(b) monitoring whether said one or more test compounds binds to said TIPRAIP;
wherein compounds which bind said TIPRAIP are potentially therapeutic anticancer compounds.

11. The method of claim 10, wherein said TIPRAIP is a tail interacting protein.

12. The method of claim 10, wherein said determining whether said compound binds specifically to TIPRAIP comprises a competitive or noncompetitive homogeneous assay.

13. The method of claim 12, wherein said homogeneous assay is a fluorescence polarization assay or a radioassay.

14. The method of claim 10, wherein said determining whether said compound binds specifically to TIPRAIP comprises a competitive heterogeneous assay.

15. The method of claim 14, wherein said heterogeneous assay is a fluorescence assay or a radioassay.

16. The method of claim 10, wherein said TIPRAIP comprises a detectable label.

17. The method of claim 16, wherein said detectable label is selected from the group consisting of a fluorescent label and a radiolabel.

18. The method of claim 10, wherein the 3-(4-azidophenyl)-5-(3-chloro-thiophen-2-yl)-[1,2,4]-oxadiazole or substituted 3-aryl-5-aryl-[1,2,4]-oxadiazole comprises a detectable label.

19. The method of claim 18, wherein said detectable label is selected from the group consisting of a fluorescent label and a radiolabel.

20. The method of claim 10, wherein said TIPRAIP is present in cells in vitro.

21. A method of identifying potentially therapeutic anticancer compounds comprising:
(a) contacting said compound with an antibody to 3-(4-azidophenyl)-5-(3-chloro-thiophen-2-yl)-[1,2,4]-oxadiazole or a substituted 3-aryl-5-aryl-[1,2,4]-oxadiazole; and
(b) determining whether said compound binds to said antibody;
wherein compounds which bind said antibody are potentially therapeutic anticancer compounds.

22. A method of prognosing the efficacy of an anti-cancer TIPRAIP binding composition in a cancer patient comprising:
(a) taking a fluid or tissue sample from an individual manifesting a cancer;
(b) quantifying the total mRNA encoding TIPRAIP;
(c) calculating a ratio comprising the quantity of said mRNA to the average quantity of said mRNA in a fluid or tissue not manifesting said cancer;
wherein a ratio greater than 1 indicates that said anti-cancer TIPRAIP binding composition is efficacious.

23. A method of prognosing the efficacy of an anti-cancer TIPRAIP binding composition in a cancer patient comprising:
(a) taking a fluid or tissue sample from an individual manifesting a cancer;
(b) quantifying the TIPRAIP present in said sample;
(c) calculating a ratio comprising the quantity of said TIPRAIP to the average quantity of said TIPRAIP in a fluid or tissue not manifesting said cancer;
wherein a ratio greater than 1 indicates that said anti-canrcer TIPRAIP binding composition is efficacious.

24. A complex, comprising:
i) an TIPRAIP; and
ii) an TIPRAIP binding compound;
with the proviso that said TIPRAIP binding compound is not 3-(4-azidophenyl)-5-(3-chloro-thiophen-2-yl)-[1,2,4]-oxadiazole or a substituted 3-aryl-5-aryl-[1,2,4]-oxadiazole.

25. A detectably labeled 3-(4-azidophenyl)-5-(3-chloro-thiophen-2-yl)-[1,2,4]-oxadiazole or substituted 3-aryl-5-aryl-[1,2,4]-oxadiazole comprising i) 3-(4-azidophenyl)-5-(3-chloro-thiophen-2-yl)-[1,2,4]-oxadiazole or a substituted 3-aryl-5-aryl-[1,2,4]-oxadiazole;
ii) optionally a linker; and
iii) a label;
wherein said 3-(4-azidophenyl)-5-(3-chloro-thiophen-2-yl)-[1,2,4]-oxadiazole or substituted 3-aryl-5-aryl-[1,2,4]-oxadiazole is covalently linked to said label optionally via said linker.

26. The composition of claim 25, wherein said detectable label is biotin, a fluorescent label, or a radiolabel.

27. A composition comprising
i) 3-(4-azidophenyl)-5 -(3-chloro-thiophen-2-yl)-[1,2,4]-oxadiazole or a substituted 3-aryl-5-aryl-[1,2,4]-oxadiazole;
ii) optionally a linker; and
iii) a solid phase;
wherein said 3-(4-azidophenyl)-5-(3-chloro-thiophen-2-yl)-[1,2,4]-oxadiazole or substituted 3-aryl-5-aryl-[1,2,4]-oxadiazole is covalently linked to said solid phase optionally via said linker.

28. The composition of claim 27, wherein said solid phase is agarose or N-hydroxysuccinimidylcarboxyl-agarose.

29. A method of treating, preventing or ameliorating a disease responsive to induction of the caspase cascade in an animal, comprising administering to said animal a compound which
i) increases the level of cellular mRNA encoding transforming growth factor beta, cyclin-dependent kinase inhibitor 1A, insulin-like growth factor 2 receptor, or insulin-like growth factor binding protein 3; or
ii) decreases the level of cellular mRNA encoding cyclin D1; with the proviso that said compound is not 3-(4-azidophenyl)-5-(3-chloro-thiophen-2-yl)-[1,2,4]-oxadiazole or a substituted 3-aryl-5-aryl-[1,2,4]-oxadiazole.

30. A method of identifying potentially therapeutic anticancer compounds comprising:
(a) contacting cells with one or more test compounds; and
(b) monitoring
i) cellular increases in mRNA encoding transforming growth factor beta, cyclin-dependent kinase inhibitor 1A, insulin-like growth factor 2 receptor, or insulin-like growth factor binding protein 3; or
ii) cellular decreases in mRNA encoding cyclin D1;
wherein test compounds that cause said increases or decreases are potentially therapeutic anticancer compounds; with the proviso that said compounds do not include 3-(4-azidophenyl)-5-(3-chloro-thiophen-2-yl)-[1,2,4]-oxadiazole or a substituted 3-aryl-5-aryl-[1,2,4]-oxadiazole.

31. A method of treating, preventing or ameliorating a disease responsive to induction of the caspase cascade in an animal, comprising administering to said animal a compound which interferes with or prevents the binding of TIP-47 to insulin-like growth factor 2 receptor; with the proviso that said compound is not 3-(4-azidophenyl)-5-(3-chloro-thiophen-2-yl)-[1,2,4]-oxadiazole or a substituted 3-aryl-5-aryl-[1,2,4]-oxadiazole.

32. A method of identifying potentially therapeutic anticancer compounds comprising monitoring whether one or more test compounds interfere with or prevent the binding of TIP-47 to insulin-like growth factor 2 receptor; wherein test compounds that interfere or prevent said binding are potentially therapeutic anticancer compounds; with the proviso that said compounds do not include 3-(4-azidophenyl)-5-(3-chloro-thiophen-2-yl)-[1,2,4]-oxadiazole or a substituted 3-aryl-5-aryl-[1,2,4]-oxadiazole.
